(19) United States
(12) Patent Application Publication
(10) Pub. No.: US 2010/0011462 A1
(43) Pub. Date: Jan. 14, 2010

(54) FLAVIN MONOOXYGENASES AND TRANSCRIPTION FACTORS INVOLVED IN GLUCOSINOLATE BIOSYNTHESIS

(75) Inventors: Daniel James Kliebenstein, Davis, CA (US); Barbara Halkier, Frederiksberg (DK); Bjarne Gram Hansen, Frederiksberg (DK); Ida Elken Soenderby, Frederiksberg (DK)

Correspondence Address: MICHAEL BEST & FRIEDRICH LLP, ONE SOUTH PINCKNEY STREET, P O BOX 1806, MADISON, WI, 53701, US

(73) Assignee: University of Copenhagen

(21) Appl. No.: 12/438,332

(22) PCT Filed: Aug. 17, 2007
(86) PCT No.: PCT/IB07/02588

§ 371 Date: Feb. 20, 2009

Related U.S. Application Data
(60) Provisional application No. 60/839,116, filed on Aug. 22, 2006.

(51) Int. Cl. C12N 15/82; C12N 15/11; C12N 15/00; C12N 5/04; A01H 5/00; C12N 9/02; C12P 19/64; C07H 21/02

(52) U.S. Cl. 800/278; 536/23.2; 435/320.1; 435/468; 435/419; 800/298; 435/189; 435/73; 536/24.5

(57) ABSTRACT

The invention provides methods and materials relating generally to plant derived flavin-containing monooxygenases (FMOs) capable of catalysing oxidation of a thio- to a sulphinyl-group during glucosinolate biosynthesis. It further relates to plant derived MYB factors capable of transcriptional regulation of biosynthetic genes. These have utility in the modification of glucosinolate biosynthesis.

### TECHNICAL FIELD

The present invention relates generally to polypeptides such as transcription factors and oxygenase enzymes, and nucleic acids encoding them, which have utility e.g. in the modification of glucosinolate biosynthesis and modification.

### BACKGROUND ART

### Biosynthesis of GSLs

Glucosinolates (GSLs) are thioglycosides which occur in the Capparales (Rodman et al. (1996) Systematic Botany 21, 289-307). The molecule consists of a common glycone moiety and a variable aglycone side chain derived from an amino acid. In the majority of Capparalean families, GSLs have aromatic side chains derived from phenylalanine and branched side chains, derived from valine and leucine. However, the predominant GSLs in the Brassicaceae possess side chains derived from chain elongated forms of methionine and phenylalanine. Lower amounts of GSLs with indolyl side chains derived from tryptophan also occur. The methionine derived (‘aliphatic’) GSLs exhibit considerable variation in the length and structure of the side chain.

The biosynthesis of aliphatic GSLs can be considered in three parts:
Firstly, the initial entry of methionine into GSL biosynthesis and the development of chain elongation homologues of methionine.
Secondly the synthesis of the glycone moiety (i.e. the ‘GSL skeleton’)
Thirdly side chain modifications.

FIGS. 1a) and b) show some of the reactions catalysed in the second and third parts, including some of the enzymes and factors involved (see also Kliebenstein et al. (2001) The Plant Cell 13: 681-693).

### Hydrolysis of GSLs

FIG. 1c) shows some of the products resulting from GSL hydrolysis, including some of the enzymes and factors involved. The factor ‘ESP’, which favours epithionitrile formation, is discussed by Zhang et al., (2006) The Plant Cell 18: 1524-1536 and Matusheski et al. (2006) J Agric Food Chem 54: 2069-2076.

GSLs and their Economic and Biological Importance

Aliphatic GSLs in cruciferous crops are of economic and biological importance, largely as a result of hydrolytic products released upon tissue disruption. GSLs and their breakdown products are often collectively referred to as ‘mustard oils’.

For example, isothiocyanates derived from methylsulfinylalkyl GSLs via the activity of the enzyme myrosinase are associated with protection from carcinogens (Zhang et al. (1992). Proc. Natl. Acad. Sci. USA 89, 2399-2403). In particular, 4-methylsulphinylbutyl isothiocyanate (sulphoraphane), derived from the corresponding GSL 4-methylsulphinylbutyl glucosinolate, has previously been found to be a potent inducer of “phase 2” detoxifying enzymes, which has a role in detoxification of compounds (Zhang et al. (1992) The Plant Cell 18: 1524-1536). The corresponding heptyl- and octyl-GSLs have also been found to hold cancer preventive properties (Rose et al (2000). Carcinogenesis 21, 1983-1988).

Furthermore, sulphoraphane has been found to have an effect in bacteria that courses ulcers and stomach cancer (Fahey et al. (2002) PNAS 99, 7610-7615).

Moreover, many aliphatic GSLs have been implicated in mediating plant-herbivore interactions (Giamoustaris A & Mithen, R. F. (1995) Ann Appl Biol. 126, 347-363).

Additionally, GSLs and plants containing them have a role in biofumigation, wherein (for example) hydrolysis of glucosinolates in Brassica green manure or rotation crops leads to the release of biocidal compounds into the soil and the suppression of soil-borne pests and pathogens (J. A. Kirkegaard and M. Sawar, Plant and Soil, 201, 71-89, 1998).

By contrast to the above utilities, the presence of 2-hydroxy-3-butenyl and 2-hydroxy-4-pentenyl GSL in the seeds of Brassica oilseed crops, severely limits the use of rapeseed meal as a high protein animal feed as these two GSLs produce goitrogenic compounds upon ingestion, which cause goitre-like symptoms when fed to non-ruminating animals (poultry and pigs).

In view of the importance of GSL hydrolysis products it can be seen that the characterisation of activities involved in the GSL biosynthetic or metabolic pathways would provide a contribution to the art.

### SUMMARY OF THE INVENTION

The present inventors have identified genes in Arabidopsis coding for polypeptides affecting GSL biosynthesis.

### FMOs

One group of polypeptides of the present invention are enzymes which catalyze the conversion of methylthioalkyl GSLs (and desulfo-GSLs) to the corresponding methylsulfinylalkyl GSLs.

More specifically two genes have been identified in Arabidopsis which have been shown experimentally to catalyse oxygenation of (amongst others) methylthiobutyl glucosinolate to 4-methylsulphinylbutyl glucosinolate, i.e. the final step in the biosynthesis of 4-methylsulphinylbutyl glucosinolate, the precursor for sulphoraphane.

In addition, in planta data based on a knockout Arabidopsis mutant confirms the function of At1g65860, as the mutant has a reduced ratio of 4-methylsulphinylalkyl GSL to 4-methylthioalkyl GSL. Overexpression data has also been obtained wherein 4-methylthiobutyl glucosinolate levels are reduced when either At1g65860 or At1g62560 are expressed constitutively.

The genes are within the region of chromosome 1 containing the GS-OX locus described by Kliebenstein et al. (2001) Plant Physiol 126: 811-825. That publication discusses the genetic control of natural variation in Arabidopsis GSL accumulation. The putative GS-OX locus was mapped to chromosome 1 to a large region between AthGeneA and nga692 markers, although it was not further characterised.

These GS-OX enzymes have been characterised as flavin-containing monooxygenases (FMOs). Non-plant flavin-containing monooxygenases able to catalyse oxygenation of thiol groups have previously been identified (Ziegler, D. M, Drug Metabolism Reviews, 19, 33-62, 1988). Additionally Zhao et al. (2001) Science 291: 306-309 discusses a role for enzymes, which are said to be flavin monooxygenase-like enzymes, in auxin biosynthesis. The enzymes are said to catalyse the oxidation of an amino group of tryptamine to form N-hydroxyl tryptamine.

No plant-derived FMOs catalysing oxidation of a thio- to a sulphinyl-group have previously been characterised.

The FMO genes provide a powerful molecular mechanism for inter alia increasing the levels GSLs such as 4-methylsulphinylbutyl glucosinolate in plants (especially those with a high level of 4-methylthiobutyl glucosinolate). Another utility is in producing GSLs such as 4-methylsulphinylbutyl glucosinolate in fermentation tanks. These and other aspects are discussed in more detail below.

### MYBs

The present inventors have further identified three regulators of aliphatic GSLs in A. thaliana.

Over-expression of the individual MYB genes showed that they all had the capacity to increase the production of aliphatic glucosinolates in leaves and seeds and induce gene expression of aliphatic biosynthetic genes within leaves. In particular, overexpression of these regulators driven by the 35S promoter in Arabidopsis results in up to 2-fold increase in GSL flux. This yield may be increased by using 35S enhancer combined with endogenous promoters. In addition to affecting total content of aliphatic glucosinolates, the MYB genes altered the composition of the aliphatic glucosinolates present in the leaves.

Although a transcription factor has previously been implicated in the regulation of indole GSLs (Celenza et al. (2005) Plant Physiol 137: 253-262), regulators of biosynthetic genes in aliphatic GSLs have not been identified before. The identification of regulators specific for the biosynthesis of aliphatic GSLs allow metabolic engineering of these natural products to move from empirical to predictive engineering.

### DETAILED DESCRIPTION OF THE INVENTION

The overexpression or down-regulation of the genes of the invention described herein may be used to modulate in plants the levels of cancer preventive GSLs, improve flavour, enhance seed quality (e.g. by reducing goitrogenic compounds) as well as improve herbivore and pathogen resistance or biofumigative potential.

The characterisation of the genes provides methods for producing lines having these qualities by selective breeding or genetic manipulation.

These and other aspects of the invention are described in more detail below.

In the following aspects, nucleic acid according to the present invention may include cDNA, RNA, genomic DNA and modified nucleic acids or nucleic acid analogs (e.g. peptide nucleic acid). Where a DNA sequence is specified, e.g. with reference to a figure, unless context requires otherwise the RNA equivalent, with U substituted for T where it occurs, is encompassed. Nucleic acid molecules according to the present invention may be provided isolated and/or purified from their natural environment, in substantially pure or homogeneous form, or free or substantially free of other nucleic acids of the species of origin, and double or single stranded. Where used herein, the term “isolated” encompasses all of these possibilities. The nucleic acid molecules may be wholly or partially synthetic. In particular they may be recombinant in that nucleic acid sequences which are not found together in nature (do not run contiguously) have been ligated or otherwise combined artificially. Nucleic acids may comprise, consist, or consist essentially of, any of the sequences discussed hereinafter.

Aspects of the invention further embrace isolated nucleic acid comprising a sequence which is complementary to any of those discussed hereinafter.

### FMOs of the Invention

Thus according to one aspect of the present invention there is provided an isolated nucleic acid molecule which encodes an FMO capable of catalysing oxidation of a thio- to a sulphinyl-group. Such genes have not previously been identified in plants. This activity can be assayed as described herein e.g. by heterologous expression in E. coli with an appropriate thio-substrate, and in particular a thioalkyl GSL substrate, followed by HPLC analysis of products.

Preferably the isolated nucleic acid molecules are obtainable from a plant.

As described below, two FMOs from A. thaliana (encoded by At1g62560 and At1g65860) have been characterised by the inventors as catalyzing this reaction.

Additionally the inventors have established that phylogenetically these genes, with close homologues, are part of a cluster that is likely to be GSL specific. In particular At1g62570 and At1g62540 are part of a sub-cluster with At1g62560 and At1g65860, and are therefore believed to also catalyse the production of sulphinylalkyl GSLs.

The deduced amino acid sequences of these accessions (FMO polypeptides) are set out as SEQ ID NOs: 2, 4, 6, 8, and 10. Thus in one aspect of the invention, there is disclosed a nucleic acid encoding any of these polypeptides. The cDNA sequences of these accessions are set out as SEQ ID NOs: 1, 3, 5, 7 and 9. Other nucleic acids of the invention include those which are degeneratively equivalent to these.

The phylogenetic tree is shown in FIG. 2. In terms of the relationship between the encoded proteins, the minimal identity is 72%. When a further gene in the sub-cluster is included (At1g12140) the minimal identity is 68%. Thus a preferred mutual identity within the group of FMOs of the present invention is at least 68%, more preferably at least 72%. The level of similarity is even higher at 85% and 80% respectively. Thus a preferred mutual similarity within the group is at least 80%, more preferably at least 85%. Variants of the FMO sequences of the invention are discussed in more detail hereinafter.

Preferably the nucleic acid molecule encodes an FMO capable of catalysing oxidation of a thio- to a sulphinyl-group such as to form a sulphinylalkyl GSL. The inventors have also shown that FMOs of the present invention can oxidise desulfo-methylthioalkyl-GSLs, and it will be understood that where oxidation in respect of GSLs is discussed herein, the disclosure applies mutatis mutandis to desulfo-methylthioalkyl-GSLs also.

More preferably, the FMO is capable of catalysing oxidation of a thio- to a sulphinyl-group such as to form a methylsulphinylalkyl GSL, more preferably an omega-methylsulphinylalkyl GSL. By “omega” is meant the terminal carbon of the alkyl moiety e.g. C-4 in methylsulphinylbutyl GSL.

More preferably, the FMO is capable of catalysing oxidation of a thio- to a sulphinyl-group such as to form a methylsulphinylalkyl GSL, wherein the alkyl is selected from the group consisting of propyl, butyl, hexyl, pentyl, heptyl, or octyl. Such GSLs are present in different levels in many plants. Indeed, in crucifers, aliphatic GSLs are found with side chains up to n=11 (Daxenbichler et al (1991) Phytochemistry 30: 2623-2638). Thus it is proposed that other homologues identified and discussed herein may have different specificities for different chain lengths. In examples below it can be seen that At1g65860, At1g62570, At1g62560 and At1g62540 have a broad specificity towards all methylthioglucosinolates, whereas At1g12140 favours long-chain (especially octyl)methylthioalkyl GSLs. The inventors have demonstrated conversion levels of up to 80% have been achieved for preferred substrates and enzymes, as measured in the assays used herein.

### MYBs of the Invention

According to one aspect of the present invention there is provided an isolated nucleic acid molecule which encodes a transcriptional regulator of a biosynthetic gene encoding a polypeptide with aliphatic GSL-biosynthetic activity. As used herein, unless context demands otherwise, “biosynthetic gene” is used generally to mean any gene encoding a polypeptide in the biosynthetic pathway, including those involved in GSL intermediate or GSL product transport, inasmuch as these may affect production of GSLs.

“Transcriptional regulator” is a term well understood by those skilled in the art to mean a polypeptide or protein that binds to regulatory regions of a gene and controls (increases or reduces) gene expression. The regulators of the present invention have been shown to increase GSL-biosynthetic flux.

Such transcriptional regulators of aliphatic GSL-biosynthetic or transport activity have not previously been identified. This activity can be assayed as described herein e.g. by expression of the regulator in planta, followed by HPLC analysis and quantification of products.

Preferably the isolated nucleic acid molecules are obtainable from a plant. As described below, three highly related transcription factors of MYB-type from A. thaliana (encoded by At5g61420, At5g07690, and At5g07700) have been characterised by the inventors as having these properties.

Additionally the inventors have established that phylogenetically these genes, with close homologues, are part of a cluster that is likely to be GSL specific.

The deduced amino acid sequences of these accessions (MYB polypeptides) are set out as SEQ ID NOs: 12, 14, and 16. Thus in one aspect of the invention, there is disclosed a nucleic acid encoding any of these polypeptides. The CDS sequences of these accessions are set out as SEQ ID NOs: 11, 13, and 15. Other nucleic acids of the invention include those which are degeneratively equivalent to these.

A phylogenetic tree including the MYBs is shown in FIG. 6. In terms of the relationship between the encoded proteins, the minimal identity is 57%. Thus a preferred mutual identity within the group of MYBs of the present invention is at least 57%. The level of similarity is even higher at 69%. Thus a preferred mutual similarity within the group is at least 69%. Variants of the MYB sequences of the invention are discussed in more detail hereinafter.

### GSL Genes and Polypeptides of the Invention

For brevity, collectively the sequences encoding the 5 FMO and 3 MYB polypeptides discussed above may be described herein as “GSL genes of the invention” or the like. Likewise the encoded polypeptides are termed “GSL polypeptides of the invention”. It will be appreciated that where this term is used generally, it also applies to either of these two groups individually, and each of these sequences individually.

In each case the preferred FMO-encoding sequences are SEQ ID Nos 1, 3, 5 and 7 and the most preferred FMO-encoding sequences are SEQ ID Nos 1 and 3. The preferred FMO polypeptides are SEQ ID Nos 2, 4, 6, and 8 and the most preferred are SEQ ID Nos 2 and 4.

In each case the preferred MYB-encoding sequences are SEQ ID Nos 11, 13, and 15. The preferred MYB polypeptides are SEQ ID Nos 12, 14, and 16.

### Homologues and Other Variants of the Invention

In a further aspect of the present invention there are disclosed nucleic acids which are variants of the GSL genes of the invention discussed above.

A variant nucleic acid molecule shares homology with, or is identical to, all or part of the GSL genes or polypeptides of the invention discussed above.

They further share the relevant biological activity of the GSL genes of the invention.

For example, variants of the FMO polypeptides share the biological activity of being capable of catalysing oxidation of a thio- to a sulphinyl-group such as to form a methylsulphinylalkyl GSL, more preferably where the alkyl is selected from the group consisting of propyl, butyl, hexyl, pentyl, heptyl, or octyl.

Variants of the MYB polypeptides share the biological activity of transcriptionally regulating a biosynthetic gene encoding a polypeptide with (1) aliphatic GSL-biosynthetic activity; (2) GSL transport activity; (3) activity in the transport of intermediates in GSL biosynthesis.

Such variants may be used to alter the GSL content of a plant, as assessed by the methods disclosed herein. For instance a variant nucleic acids may include a sequence encoding a functional polypeptide (e.g. which may be a variant of any of SEQ ID Nos 2, 4, 6, 8, 10, 12, 14 or 16 above and which may cross-react with an antibody raised to said polypeptide). Alternatively they may include a sequence which interferes with the expression or activity of such a polypeptide (e.g. sense or anti-sense suppression of a GSL-gene of the invention).

Variants may also be used to isolate or amplify nucleic acids which have these properties.

Generally speaking variants may be:

(i) Novel, naturally occurring, nucleic acids, isolatable using the sequences of the present invention. They may include alleles (which will include polymorphisms or mutations at one or more bases) or pseudoalleles (which may occur at closely linked loci to the GSL genes of the invention). Also included are paralogues, isogenes, or other homologous genes belonging to the same families as the GSL genes of the invention. Also included are orthologues or homologues from other plant species.

Thus, included within the scope of the present invention are nucleic acid molecules which encode amino acid sequences which are homologues of GSL genes of the invention of Arabidopsis thaliana. Homology may be at the nucleotide sequence and/or amino acid sequence level, as discussed below. A homologue from a species other than Arabidopsis thaliana encodes a product which causes a phenotype similar to that caused by the Arabidopsis thaliana GSL genes of the invention. In addition, mutants, derivatives or alleles of these genes may have altered, e.g. increased or decreased, enzymatic activity or substrate specificity compared with wild-type.

(ii) Artificial nucleic acids, which can be prepared by the skilled person in the light of the present disclosure. Such derivatives may be prepared, for instance, by site directed or random mutagenesis, or by direct synthesis. Preferably the variant nucleic acid is generated either directly or indirectly (e.g. via one or more amplification or replication steps) from an original nucleic acid having all or part of the sequence of a GSL gene of the invention.

Also included are nucleic acids corresponding to those above, but which have been extended at the 3′ or 5′ terminus.

The term ‘variant’ nucleic acid as used herein encompasses all of these possibilities. When used in the context of polypeptides or proteins it indicates the encoded expression product of the variant nucleic acid.

Some of the aspects of the present invention relating to variants will now be discussed in more detail.

Homology (similarity or identity) may be assessed as set out in the Examples.

Homology may be at the nucleotide sequence and/or encoded amino acid sequence level. Preferably, the nucleic acid and/or amino acid sequence shares at least about 55%, 56%, 57%, 58%, 59%, 60%, 65%, or 70%, or 80% identity, most preferably at least about 90%, 95%, 96%, 97%, 98% or 99% identity.

Homology may be over the full-length of the relevant sequence shown herein, or may be over a part of it, preferably over a contiguous sequence of about or greater than about 20, 25, 30, 33, 40, 50, 67, 133, 167, 200, 233, 267, 300, 400 or more amino acids or codons, compared with a GSL polypeptide or gene of the invention as described above.

Thus a variant polypeptide encoded by a nucleic acid of the present invention may include within a GSL polypeptide sequence of the invention a single amino acid or 2, 3, 4, 5, 6, 7, 8, or 9 changes, about 10, 15, 20, 30, 40 or 50 changes, or greater than about 50, 60, 70, 80 or 90 changes.

In a further aspect of the invention there is disclosed a method of producing a derivative nucleic acid comprising the step of modifying any of the GSL genes of the present invention disclosed above.

Changes may be desirable for a number of reasons. For instance they may introduce or remove restriction endonuclease sites or alter codon usage.

Alternatively changes to a sequence may produce a derivative by way of one or more (e.g. several) of addition, insertion, deletion or substitution of one or more nucleotides in the nucleic acid, leading to the addition, insertion, deletion or substitution of one or more (e.g. several) amino acids in the encoded polypeptide.

Such changes may modify sites which are required for post translation modification such as cleavage sites in the encoded polypeptide; motifs in the encoded polypeptide for phosphorylation etc. Leader or other targeting sequences (e.g. membrane or golgi locating sequences) may be added to the expressed protein to determine its location following expression if it is desired to isolate it from a microbial system.

Other desirable mutations may be random or site directed mutagenesis in order to alter the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation. Also included are variants having non-conservative substitutions. As is well known to those skilled in the art, substitutions to regions of a peptide which are not critical in determining its conformation may not greatly affect its activity because they do not greatly alter the peptide's three dimensional structure. In regions which are critical in determining the peptides conformation or activity such changes may confer advantageous properties on the polypeptide. Indeed, changes such as those described above may confer slightly advantageous properties on the peptide e.g. altered stability or specificity.

Nucleic acid fragments may have utility in probing for, or amplifying, the sequence provided or closely related ones. Suitable lengths of fragment, and conditions, for such processes are discussed in more detail below.

The fragments may encode particular functional parts of the polypeptide (i.e. encoding a biological activity of it). Thus the present invention provides for the production and use of fragments of the full-length GSL polypeptides of the invention disclosed herein, especially active portions thereof. An “active portion” of a polypeptide means a peptide which is less than said full length polypeptide, but which retains its essential biological activity.

A “fragment” of a polypeptide means a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids. Fragments of the polypeptides may include one or more epitopes useful for raising antibodies to a portion of any of the amino acid sequences disclosed herein. Preferred epitopes are those to which antibodies are able to bind specifically, which may be taken to be binding a polypeptide or fragment thereof of the invention with an affinity which is at least about 1000× that of other polypeptides.

Particular regions, or domains, of GSL genes or polypeptides of the invention may have utility in their own right as follows.

An active portion of an FMO-polypeptide of the present invention retains the ability to catalyse oxidation of a methylthioalkyl GSL (or desulfo-methylthioalkyl-GSL) to the corresponding methylsulfinylalkyl GSL.

Individual MYB-polypeptide domains may be used to direct gene expression in a precise manner, for instance by the recognition of specific DNA sequences that represent elements in the promoters of their normal target genes. By creating fusion proteins, comprising the DNA binding domain (or domains) of MYB-polypeptides, and a heterologous activation or repression domain borrowed from another protein, the expression of target genes could be controlled. This may lead to a precise control of the expression of those genes that are normally targets of the MYB-polypeptides. Given that such genes are involved in GSL biosynthesis, their directed expression in other conditions may provide a useful means to control this. Furthermore, the use of fusions based on the DNA binding domains in conventional SELEX or one-hybrid experiments may be used to reveal the target genes or DNA sequences normally bound by the MYB-polypeptides. Thus nucleic acids encoding these domains, or fusion proteins comprising them, form one embodiment of this aspect of the present invention.

The provision of sequence information for the GSL genes of the invention of Arabidopsis thaliana enables the obtention of homologous sequences from other plant species. In particular, homologues may be easily isolated from Brassica spp (e.g. Brassica nigra, Brassica napus, Brassica oleraceae, Brassica rapa, Brassica carinata, Brassica juncea) as well as even remotely related cruciferous species. GSLs are also found in the genus Drypetes.

Thus a further aspect of the present invention provides a method of identifying and cloning FMO- or MYB-encoding homologues (i.e. genes which encode GSL-biosynthesis modifying polypeptides) from plant species other than Arabidopsis thaliana which method employs a GSL gene of the present invention. As discussed above, sequences derived from these may themselves be used in identifying and in cloning other sequences. The nucleotide sequence information provided herein, or any part thereof, may be used in a data-base search to find homologous sequences, expression products of which can be tested for ability to influence a plant characteristic. Alternatively, nucleic acid libraries may be screened using techniques well known to those skilled in the art and homologous sequences thereby identified then tested.

The present invention also extends to nucleic acid encoding an FMO-encoding homologue obtained using all or part of a nucleotide sequence shown as SEQ ID NOs 1, 3, 5 or 7 (or the corresponding genomic sequences of the relevant accessions).

The present invention also extends to nucleic acid encoding an MYB-encoding homologue obtained using all or part of a nucleotide sequence shown as SEQ ID NOs 11, 13, or 15 (or the corresponding genomic sequences of the relevant accessions).

These products will share a biological activity with a polypeptide of the invention, for example the ability to catalyse oxidation of a methylthioalkyl GSL (or desulfo-methylthioalkyl-GSL) to the corresponding methylsulfinylalkyl GSL (FMO variants) or to transcriptionally regulate a biosynthetic gene encoding a polypeptide with aliphatic GSL-biosynthetic or transport activities as discussed above (MYB variants).

In another embodiment the nucleotide sequence information provided herein may be used to design probes and primers for probing or amplification. An oligonucleotide for use in probing or PCR may be about 30 or fewer nucleotides in length (e.g. 18, 21 or 24). Generally specific primers are upwards of 14 nucleotides in length. For optimum specificity and cost effectiveness, primers of 16-24 nucleotides in length may be preferred. Those skilled in the art are well versed in the design of primers for use in processes such as PCR. If required, probing can be done with entire restriction fragments of the gene disclosed herein which may be 100's or even 1000's of nucleotides in length. Small variations may be introduced into the sequence to produce ‘consensus’ or ‘degenerate’ primers if required.

Such probes and primers form one aspect of the present invention.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the single stranded DNA fragments on the filter and binding determined. DNA for probing may be prepared from RNA preparations from cells. Probing may optionally be done by means of so-called ‘nucleic acid chips’ (see Marshall & Hodgson (1998) Nature Biotechnology 16: 27-31, for a review).

In one embodiment, a variant encoding a GSL-biosynthesis modifying polypeptide in accordance with the present invention is obtainable by means of a method which includes:

(a) providing a preparation of nucleic acid, e.g. from plant cells. Test nucleic acid may be provided from a cell as genomic DNA, cDNA or RNA, or a mixture of any of these, preferably as a library in a suitable vector. If genomic DNA is used the probe may be used to identify untranscribed regions of the gene (e.g. promoters etc.), such as are described hereinafter,
(b) providing a nucleic acid molecule which is a probe or primer as discussed above,
(c) contacting nucleic acid in said preparation with said nucleic acid molecule under conditions for hybridisation of said nucleic acid molecule to any said gene or homologue in said preparation, and,
(d) identifying said gene or homologue if present by its hybridisation with said nucleic acid molecule. Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include amplification using PCR (see below), RN'ase cleavage and allele specific oligonucleotide probing. The identification of successful hybridisation is followed by isolation of the nucleic acid which has hybridised, which may involve one or more steps of PCR or amplification of a vector in a suitable host.

Preliminary experiments may be performed by hybridising under low stringency conditions. For probing, preferred conditions are those which are stringent enough for there to be a simple pattern with a small number of hybridisations identified as positive which can be investigated further.

For example, hybridizations may be performed, according to the method of Sambrook et al. (below) using a hybridization solution comprising: 5×SSC (wherein ‘SSC’=0.15 M sodium chloride; 0.15 M sodium citrate; pH 7), 5×Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989):

Tₘ=81.5° C.+16.6 Log [Na+]+0.41(% G+C)−0.63(% formamide)−600/#bp in duplex

As an illustration of the above formula, using [Na+]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

It is well known in the art to increase stringency of hybridisation gradually until only a few positive clones remain. Other suitable conditions include, e.g. for detection of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

Thus this aspect of the present invention includes a nucleic acid including or consisting essentially of a nucleotide sequence of complementary to a nucleotide sequence hybridisable with any encoding sequence provided herein. Another way of looking at this would be for nucleic acid according to this aspect to be hybridisable with a nucleotide sequence complementary to any encoding sequence provided herein.

In a further embodiment, hybridisation of nucleic acid molecule to a variant may be determined or identified indirectly, e.g. using a nucleic acid amplification reaction, particularly the polymerase chain reaction (PCR). PCR requires the use of two primers to specifically amplify target nucleic acid, so preferably two nucleic acid molecules with sequences characteristic of a GSL gene of the present invention are employed. Using RACE PCR, only one such primer may be needed (see “PCR protocols; A Guide to Methods and Applications”, Eds. Innis et al, Academic Press, New York, (1990)).

Thus a method involving use of PCR in obtaining nucleic acid according to the present invention may include:

(a) providing a preparation of plant nucleic acid, e.g. from a seed or other appropriate tissue or organ,
(b) providing a pair of nucleic acid molecule primers useful in (i.e. suitable for) PCR, at least one of said primers being a primer according to the present invention as discussed above,
(c) contacting nucleic acid in said preparation with said primers under conditions for performance of PCR,
(d) performing PCR and determining the presence or absence of an amplified PCR product. The presence of an amplified PCR product may indicate identification of a variant.

In all cases above, if need be, clones or fragments identified in the search can be extended. For instance if it is suspected that they are incomplete, the original DNA source (e.g. a clone library, mRNA preparation etc.) can be revisited to isolate missing portions e.g. using sequences, probes or primers based on that portion which has already been obtained to identify other clones containing overlapping sequence.

If a putative naturally occurring homologous sequence is identified, its role in GSL biosynthesis can be confirmed, for instance by methods analogous to those used in the Examples below, or by generating mutants of the gene (e.g. by screening the available insertional-mutant collections) and analyzing the GSL content of the plants. Alternatively the role can be inferred from mapping appropriate mutants to see if the homologue lies at or close to an appropriate locus.

In a further embodiment, antibodies raised to a GSL polypeptide or peptide of the invention can be used in the identification and/or isolation of variant polypeptides, and then their encoding genes. Thus, the present invention provides a method of identifying or isolating a GSL-biosynthesis modifying polypeptide, comprising screening candidate polypeptides with a polypeptide comprising the antigen-binding domain of an antibody (for example whole antibody or a fragment thereof) which is able to bind a GSL polypeptide of the invention, or preferably has binding specificity for such a polypeptide. Methods of obtaining antibodies are described hereinafter.

Candidate polypeptides for screening may for instance be the products of an expression library created using nucleic acid derived from a plant of interest, or may be the product of a purification process from a natural source. A polypeptide found to bind the antibody may be isolated and then may be subject to amino acid sequencing. Any suitable technique may be used to sequence the polypeptide either wholly or partially (for instance a fragment of the polypeptide may be sequenced). Amino acid sequence information may be used in obtaining nucleic acid encoding the polypeptide, for instance by designing one or more oligonucleotides (e.g. a degenerate pool of oligonucleotides) for use as probes or primers in hybridization to candidate nucleic acid.

### Uses of GSL-Biosynthesis Modifying Nucleic Acids

As used hereinafter, unless the context demands otherwise, the term “GSL-biosynthesis modifying nucleic acid” is intended to cover any of the GSL-genes of the present invention and variants thereof described above, particularly those variants encoding polypeptides sharing the biological activity of a GSL-polypeptide of the invention, for example the ability to catalyse oxidation of a methylthioalkyl GSL (or desulfo-methylthioalkyl-GSL) to the corresponding methylsulfinylalkyl GSL (FMO variants) or to transcriptionally regulate a biosynthetic gene encoding a polypeptide with aliphatic GSL-biosynthetic or transport activity as discussed above (MYB variants).

The term “GSL-biosynthesis modifying polypeptide” should be interpreted accordingly.

The present invention provides for inter alia reduction or increase in GSL quality or quantity in plants. This allows for production of better seed quality (e.g. in Brassica napus), increase of cancer preventive GSL's in cruciferous salads such as e.g. Eruca sativa, and enhancement of herbivore and pathogen resistance in cruciferous crop plants.

As noted above, important dietary GSLs such as 4-methylsulphinylbutyl glucosinolate are only found in fairly low levels in many vegetables, including Brassica vegetables and other cruciferous salads (McNaughton et al. 2003, British Journal Of Nutrition 90 (3): 687-697). It is therefore desirable to get plant with a higher content. Such plants can be used either directly in human consumption or they will be a good source for extraction of 4-methylsulphinylbutyl glucosinolate. Thus, for example, GSL-biosynthesis modifying nucleic acids may be transformed into plants such as Brassica vegetables and other cruciferous salads to increase the level of sulphoraphane present when the plants are consumed.

In different embodiments, the present invention provides means for manipulation of total levels of GSLs in plants such as oilseeds and horticultural crucifers through modification of GSL biosynthesis, e.g. by up or down regulating GSL-biosynthesis modifying nucleic acids.

In one aspect of the present invention, the GSL-biosynthesis modifying nucleic acid described above is in the form of a recombinant and preferably replicable vector.

“Vector” is defined to include, inter alia, any plasmid, cosmid, phage or Agrobacterium binary vector in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication).

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al., 1989, Cold Spring Harbor Laboratory Press or Current Protocols in Molecular Biology, Second Edition, Ausubel et al. eds., John Wiley & Sons, 1992.

Specifically included are shuttle vectors by which is meant a DNA vehicle capable, naturally or by design, of replication in two different host organisms, which may be selected from actinomycetes and related species, bacteria and eucaryotic (e.g. higher plant, yeast or fungal cells).

A vector including nucleic acid according to the present invention need not include a promoter or other regulatory sequence, particularly if the vector is to be used to introduce the nucleic acid into cells for recombination into the genome.

Preferably the nucleic acid in the vector is under the control of, and operably linked to, an appropriate promoter or other regulatory elements for transcription in a host cell such as a microbial, e.g. bacterial, or plant cell. The vector may be a bifunctional expression vector which functions in multiple hosts. In the case of genomic DNA, this may contain its own promoter or other regulatory elements (optionally in combination with a heterologous enhancer, such as the 35S enhancer discussed in the Examples below). The advantage of using a native promoter is that this may avoid pleiotropic responses. In the case of cDNA this may be under the control of an appropriate promoter or other regulatory elements for expression in the host cell

By “promoter” is meant a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment, the promoter is an inducible promoter.

The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Thus nucleic acid according to the invention may be placed under the control of an externally inducible gene promoter to place expression under the control of the user. An advantage of introduction of a heterologous gene into a plant cell, particularly when the cell is comprised in a plant, is the ability to place expression of the gene under the control of a promoter of choice, in order to be able to influence gene expression, and therefore GSL biosynthesis, according to preference. Furthermore, mutants and derivatives of the wild-type gene, e.g. with higher or lower activity than wild-type, may be used in place of the endogenous gene.

Thus this aspect of the invention provides a gene construct, preferably a replicable vector, comprising a promoter (optionally inducible) operably linked to a nucleotide sequence provided by the present invention, such as the GSL-biosynthesis modifying gene.

Particularly of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy R R D ed) Oxford, BIOS Scientific Publishers, pp 121-148). Suitable vectors may include plant viral-derived vectors (see e.g. EP-A-194809).

Suitable promoters which operate in plants include the Cauliflower Mosaic Virus 35S (CaMV 35S). Other examples are disclosed at pg 120 of Lindsey & Jones (1989) “Plant Biotechnology in Agriculture” Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

If desired, selectable genetic markers may be included in the construct, such as those that confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate). Positive selection system such as mannose isorase (Haldrup et al. 1998 Plant molecular Biology 37, 287-296) to make constructs that do not rely on antibiotics.

The present invention also provides methods comprising introduction of such a construct into a plant cell or a microbial (e.g. bacterial, yeast or fungal) cell and/or induction of expression of a construct within a plant cell, by application of a suitable stimulus e.g. an effective exogenous inducer.

In a further aspect of the invention, there is disclosed a host cell containing a heterologous construct according to the present invention, especially a plant or a microbial cell.

The term “heterologous” is used broadly in this aspect to indicate that the gene/sequence of nucleotides in question (e.g. encoding a GSL-biosynthesis modifying polypeptide) have been introduced into said cells of the plant or an ancestor thereof, using genetic engineering, i.e. by human intervention. A heterologous gene may replace an endogenous equivalent gene, i.e. one which normally performs the same or a similar function, or the inserted sequence may be additional to the endogenous gene or other sequence. Nucleic acid heterologous to a plant cell may be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

The host cell (e.g. plant cell) is preferably transformed by the construct, which is to say that the construct becomes established within the cell, altering one or more of the cell's characteristics and hence phenotype e.g. with respect to GSL biosynthesis.

Nucleic acid can be introduced into plant cells using any suitable technology, such as a disarmed Ti-plasmid vector carried by Agrobacterium exploiting its natural gene transfer ability (EP-A-270355, EP-A-0116718, NAR 12 (22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11.

Agrobacterium transformation is widely used by those skilled in the art to transform dicotyledonous species.

Thus a further aspect of the present invention provides a method of transforming a plant cell involving introduction of a construct as described above into a plant cell and causing or allowing recombination between the vector and the plant cell genome to introduce a nucleic acid according to the present invention into the genome.

The invention further encompasses a host cell transformed with nucleic acid or a vector according to the present invention (e.g. comprising the GSL-biosynthesis modifying nucleotide sequence) especially a plant or a microbial cell. In the transgenic plant cell (i.e. transgenic for the nucleic acid in question) the transgene may be on an extra-genomic vector or incorporated, preferably stably, into the genome. There may be more than one heterologous nucleotide sequence per haploid genome.

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

Plants which include a plant cell according to the invention are also provided.

Preferred plant species in which it may be preferred to modify GSL biosynthesis according to the present invention are any in which such biosynthesis occurs naturally e.g. Brassicales and Drypetes.

Where the intention is to use FMO-genes of the present invention or variants thereof, it is preferred that the plant target naturally produces methylthioalkyl GSLs.

It is believed that almost all cruciferous crops have at least one genotype with some methylthioalkyl GSL content.

More preferably, the plant comprises a methyl-thio-alkyl-GSL, wherein the alkyl is selected from the group consisting of propyl, butyl, pentyl, hexyl heptyl, or octyl.

4-Methylsulfinylbutyl GSL and 3-methylsulfinylpropyl GSL GSLs are found in several cruciferous vegetables, but are most abundant in broccoli varieties (syn. calabrese: Brassica oleracea L. var. italica) which lack a functional allele at the GSL-ALK locus.

The most important crops for modification of meal quality are oilseed forms of Brassica spp. (e.g. B. napus, B. rapa (syn B. campestris), B. juncea, B. carinata).

For enhancement of flavour and cancer preventive properties the most important species are B. oleracea (including e.g. Broccoli and Cauliflower), horticultural forms of B. napus (e.g. swedes [=rutabaga, spp. napobrassica], oil seed rape) and B. rapa (including both turnips and chinese cabbage [=pakchois]), cruciferous salads (including e.g. Eruca sativa and Diplotaxis tenuifolia) and horticultural forms of Raphanus (e.g. Radish (Raphanus sativa)).

The plant background may preferably be one in which the breakdown of GSLs is directed (naturally, or by genetic manipulation) towards isothiocyanates to get e.g. sulforophane.

GSLs may also be modified in condiment mustard forms of Sinapis alba (white/yellow mustard), B. juncea (brown/Indian mustard) and B. nigra (black mustard). All of these species are targets for enhancement of pest and disease resistance via GSL modification. Modifications for enhanced disease and pest resistance includes modifications to leaf and root GSLs to enhance the biofumigation potential of crucifers when used as green manures and as break crops in cereal rotations.

The levels of GSLs in commercially grown broccoli are relatively low compared to those found in salad crops such as rocket (Eruca Sativa and Diplotaxis tenuifolia) which accumulates 4-methylthiobutyl glucosinolate (Nitz et al 2002, Journal Of Applied Botany-Angewandte Botanik 76 (3-4): 82-86; McNaughton et al. 2003, British Journal Of Nutrition 90 (3): 687-697). Rocket is one particular preferred target.

Plant backgrounds such as those above may be natural or transgenic e.g. for one or more other genes relating to GSL biosynthesis. For FMO or MYB encoding genes, specifically preferred backgrounds are: those that have a 4-carbon allele or null allele at that species' GS-Elong locus; those that have the null allele at that species' GS-AOP locus (since the presence of Alk or OHP at this locus decreases the concentration of sulfinyl GLS).

For plants in which it is desired to down-regulate FMO or MYB encoding genes (e.g. with antisense, amiRNA or hairpin silencing constructs—see below) the preferred backgrounds are those which have the GS-OH locus leading to pro-goitrin.

In addition to the regenerated plant, the present invention embraces all of the following: a clone of such a plant, seed, selfed or hybrid progeny and descendants (e.g. F1 and F2 descendants). The invention also provides a plant propagule from such plants, that is any part which may be used in reproduction or propagation, sexual or asexual, including cuttings, seed and so on. It also provides any part of these plants, which in all cases include the plant cell or heterologous GSL-biosynthesis modifying DNA described above.

A plant according to the present invention may be one which does not breed true in one or more properties. Plant varieties may be excluded, particularly registrable plant varieties according to Plant Breeders' Rights.

### Polypeptides and Expression Products

The present invention also encompasses the expression product of any of the coding GSL-biosynthesis modifying nucleic acid sequences disclosed and methods of making the expression product by expression from encoding nucleic acid therefore under suitable conditions, which may be in suitable host cells.

Use of a recombinant FMO polypeptide of the invention, or variant thereof, to convert methylthioalkyl GSL (or desulfo-methylthioalkyl-GSL) to the corresponding methylsulfinylalkyl GSL forms one aspect of the present invention.

As disclosed herein, At1g65860, At1g62570, At1g62560 and At1g62540 have been shown to have a broad specificity towards the tested methylthioalkyls GSLs whereas At1g12140 mainly converts long-chain (especially octyl)methylthioalkyls-into methylsulfinylalkyl GSLs.

Therefore where the GSL is shorter chain (e.g. less than octyl), At1g65860, At1g62570, At1g62560 or At1g62540 may be preferred.

Use of a recombinant MYB polypeptide of the invention, or variant thereof as a DNA-binding protein, or more specifically a modulator of transcription, or most preferably as a transcriptional regulator of a biosynthetic gene encoding a polypeptide with aliphatic GSL-biosynthetic or transport activity or GSL-intermediate transport activity, forms another aspect of the invention.

As shown in the Examples below while MYB28 affects the level of both long and short chain aliphatic glucosinolates (including methylsulfinyloctyl glucosinolate, 8MSO), it appears that that MYB29 and MYB76 mainly affect the level of shorter-chain aliphatic GSLs.

Therefore where the GSL is longer chain (e.g. octyl), use or manipulation of MYB28 may be preferred.

### Down-Regulation

In addition to use of the nucleic acids of the present invention for production of functional GSL-biosynthesis modifying polypeptides the information disclosed herein may also be used to reduce the activity of GSL-biosynthesis modifying activity in cells in which it is desired to do so.

This may be desirable, for instance, to prevent the accumulation of undesirable GSLs in plants (such as 2-hydroxy-3-butenyl glucosinolate (progoitrin) in rapeseed)—see FIG. 1.

Down-regulation of expression of a target gene may be achieved using anti-sense technology.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Rothstein et al, 1987; Smith et al, (1988) Nature 334, 724-726; Zhang et al, (1992) The Plant Cell 4, 1575-1588, English et al., (1996) The Plant Cell 8, 179-188. Antisense technology is also reviewed in Bourque, (1995), Plant Science 105, 125-149, and Flavell, (1994) PNAS USA 91, 3490-3496.

An alternative to anti-sense is to use a copy of all or part of the target gene inserted in sense, that is the same, orientation as the target gene, to achieve reduction in expression of the target gene by co-suppression. See, for example, van der Krol et al., (1990) The Plant Cell 2, 291-299; Napoli et al., (1990) The Plant Cell 2, 279-289; Zhang et al., (1992) The Plant Cell 4, 1575-1588, and U.S. Pat. No. 5,231,020. Further refinements of the gene silencing or co-suppression technology may be found in WO95/34668 (Biosource); Angell & Baulcombe (1997) The EMBO Journal 16, 12:3675-3684; and Voinnet & Baulcombe (1997) Nature 389: pg 553.

The complete sequence corresponding to the coding sequence (in reverse orientation for anti-sense) need not be used. For example fragments of sufficient length may be used. It is a routine matter for the person skilled in the art to screen fragments of various sizes and from various parts of the coding sequence to optimise the level of anti-sense inhibition. It may be advantageous to include the initiating methionine ATG codon, and perhaps one or more nucleotides upstream of the initiating codon. A further possibility is to target a conserved sequence of a gene, e.g. a sequence that is characteristic of one or more genes, such as a regulatory sequence.

The sequence employed may be about 500 nucleotides or less, possibly about 400 nucleotides, about 300 nucleotides, about 200 nucleotides, or about 100 nucleotides. It may be possible to use oligonucleotides of much shorter lengths, 14-23 nucleotides, although longer fragments, and generally even longer than about 500 nucleotides are preferable where possible, such as longer than about 600 nucleotides, than about 700 nucleotides, than about 800 nucleotides, than about 1000 nucleotides or more.

It may be preferable that there is complete sequence identity in the sequence used for down-regulation of expression of a target sequence, and the target sequence, although total complementarity or similarity of sequence is not essential. One or more nucleotides may differ in the sequence used from the target gene. Thus, a sequence employed in a down-regulation of gene expression in accordance with the present invention may be a wild-type sequence (e.g. gene) selected from those available, or a variant of such a sequence in the terms described above. The sequence need not include an open reading frame or specify an RNA that would be translatable.

Further options for down regulation of gene expression include the use of ribozymes, e.g. hammerhead ribozymes, which can catalyse the site-specific cleavage of RNA, such as mRNA (see e.g. Jaeger (1997) “The new world of ribozymes” Curr Opin Struct Biol 7:324-335, or Gibson & Shillitoe (1997) “Ribozymes: their functions and strategies form their use” Mol Biotechnol 7: 242-251).

Anti-sense or sense regulation may itself be regulated by employing an inducible promoter in an appropriate construct.

Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi) (See also Fire (1999) Trends Genet. 15: 358-363, Sharp (2001) Genes Dev. 15: 485-490, Hammond et al. (2001) Nature Rev. Genes 2:1110-1119 and Tuschl (2001) Chem. Biochem. 2: 239-245).

RNA interference is a two step process. First, dsRNA is cleaved within the cell to yield short interfering RNAs (siRNAs) of about 21-23 nt length with 5′ terminal phosphate and 3′ short overhangs (˜2 nt) The siRNAs target the corresponding mRNA sequence specifically for destruction (Zamore P. D. Nature Structural Biology, 8, 9, 746-750, (2001)

Thus in one embodiment, the invention provides double stranded RNA comprising a sequence encoding part of a GSL polypeptide of the present invention or variant (homologue) thereof, which may for example be a “long” double stranded RNA (which will be processed to siRNA, e.g., as described above). These RNA products may be synthesised in vitro, e.g., by conventional chemical synthesis methods.

RNAi may be also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)).

Thus siRNA duplexes containing between 20 and 25 bps, more preferably between 21 and 23 bps, of the GSL-genes of the present invention sequence form one aspect of the invention e.g. as produced synthetically, optionally in protected form to prevent degradation. Alternatively siRNA may be produced from a vector, in vitro (for recovery and use) or in vivo.

Accordingly, the vector may comprise a nucleic acid sequence encoding a GSL-gene of the present invention (including a nucleic acid sequence encoding a variant or fragment thereof), suitable for introducing an siRNA into the cell in any of the ways known in the art, for example, as described in any of references cited herein, which references are specifically incorporated herein by reference.

In one embodiment, the vector may comprise a nucleic acid sequence according to the invention in both the sense and antisense orientation, such that when expressed as RNA the sense and antisense sections will associate to form a double stranded RNA. This may for example be a long double stranded RNA (e.g., more than 23 nts) which may be processed in the cell to produce siRNAs (see for example Myers (2003) Nature Biotechnology 21:324-328).

Alternatively, the double stranded RNA may directly encode the sequences which form the siRNA duplex, as described above. In another embodiment, the sense and antisense sequences are provided on different vectors.

Another methodology known in the art for down-regulation of target sequences is the use of “microRNA” (miRNA) e.g. as described by Schwab et al 2006, Plant Cell 18, 1121-1133. This technology employs artificial miRNAs, which may be encoded by stem loop precursors incorporating suitable oligonucleotide sequences, which sequences can be generated using well defined rules in the light of the disclosure herein. Thus, for example, in one aspect there is provided a nucleic acid encoding a stem loop structure including a sequence portion of one of the target GSL-genes of the invention of around 20-25 nucleotides, optionally including one or more mismatches such as to generate miRNAs (see e.g. http://wmd.weigelworld.org/bin/mirnatools.pl). Such constructs may be used to generate transgenic plants using conventional techniques.

These vectors and RNA products may be useful for example to inhibit de novo production of the GSL polypeptides of the present invention in a cell. They may be used analogously to the expression vectors in the various embodiments of the invention discussed herein.

Thus the present invention further provides the use of any of the sequence above, for example: variant GSL-biosynthesis modifying nucleotide sequence, or its complement (e.g. in the context of any of the technologies discussed above); double stranded RNA with appropriate specificity as described above; a nucleic acid precursor of siRNA or miRNA as described above; for down-regulation of gene expression, particularly down-regulation of expression of the GSL-biosynthesis modifying gene or homologue thereof, preferably in order to modify GSL biosynthesis in a plant.

As shown in the Examples below, analysis of a double knockout in MYB28 and MYB29 identified an emergent property of the system since the very, very low level of aliphatic glucosinolates in these plants could not be predicted by the chemotype of the single knockouts. Thus the MYB regulatory genes disclosed herein appear to have evolved both overlapping and specific regulatory capacities, and appear to be the main regulators of aliphatic glucosinolates in Arabidopsis.

Thus double- or even triple-knockouts (or other down-regulated mutants) may be preferred in manipulating phenotypes, in the relevant aspects of the invention described herein.

### Combinations of GSL-Related Genes

The GSL-genes of the present invention and variants thereof may be used in combination with any other gene, such as transgenes involved in GSL biosynthesis or other phenotypic trait or desirable property.

By use of a combination of genes, plants or microorganisms (e.g. bacteria, yeasts or fungi) can be tailored to enhance production of desirable precursors, or reduce amounts of undesirable metabolism.

For example the use of MYB-encoding nucleic acids in conjunction with FMO-encoding nucleic acids may maximise GSL flux to desirable nutraceutical GSLs. Metabolic engineering in this way, with a combination of overexpression regulators of aliphatic GSLs and the final step in methylsulphinyl GSL, makes it realistic to engineer even very high levels of desirable GSLs.

The effect of the combination of MYB and FMO polypeptides parallels published reports of the use of anthocyanin regulators and reductases. Thus overexpression of anthocyanin regulators resulted in red tobacco plants due to very high accumulation of the anthocyanin color compounds, and overexpression of anthocyanin regulators combined with anthocyanin reductase resulted in accumulation of proanthocyanin (see e.g. Borevitz J O, Xia Y, Blount J, Dixon R A and Lamb C (2000) The Plant Cell, 12, 2383-2393. Activation tagging identifies a conserved MYB regulator of phenylpropanoid biosynthesis; Xie D, Sharma S B, Wright E, Wang Z-Y and Dixon R A (2006) The Plant Journal, 45, 895-907. Metabolic engineering of proanthocyanidns through co-expression of anthocyanidin reductase and the PAP1 MYB transcription factor).

Thus host cells and plants in which heterologous copies of MYB and FMO genes are both present form a particular preferred embodiment of the present invention.

Other genes which it may be desirable to manipulate or introduce in concert with FMO or MYB encoding genes include those of the GS-Elong locus; the GS-AOP locus or the GS-OH locus, which are discussed above.

### Methods of Altering Phenotype

Up- and down-regulation of the activity of GSL polypeptides of the present invention and variants thereof enables modifications to be made to meal quality of oilseeds crucifers, cancer preventive activity and flavour of horticultural crucifers, and/or resistance to herbivores and pathogens and biofumigative activity.

Methods of the invention may be used to produce non-naturally occurring GSLs, or GSLs which are non-naturally occurring in the species into which they are introduced—these products forming a further aspect of the present invention.

Methods used herein may be used, for example, to increase levels of methylsulfinylalkyl GSL for improved nutraceutical potential or increased methylthioalkyl GSL for improved flavour or increasing biofumigative activity or potential. The methods of the present invention may include the use of GSL-biosynthesis modifying nucleic acids of the invention, optionally in conjunction with the manipulation (e.g. over-expression or down-regulation) other genes affecting GSL biosynthesis known in the art.

The invention further provides a method of influencing or affecting GSL biosynthesis in a plant, the method including causing or allowing transcription of a heterologous GSL-biosynthesis modifying nucleic acid sequence as discussed above within the cells of the plant. The step may be preceded by the earlier step of introduction of the GSL-biosynthesis modifying nucleic acid into a cell of the plant or an ancestor thereof.

More specifically the FMO-encoding genes provided by the present invention may be used to modify biosynthesis of glucosinolates, preferably in respect of side chain modification.

For example the invention provides various methods of influencing a GSL biosynthetic catalytic activity in a cell (preferably a plant cell). The methods comprise the step of modifying in that cell the activity (e.g. nature or concentration) of an enzyme capable of catalysing oxidation of a methylthioalkyl GSL (or desulfo-methylthioalkyl-GSL) to the corresponding methylsulfinylalkyl GSL or a transcription factor capable of regulating a biosynthetic gene encoding a polypeptide with aliphatic GSL-biosynthetic or transport activity.

Such methods will usually form a part of, possibly one step in, a method of producing a GSL, or modifying the production of a GSL, in a plant. Preferably the method will employ a nucleic acid encoding an FMO polypeptide of the present invention, or variant thereof, as described above or a MYB polypeptide of the present invention, or variant thereof, as described above.

In a further aspect of the present invention there is disclosed a method of producing a GSL, or modifying the production of a GSL, said method comprising the step of using an enzyme which catalyses oxidation of a methylthioalkyl GSL (or desulfo-methylthioalkyl-GSL) to the corresponding methylsulfinylalkyl GSL or a transcription factor capable of regulating a biosynthetic gene encoding a polypeptide with aliphatic GSL-biosynthetic or transport activity.

The methods of the present invention embrace both the in vitro and in vivo production, or manipulation, of one or more GSLs.

For example, enzymes such as FMOs may be employed in fermentation tanks to convert methylthioalkyl GSLs (e.g. 4MTB, 7MTB, 8MTB) into the corresponding methylsulfinylalkyl GSLs via expression in microorganisms such as e.g. E. coli, yeast and filamentous fungi and so on. As noted above, FMOs may be used in these organisms in conjunction with other biosynthetic genes.

As an alternative to microorganisms, cell suspension cultures of GSL-producing, FMO-expressing plant species may be cultured in fermentation tanks. Overexpression of regulators of the metabolon (e.g. MYB factors) can activates the metabolon in this undifferentiated state (see for example Grotewold et al. (Engineering Secondary Metabolites in Maize Cells by Ectopic Expression of Transcription Factors, Plant Cell, 10, 721-740, 1998) which discloses the production of high amounts of deoxyflavonoids in undifferentiated maize cell suspension culture by overexpression of one or two transcription factors).

As discussed in more detail below, in this and other aspects of the invention, when used in vitro the enzyme will generally be in isolated, purified, or semi-purified form. Optionally it will be the product of expression of a recombinant nucleic acid molecule.

Likewise the in vivo methods will generally involve the step of causing or allowing the transcription of, and then translation from, a recombinant nucleic acid molecule encoding the enzyme.

In further aspects of the present invention there are disclosed:

A method of producing a GSL, or modifying the production of a GSL, said method comprising use of a nucleic acid molecule encoding an enzyme capable of catalysing oxidation of a methylthioalkyl GSL (or desulfo-methylthioalkyl-GSL) to the corresponding methylsulfinylalkyl GSL.

A method of producing a GSL, or modifying the production of a GSL, said method comprising use of an enzyme to catalyse oxidation of a methylthioalkyl GSL (or desulfo-methylthioalkyl-GSL) to the corresponding methylsulfinylalkyl GSL.

A method of producing a GSL, or modifying the production of a GSL, said method comprising use of a nucleic acid molecule encoding a transcription factor capable of regulating a biosynthetic gene encoding a polypeptide with aliphatic GSL-biosynthetic or transport activity.

A method of producing a GSL, or modifying the production of a GSL, said method comprising use of a transcription factor capable of regulating a biosynthetic gene encoding a polypeptide with aliphatic GSL-biosynthetic or transport activity.

A method of producing a GSL, or modifying the production of a GSL, said method comprising use of a plant, plant cell, or microorganism transformed with a nucleic acid molecule encoding an enzyme capable of catalysing oxidation of a methylthioalkyl GSL (or desulfo-methylthioalkyl-GSL) to the corresponding methylsulfinylalkyl GSL.

A method of producing a GSL, or modifying the production of a GSL, said method comprising use of a plant, plant cell, or microorganism expressing a heterologous enzyme to catalyse oxidation of a methylthioalkyl GSL (or desulfo-methylthioalkyl-GSL) to the corresponding methylsulfinylalkyl GSL.

As described in the introduction, GSL compounds play a role in seed quality, cancer preventive properties, herbivore and pathogen resistance, biofumigation activity and so on.

Thus the present invention includes a method of altering any one or more of these characteristics in a plant, comprising use of a method as described hereinbefore. Specific examples include alteration of flavour or nutritional (or ‘nutraceutical’) value of a plant or plant product.

As disclosed herein, At1g65860, At1g62570, At1g62560 and At1g62540 have been shown to have a broad specificity towards the tested methylthioalkyls GSLs whereas At1g12140 mainly converts long-chain (especially octyl)methylthioalkyls-into methylsulfinylalkyl GSLs.

Therefore in all of the aspects of the invention described herein comprising use of an enzyme (or nucleic acid encoding an enzyme) capable of catalysing oxidation of a methylthioalkyl GSL (or desulfo-methylthioalkyl-GSL) to the corresponding methylsulfinylalkyl GSL, where the GSL is shorter chain (e.g. less than octyl), an FMO enzyme encoded by At1g65860, At1g62570, At1g62560 or At1g62540 may be preferred.

As shown in the Examples below while MYB28 affects the level of both long and short chain aliphatic glucosinolates (including methylsulfinyloctyl glucosinolate, 8MSO), it appears that that MYB29 and MYB76 mainly affect the level of shorter-chain aliphatic GSLs.

Therefore in all of the aspects of the invention described herein related to use of transcription factors (or nucleic acids encoding such factors) to manipulate GSLs, where the GSL is longer chain (e.g. octyl), manipulation of the transcription factor MYB28 (or nucleic acid encoding the same) may be preferred.

### Marker Assisted Breeding

Much of the foregoing discussed has been concerned with the genetic modification of plants by use of artificial recombinant nucleic acids. However the disclosure of the GSL-genes of the present invention also provides novel methods of plant breeding and selection, for instance to manipulate phenotype such as meal quality of oilseeds crucifers, anticarcinogenic activity and flavour of horticultural crucifers, and/or resistance to herbivores and pathogens.

A further aspect of the present invention provides a method for assessing the GSL phenotype of a plant, the method comprising the step of determining the presence and/or identity of a GSL-biosynthesis modifying allele therein comprising the use of a nucleic acid as described above. Such a diagnostic test may be used with transgenic or wild-type plants, and such plants may or may not be mutant lines e.g. obtained by chemical mutagenesis.

The use of diagnostic tests for alleles allows the researcher or plant breeder to establish, with full confidence and independent from time consuming biochemical tests, whether or not a desired allele is present in the plant of interest (or a cell thereof), whether the plant is a representative of a collection of other genetically identical plants (e.g. an inbred variety or cultivar) or one individual in a sample of related (e.g. breeders' selection) or unrelated plants.

The present disclosure provides sufficient information for a person skilled in the art to obtain genomic DNA sequence for any given new or existing allele (e.g. the various homologues discussed above) and devise a suitable nucleic acid- and/or polypeptide-based diagnostic assay. DNA genomically linked to the alleles may also be sequenced for flanking markers associated with the allele. The sequencing polymorphisms that may be used as genetic markers may, for example, be single nucleotide polymorphisms, multiple nucleotide polymorphisms or sequence length polymorphisms. The polymorphisms could be detected directly from sequencing the homologous genomic sequence from the different parents or from indirect methods of indiscriminantely screening for visualizable differences such as CAPs markers or DNA HPLC.

In designing a nucleic acid assay account is taken of the distinctive variation in sequence that characterises the particular variant allele.

For example GSL genes of the invention or homologues thereof can be used in marker assisted selection programmes to reduce antinutritional GLS in seed meals of Brassica oilseed crops (e.g. e.g. B. napus, B. rapa (syn B. campestris), B. juncea, B. carinata), to enhance cancer preventive GSL in Brassica vegetables crop and other cruciferous salads and to modify plant-herbivore interactions.

For example, markers developed from the homologues for use in breeding increased levels of methylsulfinylalkyl GSL for improved nutraceutical potential or increased methylthioalkyl GSL for improved flavour. As noted above, breeding may also be used to alter disease resistance and biofumigation potential resulting in a better breaking crop e.g. in previously uncultivated or disease-infested land.

Thus in one embodiment of the present invention, a method is described which employs the use of DNA markers derived from or associated with GSL genes of the present invention (or homologues thereof from Brassicas and other cruciferous plants) that segregate with specific GSL profiles. In one embodiment of this method, the use of the DNA markers, or more specifically markers known as flanking QTLs (quantitative trait loci) are used to select the genetic combination in Brassicas that leads to elevated levels of methylsulfinylalkyl GSLs.

Thus aspects of the invention embrace the selective increase of cancer preventive GSL derivatives in cruciferous crop species, and to cruciferous crop species with enhanced levels of cancer preventive GSL derivatives and in particular edible Brassica vegetables and cruciferous salads with elevated levels of the cancer preventive GSL derivatives methylsulfinylalkyl isothiocyanate. The present invention also provides methods for selection of genetic combinations of broccoli containing high levels of cancer preventive GSL derivatives and methods to evaluate the cancer preventive properties of these genetic combinations.

In a breeding scheme based on selection and selfing of desirable individuals, nucleic acid or polypeptide diagnostics for the desirable allele or alleles in high throughput, low cost assays as provided by this invention, reliable selection for the preferred genotype can be made at early generations and on more material than would otherwise be possible. This gain in reliability of selection plus the time saving by being able to test material earlier and without costly phenotype screening is of considerable value in plant breeding.

Nucleic acid-based determination of the presence or absence of one or more desirable alleles may be combined with determination of the genotype of the flanking linked genomic DNA and other unlinked genomic DNA using established sets of markers such as RFLPs, microsatellites or SSRs, AFLPs, RAPDs etc. This enables the researcher or plant breeder to select for not only the presence of the desirable allele but also for individual plant or families of plants which have the most desirable combinations of linked and unlinked genetic background. Such recombinations of desirable material may occur only rarely within a given segregating breeding population or backcross progeny. Direct assay of the locus as afforded by the present invention allows the researcher to make a stepwise approach to fixing (making homozygous) the desired combination of flanking markers and alleles, by first identifying individuals fixed for one flanking marker and then identifying progeny fixed on the other side of the locus all the time knowing with confidence that the desirable allele is still present.

Accordingly in this embodiment of the present invention one potential method to produce a GSL-biosynthesising plant having elevated levels of methylsulfinylalkyl GSLs is described which comprises:

I.) Preparing F1 hybrid plants;
II.) Analyzing F1 hybrids by screening with DNA markers derived from or associated with GSL genes of the present invention (or homologues thereof), and selecting hybrids for backcrossing with one parental line;
III.) Analysis of DNA markers derived from or associated with GSL genes of the present invention (or homologues thereof) in individual plants of the B1 (Backcross 1) generation and selection of lines with the optimum GSL genotype as related to the DNA markers derived from or associated with GSL genes of the present invention;
IV.) One or two further rounds of DNA marker assisted backcrossing with selection of plants as per II to generate production quality germplasm).

This method is only an example and not all inclusive. DNA marker assisted selection utilizing DNA markers derived from or associated with GSL genes of the present invention (or homologues thereof) can be successfully utilized in any genetic crossing scheme to optimize the efficiency of obtaining the desired GSL phenotype.

GSLs from the plants of the plants or methods of the invention may be isolated and commercially exploited.

This product can be used as dietary supplement or in functional food e.g. in products analysis to “Brassica tea” which is said to contain around 15 mg sulphoraphane/tea bag (www.brassicatea.com).

### Antibodies

Purified protein according to the present invention, or a fragment, mutant, derivative or variant thereof, e.g. produced recombinantly by expression from encoding nucleic acid therefor, may be used to raise antibodies employing techniques which are standard in the art. Antibodies and polypeptides comprising antigen-binding fragments of antibodies may be used in identifying homologues from other species as discussed further below.

Methods of producing antibodies include immunising a mammal (e.g. human, mouse, rat, rabbit, horse, goat, sheep or monkey) with the protein or a fragment thereof. Antibodies may be obtained from immunised animals using any of a variety of techniques known in the art, and might be screened, preferably using binding of antibody to antigen of interest. For instance, Western blotting techniques or immunoprecipitation may be used (Armitage et al, 1992, Nature 357: 80-82). Antibodies may be polyclonal or monoclonal.

As an alternative or supplement to immunising a mammal, antibodies with appropriate binding specificity may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies raised to a polypeptide or peptide can be used in the identification and/or isolation of homologous polypeptides, and then the encoding genes. Thus, the present invention provides a method of identifying or isolating a polypeptide with the desired function (in accordance with embodiments disclosed herein), comprising screening candidate polypeptides with a polypeptide comprising the antigen-binding domain of an antibody (for example whole antibody or a suitable fragment thereof, e.g. scFv, Fab) which is able to bind a polypeptide or fragment, variant or derivative thereof according to the present invention or preferably has binding specificity for such a polypeptide. Specific binding members such as antibodies and polypeptides comprising antigen binding domains of antibodies that bind and are preferably specific for a polypeptide or mutant, variant or derivative thereof according to the invention represent further aspects of the present invention, particularly in isolated and/or purified form, as do their use and methods which employ them.

Candidate polypeptides for screening may for instance be the products of an expression library created using nucleic acid derived from an plant of interest, or may be the product of a purification process from a natural source. A polypeptide found to bind the antibody may be isolated and then may be subject to amino acid sequencing. Any suitable technique may be used to sequence the polypeptide either wholly or partially (for instance a fragment of the polypeptide may be sequenced). Amino acid sequence information may be used in obtaining nucleic acid encoding the polypeptide, for instance by designing one or more oligonucleotides (e.g. a degenerate pool of oligonucleotides) for use as probes or primers in hybridization to candidate nucleic acid, or by searching computer sequence databases, as discussed further below.

Antibodies may be modified in a number of ways. Indeed the term “antibody” should be construed as covering any specific binding substance having a binding domain with the required specificity. Thus, this term covers antibody fragments, derivatives, functional equivalents and homologues of antibodies, including any polypeptide comprising an immunoglobulin binding domain, whether natural or synthetic.

The invention will now be further described with reference to the following non-limiting Figures and Examples. Other embodiments of the invention will occur to those skilled in the art in the light of these.

Any title and sub-title in the description herein is for convenience only and should not be interpreted as limiting the disclosure in any way.

The disclosure of all references cited herein, inasmuch as it may be used by those skilled in the art to carry out the invention, is hereby specifically incorporated herein by cross-reference.

### FIGURES

FIG. 1a): shows the biosynthesis of the GS core structure in A. thaliana. The initial substrate is either a proteinogenic amino acid or a chain-elongated amino acid. Gluc, glucose (from Pietrowski et al. 2004 J Biol Chem 279: 50717-50725). FIG. 1b) shows the side Chain Modifications of Methionine-Derived Glucosinolates in Arabidopsis. Potential side chain modifications for the elongated methionine derivative C₄ dihomomethionine are shown. Steps with natural variation in Arabidopsis are shown in boldface to the right or left of each enzymatic arrow with the name of the corresponding QTL (from Kliebenstein et al. (2001) The Plant Cell 13: 681-693). FIG. 1c) shows a generic model of GSL hydrolysis. TFF is the thiocyanate-forming factor and ESP epithiospecifier protein (adapted from Matusheski et al. (2006) J Agric Food Chem 54: 2069-2076).

FIG. 2—Phylogenetic analysis of protein sequences for the complete genomic complement of all flavin-monooxygenases within Arabidopsis thaliana and Oryzae sativa. Neighbor joining with 1000 bootstrap permutations were used to evaluate the relationships of all FMO proteins. Putative chemical reactions are shown in gray boxes with the branches at which they first occurred. Blue sequences are from Oryzae and black are from Arabidopsis.

FIG. 3—Enzymatic activity of heterologously expressed At1g65860 in E. coli spheroplasts using the arabinose inducible pBad TOPO® TA Expression system (Invitrogen). 50 μg total E. coli protein were used for each assay and allowed to proceed for 1 hour at 30° C. 4-methylsulfinylbutyl glucosinolate and desulfo-methylsulfinylbutyl glucosinolate production were quantified by HPLC (monitored at 229 nm). Compound identities were confirmed by comparison of both retention times, UV light absorption profiles and mass by LC/MS with those of authentic standards.

(A) Enzyme assay with 4-methylthiobutyl glucosinolate as substrate. X-axis show concentration of substrate in assay.
(B) Enzyme assay with desulfo-4-methylthiobutyl glucosinolate as substrate.

The X-axis shows concentration of substrate in assay.

At1g65860: Assays with spheroplast from arabinose induced E. coli expressing His-tagged At1g65860

Empty; Assays with spheroplast from arabinose induced E. coli with empty pBad vector

FIG. 4—Ratios of sulphinyl/thio GSLs for each specific chain length in Arabidopsis thaliana ecotype Columbia-0 offspring from a heterozygous segregating knock out in At1g65860 (Salk line 079493). Glucosinolates were extracted from leaves from 24 day plants. The ratios are the mean of extractions from six individual plants ±one standard deviation. 3MSP: 3-methylsulfinylpropyl glucosinolate 3MTP: 3-methylthiopropyl glucosinolate; 4MSB: 4-methylsulfinylbutyl glucosinolate; 4MTB: 4-methylthiobutyl glucosinolate; 5MSP: 5-methylsulfinylpentyl glucosinolate; 5MTP: 5-methylthiopentyl glucosinolate; 6MSH: 6-methylsulfinylhexyl glucosinolate; 6MTH: 6-methylthiohexyl glucosinolate; 7MSH: 7-methylsulfinylheptyl glucosinolate, 7MTH: 7-methylthioheptyl glucosinolate; 8MSO: 8-methylsulfinyloctyl glucosinolate; 8MTO: 8-methylthiooctyl glucosinolate.

Homozygous KO: homozygous knock out in At1g65860 from segregating heterozygous knock out in At1g65860 (Salk line 079493).
Heterozygous KO: heterozygous knock out in At1g65860 from segregating heterozygous knock out in At1g65860 (Salk line 079493).
Salk WT: wild type in At1g65860 from segregating heterozygous knock out in At1g65860 (Salk line 079493).

FIG. 5—4-methylthiobutyl glucosinolate levels in rosette leaves from 24 day old wild type columbia and transgenic At1g65860 and At1g62560 overexpression lines. Quantities are given in nmol/mg fresh weight ±one standard deviation and are the mean of extractions from four individual plants. Two independent lines were analysed for each construct. The lines H and AT for 35S overexpression of At1g65860 and line 9 and 11 for 35S overexpression of At1g62560.

4MTB: 4-methylthiobutyl glucosinolate.
35S: cauliflower mosaic virus 35S promoter

FIG. 6—A clade in the Myb transcription factor family tree. This clade contains the three Myb transcription factors of interest, AtMyb28, AtMyb29 and AtMyb76, and their three closest related genes, AtMyb34 (ATR1), AtMyb51 and AtMyb122 of which ATR1 has been characterized as a regulator of indole GSLs (Celenza et al 2005) (Figure extract from Stracke et al. 2001).

FIG. 7—The overexpression constructs used in expression of the Myb transcription factors. The 35S promoter (35S prom) derived from the cauliflower mosaic virus 35S promoter drives strong constitutive expression of the coding sequence (CDS) of the gene of interest. Its 35S terminator (35S term) ensures the termination of transcription. The 35Senh-overexpressor consists of the enhancer element (35S enh) from the 35S promoter. This enhancer element enhances the expression of the gene's own natural promoter (prom) when this, along with the genomic locus (encompassing the transcribed region of the gene) is cloned behind the enhancer.

FIG. 8—HPLC chromatogram of desulfoGSL profiles of 35S:Myb76, line 6 (blue line) and wildtype Col-0 (black line). 20 μl sample was injected on the LC-MS and separated on a Zorbax SB-AQ RPC18 column (4.6 mm×250 mm, 5 um) kept at 25° C. at a flow rate of 1 ml/min. The GSLs were detected at 229 nm. Single desulfoGSLs were identified according to their ion-trace chromatograms and mass spectra ([M+Na]⁺ adduct ions). Full names of GSLs are given in the abbreviation list.

FIG. 9—Indole and aliphatic GSL levels in leaves of 22 days old Col0 Arabidopsis wildtypes and selected Arabidopsis lines overexpressing Myb28, Myb29 or Myb76. Overexpression was obtained by cloning the CDS of the genes behind the cauliflower mosaic virus 35S promoter (e.g. 35S:Myb28) or by cloning the promoter, along with the genomic locus (encompassing the transcribed region of the gene) behind the 35Senhancer (35Senh-Myb76) from the cauliflower mosaic virus. Error bars represent +/−standard deviation for n=5-6 and n=14 (wildtype). FW=fresh weight.

FIG. 10—Indole and aliphatic GSL levels in leaves of 24 days old Col0 Arabidopsis wildtypes and selected Arabidopsis lines overexpressing Myb28. Overexpression was obtained by cloning the promoter, along with the genomic locus (encompassing the transcribed region of the gene) behind the 35S enhancer from cauliflower mosaic virus. Error bars represent +/−standard deviation for n=4 and n=14 (wildtype). MP16A=empty vector control. FW=fresh weight.

FIG. 11—Heterologous expression of At1g62560 in E. coli using 4MTB as substrate, with an empty vector as control.

FIG. 12—4-6 fold increase in 4MSB levels in seeds in overexpressors of At1g62560 and At1g65860.

FIG. 13—Heterologous expression of At1g62540 in E. coli using 4MTB as substrate, with an empty vector as control.

FIG. 14—Glucosinolate profile of seeds in 35S: At1g62540 line.

FIG. 15—Heterologous expression of At1g12140 in E. coli using desulfo-glucosinolates from glucosinolates from Arabidopsis Col-0 seeds as substrate mix. This confirms At1g12140 has S-oxygenation activity, with high activity with 8MTO.

FIG. 16—Glucosinolate profile of seeds in 35S: At1g12140 line

FIG. 17—Heterologous expression of At1g62570 in E. coli using desulfo-glucosinolates from glucosinolates from Arabidopsis Col-0 seeds as substrate mix, with an empty vector as control.

FIG. 18—At1g62570 overexpression in seeds, compared to wild-type.

FIG. 19—Expression of Sulfur Utilization Biosynthetic Pathways in 35S:MYB lines. Nested ANOVAs were utilized on microarray data to test for altered expression of the major sulfur utilization biosynthetic pathways as described. The pathways linking one major metabolite to another with statistically significant altered expression are shown as colored arrows. Red shows that the 35S:MYB lines led to increased transcript levels for the biosynthetic pathway in comparison to wild-type, while blue shows decreased transcript levels. Dark color represents a change of 50 percent or more while the lighter color shows a change of less than 50 percent. MYB28 illustrates the comparison of transcript levels in 35S:MYB28 lines versus Col-0. MYB29 illustrates the comparison of transcript levels in 35S:MYB29 lines versus Col-0. MYB76 illustrates the comparison transcript levels in 35S:MYB76 plants versus Col-0.

FIG. 20—Altered Transcript Levels for Genes in the Biosynthetic Pathway of Aliphatic Glucosinolates in the 35S:MYB lines. Nested ANOVAs were utilized on microarray data to test for altered transcript levels for biosynthetic genes in the aliphatic glucosinolate pathway. Each arrow represents a specific biosynthetic process with the transcript alteration for each of the different enzymes indicated as separate rows of boxes. From left to right, the boxes in each row illustrates the comparison of the transcript levels in, respectively, the 35S:MYB28, 35S:MYB29 and 35S:MYB76 transgenes versus Col-0. Genes with a statistically significant altered transcript increase in the given 35S:MYB line are shown as red while those with a decrease are in blue. Dark color represents a change of 50 percent or more while the lighter color shows a change of less than 50 percent.

A. Altered transcript accumulation for the biosynthetic genes.
B. Altered transcript accumulation for the MYB transcription factors by the different 35S transgenes.
C. Altered 4-MSB accumulation by the different 35S transgenes.
D. Altered 8-MSO accumulation by the different 35S transgenes.
E. Altered total aliphatic glucosinolate accumulation by the different 35S transgenes.

FIG. 21—Overlap in altered gene regulation between the 35S:MYB over-expressor lines. Each ring of the Venn diagram shows the number of genes whose transcript level was statistically significantly altered by the given 35S:MYB transgene. Statistical significance was determined by individual gene ANOVAs using a FDR of 0.05. The bottom diagram shows the predicted number of genes in each intersection under the assumption that the MYB genes have independent regulatory functions.

FIG. 22—Characterization by RT-PCR of transcript levels in myb28-1, myb29-1, myb29-2, myb76-1, myb76-2 and myb28-1 myb29-1 mutants.

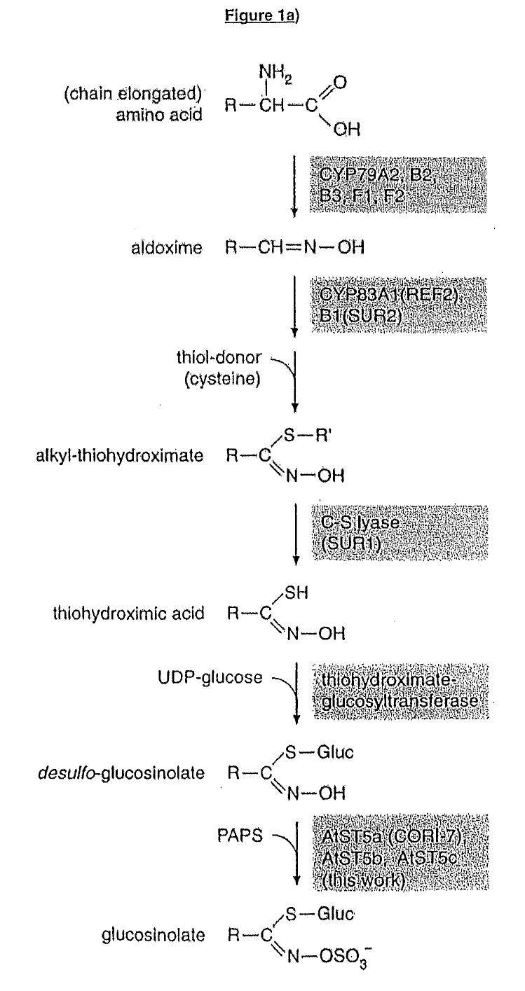

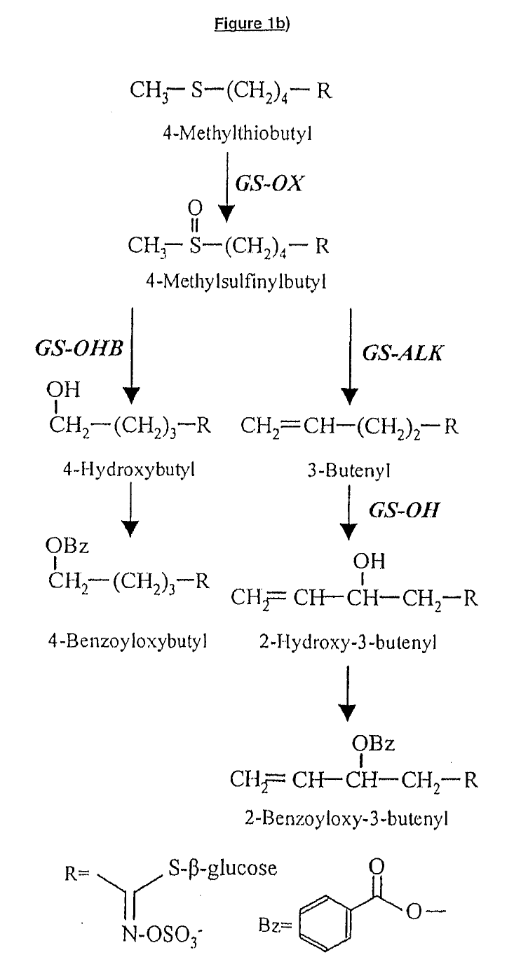

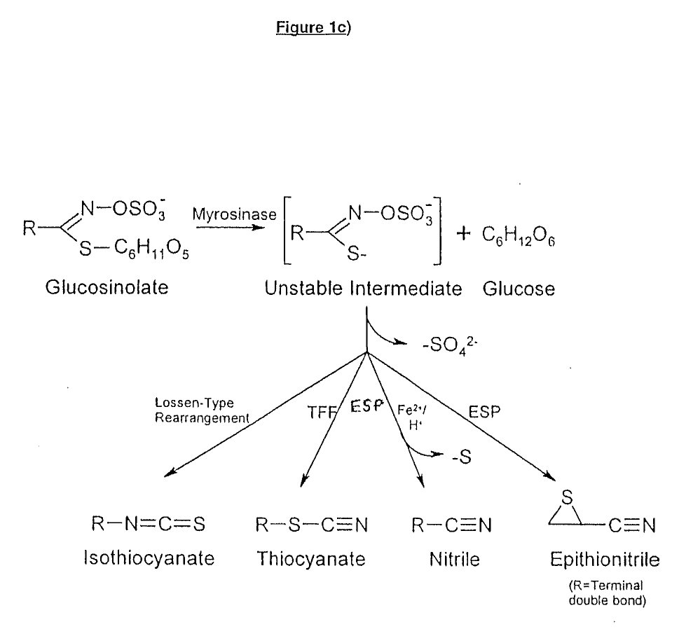

A. Diagram of the MYB28, MYB29 and MYB76 genes with exons given as black boxes and 5′UTR, 3′UTR and introns given as black lines. The T-DNA insertion site in myb28-1 is located in the 5′UTR, the T-DNA insertion site in myb29-1 and myb29-2 is located in the third exon and 5′UTR, respectively, and the T-DNA insertion of myb76-1 and myb76-2 is located in the first exon and first intron, respectively. Arrows marked F and R show the approximate positions of the primers used for RT-PCR.
B. Steady state foliar mRNA transcript levels of MYB28, MYB29 and MYB76 and various aliphatic biosynthetic genes in wild-type Col-0, myb28-1, myb29-1, myb29-2, myb76-1 and myb76-2 and myb28-1 myb29-1 mutants as measured by RT-PCR in 23-25 days old plants. Each mutant is displayed with its corresponding wild-type. A PCR for actin was used as a loading control. Amplification was shown to be in the logarithmic phase.
C. Steady state foliar mRNA transcript levels of MYB28, MYB29 and MYB76 and various aliphatic biosynthetic genes in wild-type Col-0 and the myb28-1 myb29-1 mutant as measured by RT-PCR in 25 days old plants. A PCR for actin was used as a loading control. Amplification was shown to be in the logarithmic phase.

FIG. 23—Effects of myb28-1 myb29-1 double mutant on glucosinolate accumulation. Homozygous wild-type, homozygous single mutant or homozygous double mutant progeny were measured for foliar and seed glucosinolates by HPLC. 12 independent plants were separately measured per line for the four lines and the data analyzed via ANOVA. Data for 4MSB, 8MSO, total aliphatic and total indolic glucosinolate content are shown. Genotypes with different letters show statistically different glucosinolate levels for the given glucosinolate.

### EXAMPLES

### Materials and Methods

Any methods of the invention not specifically described below may be performed by one of ordinary skill in the art without undue burden in the light of the disclosure herein.

### Sequences

Different annotations of the size of mRNA and coding sequences of MYB28 and MYB29 are found in the databases. The MYB28 mRNA sequence is found in two different versions in the NCBI database (http://www.ncbi.nlm.nih.gov/entrez/query.fcgi?db=Nucleotide). One (NM_{—}125535) encodes a transcript of 1425 bp long and encodes for a protein of 367 amino acids. Another mRNA (NM_{—}180910) is 1805 bp long and is predicted to encode a 288 amino acid protein. Similarly, two different MYB29 mRNA sequences exist—one of 1595 bp (NM_{—}120851) and one of 1292 bp (AF062872)—both are predicted to encode a 337 aa protein. The MYB76 mRNA (NM_{—}120852, DQ446930, AF175992) of 1017 bp is predicted to encode a protein of 339 aa. The two different coding sequences of MYB28 were aligned with the annotated coding sequences of MYB29 and MYB76. It was found that the 288 amino acid protein lacked the crucial R2 and most of the R3 DNA binding domain. Consequently, the 367 amino acid encoding region was regarded as the correct coding sequence and subsequently used in the clonings.

Cloning of At1g65860 and At1g62560 for Heterologous Expression

Total plant RNA was isolated with Trizol (Invitrogen) according to the manufacturer's recommendations. First strand cDNA was synthesized using the iScript™ cDNA synthesis kit (BioRad). At1g65860 CDS and At1g65860 CDS without stop codon was amplified using 1 μL of first strand product using primer combinations 109/110 and 109/111, respectively, in a 50 μL reaction using the Easy-A™ High-Fidelity PCR Cloning Enzyme (Stratagene) following the manufacturer's recommendations. At1g62560 CDS was amplified by the same procedure but using the primer combination 104/105. At1g62560 CDS was cloned into pCR®II-TOPO® (Invitrogen) using the manufactures description.

At1g65860 CDS and At1g65860 CDS without stop codon was cloned into pBAD-TOPO® (Invitrogen) using the manufactures description resulting in an arabinose-inducible expression construct for un-tagged and his-tagged At1g65860.

Sequencing was performed by MWG Biotech (Germany).

Primers were as follows:

| | | | | Size of |
|---|---|---|---|---|
| Primer | | | Used | Product |
| number | Primer name | Primer sequence (5′-3′) | with | (bp) |
| 20 | BamHI-p35Senh-F | AATAACAggatccCTTCGTCAACATGGTGGAGC | 23, 29 | 329, ~3400 |
| 23 | Myb76-p35Senh-R | ttttagtacagtgaacgcttGGAGATATCACATCAATCCACTT | 20 | 329 |
| 24 | p35S-enh-myb28-F | TTGATTGATGTGATATCTCCtttgcaaaatgatagtggagaa | 27 | 3995 |
| 26 | p35S-enh-myb76-F | TTGATTGATGTGATATCTCCaagcgttcactgtactaaaacca | 29 | 3072 |
| 27 | PstI-myb28-R | aataacaCTGCAGttgatgactattatgggcactga | 24 | 3995 |
| 29 | PstI-myb76-R | aataacaCTGCAGtcaacattgggaaattgacaag | 26 | 3072 |
| 41 | PstI-myb29-R | aataacaCTGCAGgtagggatttgtttcttcggagt | 67 | 4135 |
| 49 | pCambia2300-F | GagcggataacaatttcacacaAATAACAgagctcCTTCGTCAA | 55 | ~400 |
| | | CATGGTG | | |
| 54 | SacI-p35Senh-F | GAGCAATAACAgagctcGGAGATATCACATCAA | 55 | 329 |
| 55 | SacI-p35Senh-R | TCCACTT | 54, 66 | 329 |
| 59 | USER-Myb29-R | GGTTTAAUggctcaaactttaaatcaaatggt | 68 | 3823 |
| | | | | 3885 + |
| 60 | USER-nMyb28-F | GGCTTAAUcaatgtaaatgctcggaagtga | 61 | USER |
| | | | | 3885 + |
| 61 | USER-nMyb28-R | GGTTTAUaatgggaagtactactacgaaataagaAATAACAgaa | 60 | USER |
| | | ttcCTTCGTCAACATGGTG | | |
| 66 | EcoRI-35Senh-F | GAGC | 55 | 329 |
| 67 | outsMyb29-F | ccttggttacaatatatgcagcttt | 41 | 4135 |
| | USER-snMyb29-F | | | |
| 68 | short | GGCTTAAUattttcaacgattgcgttgtttggcttaaUATGTCA | 59 | 3823 |
| | | AAGAGACCATATTGTA | | |
| 75 | MYB76 f U | TCggtttaaUTCATAAGAAGTTCTTCTCGTCG | | |
| 76 | MYB76 r U | GA | | |
| 45 | MYB76 f | ATGTCAAAGAGACCATATTGTATC | | |
| 46 | MYB76 r | TCATAAGAAGTTCTTCTCGTCGGA | | |
| 156 | MYB28 f | atgtcaagaaagccatgttgcgtc | | |
| 50 | MYB28 r | TCATATGAAATGCTTTTCAAGCGA | | |
| 157 | MYB28 f U | ggcttaaUatgtcaagaaagccatgttgcgt | | |
| 74 | MYB28 r U | ggtttaaUTCATATGAAATGCTTTTCAAG | | |
| 100 | MYB29 f | ATGTCAAGAAAGCCATGTTG | | |
| 101 | MYB29 r | TCATATGAAGTTCTTGTCGTC | | |
| 71 | MYB29 r U | ggtttaaUTCATATGAAGTTCTTGTCGTC | | |
| 72 | MYB29 f U | ggcttaaUATGTCAAGAAAGCCATGTTG | | |
| 104 | At1g62560 f | ATGGCACCAGCTCAAAACCAAATC | | |
| 105 | At1g62560 r | TCATCTTCCATTTTCGAGGTAATAAGggcttaaUATGGCACCAG | | |
| | | CTCAAAACCAA | | |
| 102 | At1g62560 f U | ATCggtttaaUTCATCTTCCATTTTCGAGGTAA | | |
| 103 | At1g62560 r U | TAAG | | |
| 109 | At1g65860 f | ATGGCACCAACTCAAAACACAATC | | |
| 110 | At1g65860 r | TCATGATTCGAGGAAATAAGAAG | | |
| 111 | At1g65860 r-stop | TGATTCGAGGAAATAAGAAGGAggcttaaUATGGCACCAACTCA | | |
| | | AAACACA | | |
| 107 | At1g65860 f U | ATCggtttaaUTCATGATTCGAGGAAATAAGAA | | |
| 108 | At1g65860 r U | G | | |

Heterologous Expression of At1g65860

At1g65860 pBAD-TOPO constructs and a vector control (empty pBAD-TOPO) were transformed into the E. coli strain TOP10 (Invitrogen). Single colonies were grown overnight in Luria broth (LB) with 100 μg/ml ampicillin. One milliliter of the overnight culture was used to inoculate 100 ml of LB medium supplemented with 100 μg/ml ampicillin and the culture was grown at 37° C., 250 rpm to OD600=0.5, at which time arabinose (final conc. 0.02%) was added. The culture was grown at 28° C., 250 rpm for 16 h.

Following arabinose induction, spheroplasts of E. coli cells were prepared. The culture was chilled on ice, pelleted at 2500 g for 10 min, followed by resuspension by sequential addition of 8.3 ml 200 mM Tris/HCl, pH 7.6, 1.42 g sucrose, 16.7 μL 0.5 mM EDTA, 41.7 μL 0.1M phenylmethylsulfonyl fluoride, 33.3 μL lysozyme (50 mg/ml) and finally 8.31 mL ice-cold water with slow stirring. After 30 min incubation at 4° C. with slow stirring, 166 μL 1M Mg(OAc)₂ was added and membranes were pelleted at 3000 g for 10 min at 4° C. Pellet was resuspended in 1800 μL 10 mM Tris/HCl pH 7.6/14 mM Mg(OAc)₂/60 mM KOAc. The suspension was homogenized in a Potter-Elvehjem followed by the addition of 5 μL RNAse (10 mg/ml) and 5 μL DNAse (5 mg/ml) and slowly stirred for 30 min at 4° C. 235 μL 87% glycerol was added and the spheroplasts were stored at −80° C. for several weeks without loss of activity.

Enzymatic Activity of Heterologously Expressed At1g65860 in E. coli Spheroplasts.

The 100 ul assay solution contained substrate and spheroplasts corresponding to 50 ug total E. coli protein in a 0.1 M Tricine at pH 7.9, 0.25 mM NADPH buffer. The reaction was allowed to proceed for 1 hour at 30° C. and terminated by the addition of 100 μL methanol and centrifuged at 5000 g for 2 min. The supernatant was moved to new tubes followed by lyophilization to dryness and finally redissolved in 50 μL water.

4-methylthiobutyl glucosinolate and desulfo 4-methylthiobutyl glucosinolate were used as substrates with final concentrations in the assay as given in FIG. 3. 4-methylthiobutyl oxime, dihomomethionine, methionine were tested as substrates at 0.1 mM, 1 mM and 10 mM final concentrations.

### Identification and Quantification of FMO Substrates and Products

### Amino Acids

Dihomomethionine (Dawson et al. 1993), L-methionine (Sigma) and L-methionine sulfoxide (Sigma) standards as well as the supernatant from the dihomomethionine and L-methionine assays were derivatized with o-phaldialdehyde (OPA) by the procedure described by Aboulmagd et al (2000). Two minutes after start of derivatization, the sample was injected onto a ZORBAX SB-Aq (4.6×250 mm, RPC₁₈, 5 μm particle size, Agilent) on a Dionex HPLC system consisting of P580 pump/UVD340 S/GINA 50 autosampler.

Buffers used for elution of the OPA derivatives were a follows: A, 50 mM sodium acetate (pH 4.5) and 20% acetonitrile; B, 100% acetonitrile.

The following linear gradients were used: a 15 min gradient from 0% to 80% eluent B, 5 min at 80% eluent B, a 3 min gradient from 80% to 0% eluent B, and a final 3 min at 0% eluent B (25° C., flow 1 ml/min. The OPA derivatives were detected by measuring the fluorescence at 450 nm after excitation at 330 nm using a Dionex RF2000 Fluorescence detector.

### Oximes

4-methylthiobutyl oxime (Dawson et al. 1993) and supernatant from 4-methylthiobutyl assays were injected onto a ZORBAX SB-Aq (4.6×250 mm, RPC₁₈, 5 μm particle size, Agilent) on a Dionex HPLC system consisting of P580 pump/UVD340 S/GINA 50 autosampler.

Compounds were detected at 229 nm and separated utilizing eluents: A, H₂O and B, 100% acetonitrile using the following program. A 5 min gradient from 1.5% to 7% eluent B, 5 min gradient from 7% to 25% eluent B, 4 min gradient from 25% to 80% eluent B, 3 min at 80% eluent B, 3 min gradient from 80% to 99% eluent B, 6 min gradient from 99% to 1.5% eluent B and a final 3 min at 1.5 eluent B.

### Glucosinolates

Detection of 4-methylthiobutyl glucosinolate and 4-methylsulfinylbutyl glucosinolate and supernatant from 4-methylthiobutyl glucosinolate assays were performed as described in Kliebenstein et al (2001) with minor modifications. A 96 well filter plate (Millipore, Eschborn, Germany cat. no. MAHVN 4550) was loaded with 45 μl sephadex A-25* using the Millipore multiscreen column loader (Millipore, ca. no; MACL 09645). 300 μl water was added to the columns and allowed to equilibrate for two hours. The water was removed by applying vacuum on the vacuum manifold (Millipore, Denmark) for 2-4 s. The supernatant was applied to the column and vacuum applied for 2-4 s. The column was washed twice with 150 μl 70% methanol and twice with 150 μl H₂O. 10 μl of sulfatase solution (2.5 mg/ml sulfatase (Sigma E.C. 3.1.6.1)) was added to each column and left to incubate at room temperature over night. The desulfoglucosinolates were eluted with 100 μl H₂O by placing the 96 well column plate on top of a deep well 2 ml 96 well plate in the vacuum manifold.

Detection of desulfo-4-methylthiobutyl glucosinolate and desulfo-4-methylsulfinylbutyl glucosinolate and supernatant from desulfo-4-methylthiobutyl glucosinolate were performed as described for 4-methylthiobutyl glucosinolate and 4-methylsulfinylbutyl glucosinolate with the modification that they were injected directly on to the HPLC without any binding to column material etc.

Compounds were detected at 229 nm and separated utilizing eluents: A, H₂O and B, 100% acetonitrile using the following program. A 5 min gradient from 1.5% to 7% eluent B, 5 min gradient from 7% to 25% eluent B, 4 min gradient from 25% to 80% eluent B, 3 min at 80% eluent B, 2 min gradient from 80% to 35% eluent B, 2 min gradient from 35% to 1.5% eluent B and a final 3 min at 1.5 eluent B.

### Transformation of Arabidopsis and in Planta Overexpression

### Cloning of MYB28, MYB29 and MYB76

Total plant RNA was isolated with Trizol (Invitrogen) according to the manufacturer's recommendations. First strand cDNA was synthesized using the iScript™ cDNA synthesis kit (BioRad). MYB28, MYB29 and MYB76 CDS's were amplified using 1 μL of first strand product with primer combinations 156/50 for MYB28, 100/101 for MYB29 and finally 45/46 for MYB76, in a 50 μL reaction using the Easy-A™ High-Fidelity PCR Cloning Enzyme (Stratagene) following the manufacturer's recommendations. Following PCR amplification MYB28, MYB29 and MYB76 CDS's were cloned into pCR®II-TOPO® (Invitrogen) using the manufactures description. Sequencing was performed by MWG Biotech (Germany).

### Constructs for Constitutive Expression in Planta.

To construct 35S overexpression constructs, PCR was performed with PfuTurbo® Cₓ Hotstart DNA polymerase on the clones mentioned above with the primer combinations 102/103 for At1g62560, 107/108 for At1g65860, 75/76 for MYB 76, 157/74 for MYB28 and finally 71/72 for MYB29. The PCR products were cloned into pCAMBIA230035Su (Noir-Eldin et al. 2006) using the method described in Noir-Eldin et al. 2006.

### Constructs for Endogenous Overexpression in Planta.

The PfuTurbo® Cₓ Hotstart DNA polymerase (Stratagene) was used for PCRs with uracil-containing primers according to the manufacturer's instruction and the Phusion™ High-Fidelity DNA polymerase (Phusion) (Finnzymes, Espoo, Finland) in the remaining reactions according to the manufacturer's instructions.

The 35Senh-Myb76 PCR product was constructed by overlapping PCR. The 35Senh part was amplified from the pCambial302 (GenBank accession no. AF234298) with primers 20 and 23. The Myb76 PCR fragment was amplified from Arabidopsis thaliana ecotype Columbia gDNA with primers 26 and 29. The overlapping PCR was conducted with primers 20 and 29 with the template consisting of a mixture of 2 μl 35Senh PCR reaction and 4 μl Myb76 PCR reaction. The overlapping PCR fragment was cut with BamH I and Pst I and ligated with a BamH I and Pst I-digested pCambia2300 (GenBank accession no. AF234315).

The pCambia2300-35Senh-USER (Sac I)-vector was made by introducing the 35Senh-element into the Sac I sites of the pCambia2300u vector (Nour-Eldin et al. 2006). The template for the 35Senh product was produced by cutting 10 μl pCambia1302 miniprep with 2 units SphI at 37° C. for one hour and subsequently gel purifying the 1930 bp large piece (the other being 8619) which contained the 35S promoter element meant to express GFP. 0.02 μl of this product was used to amplify the 35Senh-PCR product with primers 54 and 55 thereby introducing Sac I sites in both ends of the product. The PCR fragment was cut with Sac I and ligated into the Sac I-cut pCambia2300u vector.

The pCambia2300-35Senh-USER (EcoR I and Sac I) vector was made by introducing the 35Senh-element into the EcoR I and Sac I sites of the pCambia2300u vector (Nour-Eldin et al. 2006). The PCR product was amplified by primers 55 and 66 from a SphI-cut pCambia1302 (see above) thereby introducing a EcoR I and Sac I site in forward and reverse end, respectively, of the product. The pCambia2300u vector was cut with EcoR I and Sac I enzymes and ligated with the likewise cut PCR product.

pCambia2300-35Senh-USER-Myb28: the Myb28 promoter and genomic locus was amplified with primers 24 and 27 from gDNA. Subsequently, 1 μl of this reaction was used to perform a nested PCR with the primers 60 and 61. The PCR product was subsequently cloned into the pCambia2300-35Senh-USER (Sac I)-vector as described in Nour-Eldin et al. 2006.

pCambia2300-35Senh-USER-Myb29: the Myb29 promoter and genomic locus was amplified with primers 67 and 41 from gDNA. Subsequently, 1 μl of this reaction was used to perform a nested PCR with the primers 68 and 59. The PCR product was subsequently cloned into the pCambia2300-35Senh-USER (EcoR I and Sac I)-vector as described in Nour-Eldin et al. 2006.

### Plant Transformation.

The constructs were transformed into Agrobacterium tumefaciens strain C58 (Shen and Forde, 1989) and into Arabidopsis thaliana Col-0 by Agrobacterium tumefaciens strain C58 (Zambryski et al 1983) mediated plant transformation using the floral dip method (Clough and Bent, 1998). Transgenic plants were selected on 50 μg/ml kanamycin ½ MS plates.

Cabbage and oil-seed rape may be transformed by previously described methods (Moloney et al., (1989) Plant Cell Rep. 8, 238-242) likewise pea (Bean et al., (1997) Plant Cell Rep. 16, 513-519), potato (Edwards et al., (1995) Plant J. 8, 283-294) and tobacco (Guerineau et al., (1990) Plant Mol. Biol. 15, 127-136).

### Plant Growth Conditions:

Surface-sterilized seeds were sown on 0.5×MS plates containing 50 μg/ml kanamycin and kept in darkness at 5 degrees for two days before transferal to growth chambers (HEMZ 20/240/S, Heraeus) at a photosynthetic flux of 100 μE at 20° C. and 70% relative humidity at a 16 h photoperiod. After 12-14 days on plates, the plants were transferred to a soil:vermiculite (10:1) mixture wetted with Bactimos L (Garta, Copenhagen, DK).

### Glucosinolate Analyses on Plant Material

A mix of heterozygous and homozygous T2 35Senh-Myb76, 35S:Myb28, 35S:Myb29, 35S:Myb76 as well as Arabidopsis thaliana, ecotype Columbia, were used in one experiments. In a separate experiment, a mix of heterozygous and homozygous T2 35Senh-Myb28 plants, T1 ‘empty vector’ plants (MP16A) and Arabidopsis thaliana, ecotype Columbia, were used.

In another separate experiment a mix of heterozygous and homozygous 35S:At1g62560 and 35S:At1g65860 as well as Arabidopsis thaliana, ecotype Columbia, were used.

Three-four leaves (20-80 mg) were harvested from each plant and the material freeze-dried O/N. After adding one ball bearing to each tube, the tissue was homogenized by shaking at a frequency of 30 s⁻¹ for one min on a Retsch Mixer Mill 303 (Retsch, Haan, Germany). 250 μl of 85% methanol was added to each tube and the entire box vortexed for 30 s. For glucosinolate extraction from seeds (10-20 mg) 250 μl of 85% methanol was added before homogenization. The samples were mixed for two minutes by vortexing. A 96 well filter plates (Millipore, Eschborn, Germany cat. no. MAHVN 4550) was loaded with 45 μl sephadex A-25* using the Millipore multiscreen column loader (Millipore, ca. no. MACL 09645). 300 μl water were added to the columns and allowed to equilibrate for two hours. The water was removed by applying vacuum on the vacuum manifold (Millipore, Denmark) for 2-4 s. Tissue and proteins were pelleted by centrifuging at 2500 g for ten min in Rotanta 460 (Hettich Tuttlingen, Germany). The supernatant was applied to the column and vacuum applied for 2-4 s. The column was washed twice with 150 μl 70% methanol and twice with 150 μl H₂O. 10 μl of sulfatase solution (2.5 mg/ml sulfatase (Sigma E.C. 3.1.6.1)) was added to each column and left to incubate at room temperature over night.

The desulfoglucosinolates were eluted with 100 μl H₂O by placing the 96 well column plate on top of a deep well 2 ml 96 well plate in the vacuum manifold.

The standards were made by applying 100 μl (10 mM pOHBG, 10 mM sinigrin and 1 mM N-MeOH-I3M) to the column and following the procedure above except that the sample was eluted in 200 μl H₂O. A dilution series with 35 standards were made so that the highest amount injected on the Liquid Chromatography-Mass spectrometry (LC-MS) apparatus was 100 nmol pOHBG, 100 nm sinigrin and 10 nm N-MeOH-13M and the lowest was 5.61 pmol, 5.61 pm and 0.561 pmol, respectively.

### LC-MS Analysis

20 μl sample was injected by ASI-100 Automated Sample injector (Dionex, Denmark) and separated on a Zorbax SB-AQ RPC18 column (4.6 mm×250 mm, 5 um) (Agilent Technologies, USA) at a flow rate of 1 ml/min delivered by a P680 HPLC pump (Dionex). Compounds were detected at 229 nm and separated utilizing eluents: A, H₂O and B, 100% acetonitrile using the following program. A 5 min gradient from 1.5% to 7% eluent B, 5 min gradient from 7% to 25% eluent B, 4 min gradient from 25% to 80% eluent B, 3 min at 80% eluent B, 2 min gradient from 80% to 35% eluent B, 2 min gradient from 35% to 1.5% eluent B and a final 3 min at 1.5 eluent B. A STH585 column thermostate (Dionex) kept the column temperature at the set 25° C. The desulfoglucosinolates were detected at 229 nm by a UV-detector equipped with a micro flow cell (UVD340S, Dionex). The mobile phase was split using a T-piece and delivered 20% of the total flow (1 ml/min) to the mass spectrometer. Mass spectrometry was carried out on a single quadrupole Thermo Finnigan Surveyor MSQ equipped with electrospray injection. The electrospray capillary voltage was set at 3 kV, the cone voltage at a constant 75 V and the temperature was 365° C. For ionization 50 μl/min of 250 μM NaCl was added to the flow (after split) using an AXP-MS high pressure pump (Dionex) and the desulfoglucosinolates were detected as [M+Na]⁺ adduct ions.

Desulfoglucosinolates were identified according to masses and earlier experience with retention times (Dan Kliebenstein, University of California-Davis, Department of Plant Sciences, USA) and quantified by the A_{229 nm} response of the standards (sinigrin and N-methoxy-indole glucosinolate). Data was extracted using the program Chromeleon (Dionex).

### HPLC Analysis

The samples were analysed at the same conditions as above but the HPLC consisted of a P580 pump (Dionex), a ASI-100 Automated Sample injector (Dionex, Denmark) and a UV-detector equipped with a standard flow cell (UVD340S, Dionex).

Data was extracted using the program Chromeleon (Dionex).

### ABBREVIATIONS USED IN THE EXAMPLES AND FIGURES

3MSP—3-methylsulfinylpropyl GSL
3MTP—3-methylthiopropyl GSL
4MSB—4-methylsulfinylbutyl GSL
4MTB—4-methylthiobutyl GSL
5MSP—5 methylsulfinylpentyl GSL
5MTP—5-methylthiopentyl GSL
6MSH—6-methylsulfinylhexyl GSL
6MTH—6-methylthiohexyl GSL
7MSH—7-methylsulfinylheptyl GSL
7MTH—7-methylthioheptyl GSL
8MSO—8-methylsulfinylocytyl GSL
8MTO—8-methylthiooctyl GSL
I3m—indol-3-yl methyl GSL
4MOHI3M—4-methoxy-indol-3-yl methyl GSL
NMOHI3M—N-methoxy-indol-3-yl methyl GSL

### DNA Extraction and Genotyping of T-DNA Insertion Mutants

Total DNA was extracted essentially as described in (Lukowitz et al., 2000). T-DNA insertions in At5g61420 (line SALK_{—}136312=myb28-1), At5g07690 (lines GABI_{—}868E02=myb29-1 and SM.34316=myb29-2) and At5g07700 (lines SALK_{—}096949=myb76-1 and SALK_{—}055242=myb76-2) were confirmed by PCR. myb28-1 had a T-DNA insertion in the 5′UTR region of the gene, 182 bp upstream of the start codon. The T-DNAs of myb29-1 and myb29-2 are positioned in the third exon 730 bp upstream of the stop codon and in the 5′UTR 40 bp upstream the start codon, respectively. The T-DNAs of myb76-1 and myb76-2 are situated respectively, 99 bp downstream the ATG in the first exon and 194 downstream the ATG in the first intron. Two separate PCR reactions were carried out to identify the position of the insertion site and the zygosity of the plants. Forward and reverse primers were designed according to the SIGnAL T-DNA verification primer design tool (http://signal.salk.edu/tdnaprimers.2.html) for the SALK lines and with Primer3 (http://frodo.wi.mit.edu/cgi-bin/primer3/primer3_www.cgi) for the GABI line. The gene specific primers were used in combination with left border primers (LBa1 for SALK lines or 8409 LB for GABI lines and Spm32 for the SM-line) to verify the presence and orientation of the T-DNAs. Primer sequences used for genotyping are summarized in Supplemental Table 18. Eppendorf HotMaster Taq DNA Polymerase (Hotmaster) (Eppendorf, AG, Hamburg, Germany) was used in a 20 μl reaction using 1 unit enzyme, 187.5 μM dNTP, buffer 1:10 and 187.5 μM of each primer and DNA as template. The PCR program was as follows: Denaturation at 94° C. for 3 min, 35 cycles of denaturation at 94° C. for 30 s, annealing at 56° C. for 30 s, extension at 65° C. for 1.15 min and finally extension at 65° C. for 3 min.

myb28-1 myb29-1 Double Mutant Construction

To construct the double mutant, myb28-1 myb29-1, the homozygous myb28-1 and myb29-1 were crossed with each other. The F₁ plant was self-fertilized and progeny in the F₂ generation was genotyped by PCR (see above).

### RT-PCR on Knockouts and Wild-Type

Leaves from homozygous myb28-1 single knockout, homozygous myb28-1 myb29-1 double knockout and Arabidopsis Columbia wild-type plants (all derived from a segregating myb28-1 myb29-1 F2 plants) were harvested 25 days after germination. Leaves from plants homozygous for the absence or presence of the myb29-1, myb29-2, myb76-1 and myb76-2 allele were harvested 23 days after germination. RNA was extracted with Trizol reagent (Invitrogen, Carlsbad, Calif.) according to the manufacturer's instructions. The samples were DNAse treated with 2 units DNA Free™ (Ambion, Cambridgeshire, Great Britain) according to the manufacturer's instructions. One μg of total RNA was reverse transcribed using the iScript cDNA Synthesis Kit (Biorad, Hercules, Calif.). The primers used for RT-PCR are listed in Supplemental Table 18. PCR was performed with Eppendorf HotMaster Taq DNA Polymerase (Hotmaster) (Eppendorf, AG, Hamburg, Germany) in a 20 μl reaction using 1 unit enzyme, 187.5 μM dNTP, buffer 1:10 and 187.5 μM of each primer and cDNA as template. The PCR program was as follows: Denaturation at 94° C. for 3 min, 22-35 cycles of denaturation at 94° C. for 30 s, annealing at 53-56° C. for 30 s, extension at 65° C. for 0.45-1.15 min and finally extension at 65° C. for 3 min. Microarray Analysis of MYB Over-expression

Plants for the various genotypes were grown as previously described. At 25 days post germination, a fully-expanded mature leaf was harvested, weighed and analyzed for total aliphatic glucosinolate content via HPLC. The remaining plant material was collected, flash frozen and total RNA extracted via RNeasy columns (Qiagen, Valencia, Calif., USA). Two independent plants were combined to provide sufficient starting material for a single RNA extraction. Two independent samples were obtained per transgenic line with two different transgenic lines per 35S:MYB transgene, thus providing four-fold replication. Six wild-type Col-0 RNA samples were obtained. This provided a total of 18 independent microarrays. Labeled cRNA was prepared and hybridized, according to the manufacturer's guidelines (Affymetrix, Santa Clara, Calif., USA), to whole genome Affymetrix ATH1 GeneChip microarrays, containing 22,746 Arabidopsis transcripts. The GeneChips were scanned with an Affymetrix GeneArray 2500 Scanner and data acquired via the Microarray Suite software MAS 5.0 at the Functional Genomics Laboratory (University of California Berkeley). RMA normalization was used to obtain gene expression levels for all data analyses (Irizarry et al., 2003).

### Microarray Statistical Analysis

The gene expression data was first analyzed via a network/biosynthetic pathway ANOVA approach utilizing the general linear model within SAS (Kliebenstein et al., 2006b). Sulfur utilization biosynthetic pathways were obtained from AraCyc v3.4 (http://www.arabidopsis.org/biocyc/) and modified to better organize the pathways based on metabolites of importance for glucosinolate synthesis. Transcription factor networks for aliphatic and indole glucosinolates were added based on this research and previously published research (Celenza et al., 2005; Levy et al., 2005; Skirycz et al., 2006). Each selected, independent 35S:MYB line was tested against the wild-type data in an independent ANOVA. For the ANOVA, the genes were nested factors within the higher order pathway. Additionally, the two independent lines per 35S:MYB transgene were nested factors [Transgene(Genotype)] within the Genotype term (wild-type versus 35S:MYB). This allowed us to test for effects due to the transgene versus the independent transgenic line. Each pathway was then tested within the model for a difference between the wild-type and 35S:MYB lines using an F-test. Additionally, we tested each aliphatic glucosinolate biosynthetic and transcription factor gene for altered transcript accumulation. These individual gene tests were also done within the confines of the model using an F-test to test for a mean separation between wild-type and the 35S:MYB line. All P values for these genes were significant after a FDR adjustment within the confines of this pathway ANOVA utilizing a pre-defined subset of genes meant to address a specific question about sulfur-utilization and glucosinolate biosynthetic pathways.

Next, the gene expression data was analyzed via individual gene ANOVA for each transcript. This was done by conducting ANOVA on each gene using the two independent transgenic lines per 35S:MYB transgene as nested factors [Transgene(Genotype)] within the Genotype (WT versus 35S:MYB transgene) effect. The ANOVA calculations were programmed into Microsoft Excel to obtain all appropriate Sums-of-Squares and to obtain the F values for the effect of the Genotype (wild-type versus 35S:MYB transgene) and Transgene(Genotype) effects for each gene. The nominal P values for both terms are presented as well as the P values for the Genotype (wild-type versus 35S:MYB transgene) effect that are significant after a FDR adjustment to the 0.05 level (Benjamini and Hochberg, 1995).

### Example 1

### the identification of candidate genes for catalyzing the conversion from 4-methylthiobutyl glucosinolate to 4-methylsulphinyl glucosinolate

The “Transcript Co-response single gene query” of CSB-DB—(a comprehensive systems-biology database) (http://csbdb.mpimp-golm.mpg.de/index.html) were used to identify genes co-expressing with the genes in the biosynthesis of aliphatic GSLs (including At4g13770, At1g16410 and At1g16400). Two flavin-containing monooxygenases (At1g65860 and At1g62560) were among the genes that were identified, and these genes were also inside a 17cM QTL for conversion of methylthioalkyl to methylsulfinylalkyl GSL (Kliebenstein et al., Plant Physiology, 126, 811-825, 2001). Since catalysis of this type of reaction is consistent with FMO activity they were elected for characterisation.

### Example 2

### Enzymatic Activity Of Heterologously Expressed FMOs

FIG. 3 shows the enzymatic activity of heterologously expressed At1g65860 in E. coli spheroplasts. The results clearly show the production of 4MSB from 4MTB by the transformed microorganisms.

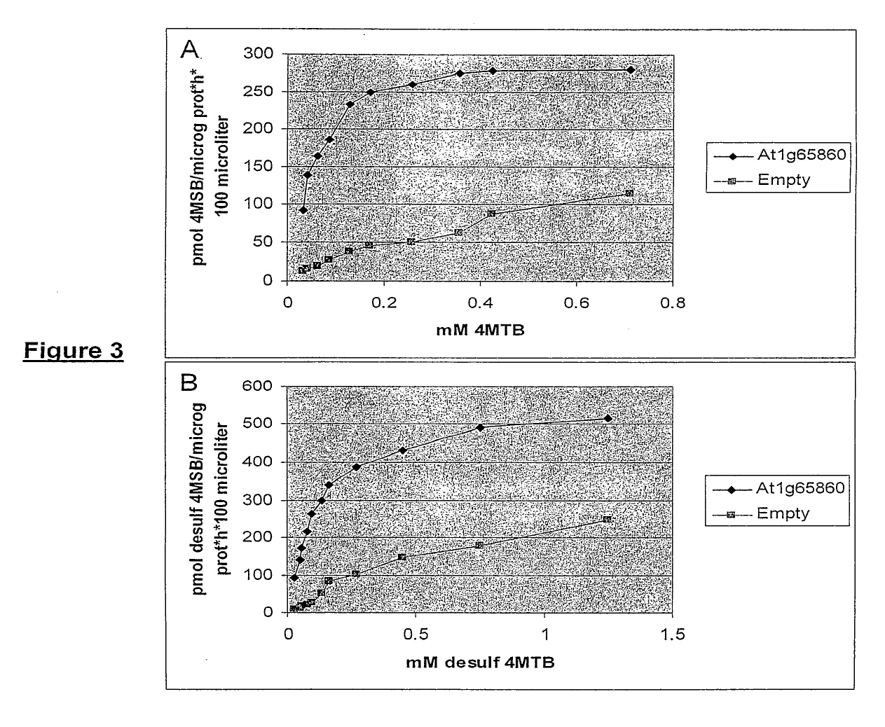

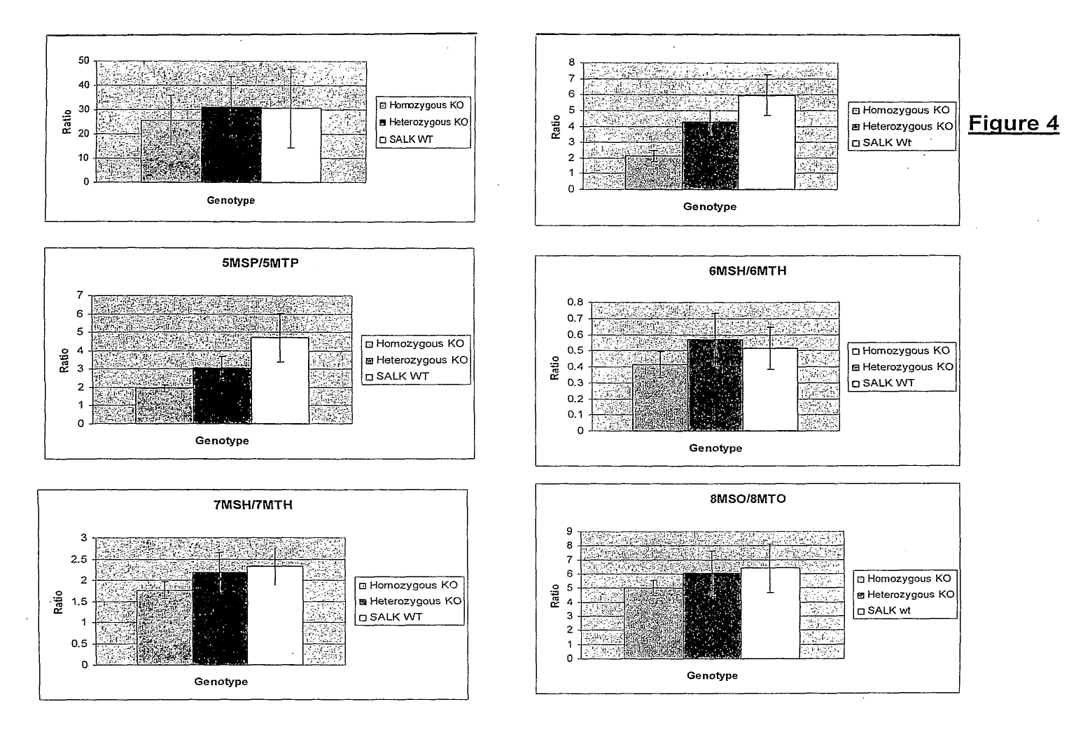

FIG. 4 shows the ratios of sulphinyl/thio GSLs for each specific chain length in Arabidopsis thaliana offspring from a heterozygous segregating knock out in At1g65860 (Salk line 079493). The results clearly show that the FMO encoded by At1g65860 is capable of converting 4- and 5-MTB into 4- and 5-MSB. It is believed that other homologues may have different specificities.

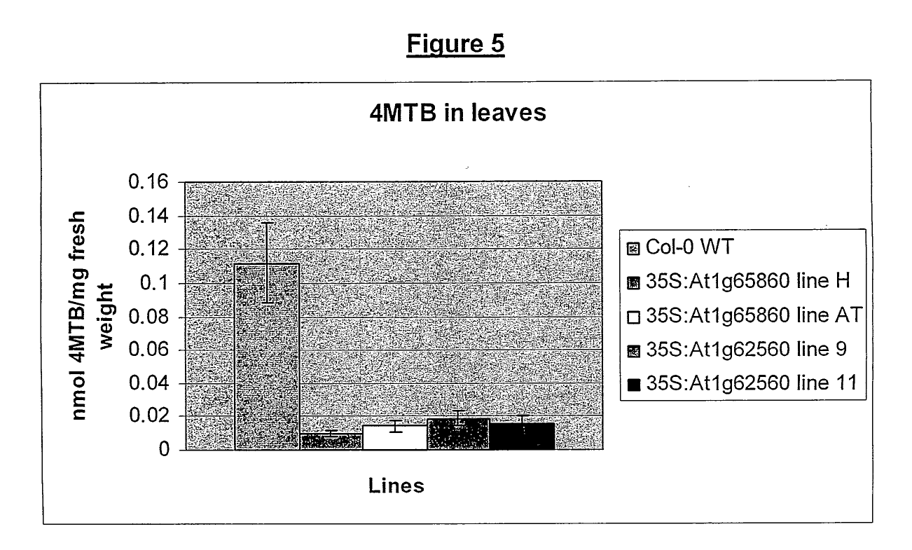

FIG. 5 shows 4MTB levels in leaves from wild type and transgenic At1g65860 and At1g62560 overexpression lines. The results clearly show that the FMO encoded by these genes catalyse the conversion of 4MTB to 4MSB in leaves.

Table 1a shows the GS-OX activity of the At1g65860 T-DNA knock-out mutant. Seeds and leaves of plants were analyzed for GSL content. All plants were segregants derived from a parental line heterozygous for the T-DNA knock-out allele. MT to (MS+MT) represents the ratio of methylthioalkyl GSL to the sum of methylthioalkyl plus methylsulfinylalkyl GSLs for the given GSL class and is an estimation of in planta GS-OX activity. The mean value (mean) and the standard error (SE) of the mean per group is given. P is the P value for GSL differences between the two genotypes as determined by ANOVA. NS indicates non-significant P values (P>0.05).

Table 1b shows the GS-OX activity in At1g65860 over-expression lines. Leaves at 24-day-post-germination and mature seeds from seven individuals from two independent 35S::FMO_{GS-OX1} lines and wildtype were analyzed for GSL content. MT to (MS+MT) represents the ratio of methylthioalkyl to the sum of methylthioalkyl plus methylsulfinylalkyl GSL for the given GSL class and is an estimation of in planta GS-OX activity. The mean value (mean) and standard error (SE) of the mean per group is given. A nested ANOVA was used to test for variation between the independent transgenic lines and between the presence of the transgene and wildtype. P_{Gene} gives the P value for differences between the two genotypes, wild-type versus 35S::FMO_{GS-OX1} over-expression lines. There was no significant difference between the independent transgenic lines for any GSL variable and as such, they were pooled. NS is for non-significant P values (P>0.05). ND indicates that the given GSL was not detected in that genotype. No statistical analyses were conducted on GSLs having one or more ND.

TABLE 1a

| | Leaf Tissue | | | | | Seed Tissue | | | | |
|---|---|---|---|---|---|---|---|---|---|---|
| | Homozygous | | Homozygous | | | Homozygous | | Homozygous | | |
| | knock-out | | wild-type | | | knock-out | | wild-type | | |
| MT to (MS + MT) | Mean | SE | Mean | SE | P = | Mean | SE | Mean | SE | P = |
| propyl GSL (C3) | 0.05 | 0.01 | 0.05 | 0.01 | NS | 0.01 | 0.00 | 0.01 | 0.00 | NS |
| butyl GSL (C4) | 0.32 | 0.02 | 0.15 | 0.01 | <0.001 | 0.69 | 0.01 | 0.68 | 0.01 | NS |
| pentyl GSL (C5) | 0.35 | 0.02 | 0.18 | 0.02 | <0.001 | 0.94 | 0.00 | 0.94 | 0.01 | NS |
| hexyl GSL (C6) | 0.71 | 0.03 | 0.66 | 0.02 | NS | 0.46 | 0.03 | 0.47 | 0.02 | NS |
| heptyl GSL (C7) | 0.37 | 0.01 | 0.27 | 0.02 | 0.016 | 0.75 | 0.01 | 0.77 | 0.01 | NS |
| octyl GSL (C8) | 0.16 | 0.01 | 0.13 | 0.01 | NS | 0.42 | 0.01 | 0.43 | 0.01 | NS |

TABLE 1b

| | Leaf Tissue | | | | | Seed Tissue | | | | |
|---|---|---|---|---|---|---|---|---|---|---|
| | 35S::FMO_{GS-OX1} | | Col-0 | | | 35S::FMO_{GS-OX1} | | Col-0 | | |
| MT to (MS + MT) | Mean | SE | Mean | SE | P_{Gene} = | Mean | SE | Mean | SE | P_{Gene} = |
| propyl GSL (C3) | 0.05 | 0.00 | 0.11 | 0.01 | <0.001 | 0.01 | 0.00 | 0.03 | 0.00 | <0.001 |
| butyl GSL (C4) | 0.01 | 0.00 | 0.16 | 0.01 | <0.001 | 0.23 | 0.01 | 0.75 | 0.01 | <0.001 |
| pentyl GSL (C5) | ND | ND | 0.24 | 0.02 | | 0.26 | 0.02 | 0.83 | 0.02 | <0.001 |
| hexyl GSL (C6) | ND | ND | 0.33 | 0.02 | | 0.05 | 0.01 | 0.33 | 0.03 | <0.001 |
| heptyl GSL (C7) | 0.02 | 0.00 | 0.21 | 0.02 | <0.001 | 0.20 | 0.01 | 0.63 | 0.02 | <0.001 |
| octyl GSL (C8) | 0.03 | 0.00 | 0.13 | 0.01 | <0.001 | 0.10 | 0.01 | 0.33 | 0.01 | <0.001 |

### Example 3

### Activity of the FMOs Against Other Substrates

In addition to 4MTB and desulfo-4MTB (see above) the possibility that the oxygenation of the sulfur might take place at an earlier step in the GSL biosynthesis was also tested. Substrates tested were methionine, one chain-elongated methionine (dihomomethionine) and 4-methylthiobutyl oxime. No oxygenation on the sulfur of any of these was observed suggesting that oxygenation takes place on the intact GSL or on the desulfo-methylthioalkyl-GSL in planta.

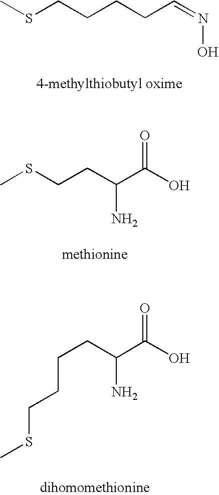

### Example 4

### Identification of Homologues

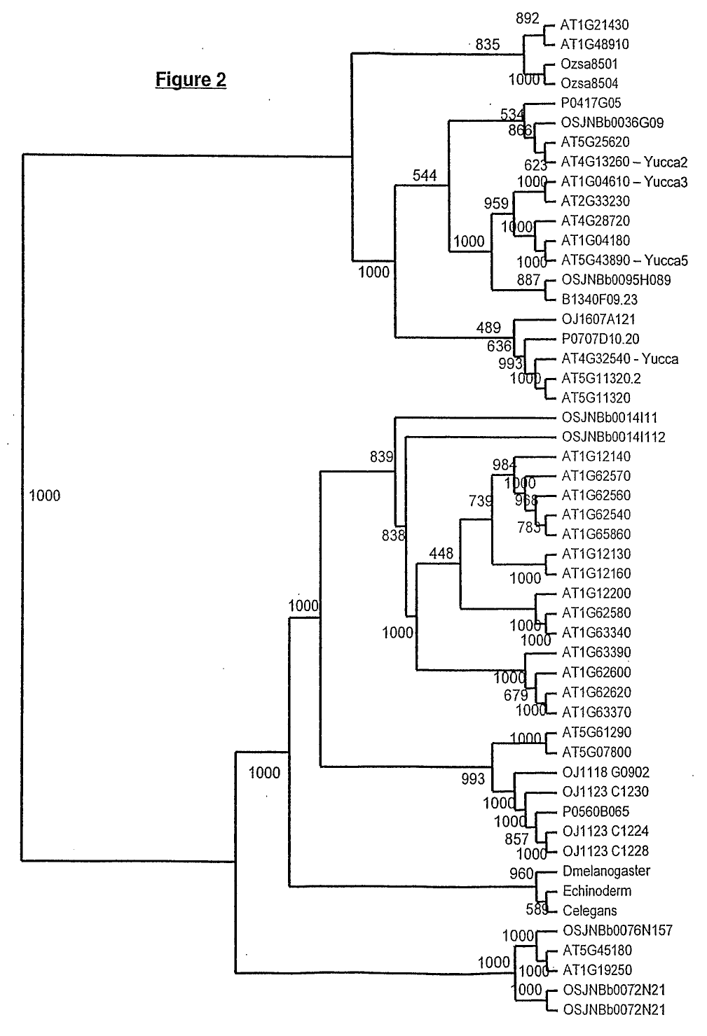

FIG. 2 shows a phylogenetic analysis of protein sequences for the complete genomic complement of all flavin-monooxygenases within Arabidopsis thaliana and Oryza sativa.

At1g62570 and At1g62540 are part of a sub-cluster with At1g62560 and At1g65860 (and At1g12140) and are therefore believed to catalyse the production of sulphinyl-alkyl-GSLs.

Table 1c shows the level of identity and similarity between these various protein sequences.

Identity and similarity were determined using BLASTP analysis with full-length amino acid sequences for the proteins (derived from the DNA sequences in silico) as described via www.ncbi.nim.nih.gov. Analysis was done at http://www.ncbi.nim.nih.gov/BLAST/Genome/PlantBlast.shtml?10 using the BLASTP algorithm against the Arabidopsis protein complement with the following settings.
G Cost to open a gap [Integer]
default=11
E Cost to extend a gap [Integer]
default=1
e Expectation value (E) [Real]
default=10.0

TABLE 1c

| | At1g65860 | At1g62540 | At1g62560 | at1g62570 | at1g12140 | at1g12130 | at1g12160 |
|---|---|---|---|---|---|---|---|
| Identity | | | | | | | |
| At1g65860 | | | | | | | |
| At1g62540 | 81 | | | | | | |
| At1g62560 | 77 | 79 | | | | | |
| at1g62570 | 74 | 76 | 72 | | | | |
| at1g12140 | 68 | 70 | 68 | 70 | | | |
| at1g12130 | 60 | 60 | 60 | 60 | | | |
| at1g12160 | 63 | 63 | 63 | 63 | | | |
| Similarity | | | | | | | |
| At1g65860 | | | | | | | |
| At1g62540 | 89 | | | | | | |
| At1g62560 | 87 | 89 | | | | | |
| at1g62570 | 85 | 89 | 85 | | | | |
| at1g12140 | 80 | 82 | 82 | 81 | | | |
| at1g12130 | 75 | 76 | 76 | 74 | | | |
| at1g12160 | 76 | 78 | 76 | 77 | | | |

In addition to the Arabidopsis homologous FMO-encoding genes catalyzing the oxidation of a methylthioalkyl GSL (or desulfo-methylthioalkyl-GSL) to the corresponding methylsulfinylalkyl GSL similar genes may be identified which catalyze the oxidation of a methylthioalkyl GSL (or desulfo-methylthioalkyl-GSL) to the corresponding methylsulfinylalkyl GSL as described herein. Probes based upon the highly conserved regions may be used to obtain this gene from genomic or cDNA libraries of Capparales.

Conserved regions of FMO-encoding genes are amplified using primers as indicated above and the amplified PCR products used to probe to select cDNA clones from Arabidopsis cDNA. Selected clones are sequenced to check homology at the nucleotide level and predicted amino acid sequence of transcribed regions with FMO-encoding genes.

### Example 5

### Down-Regulation of FMO-Encoding Genes Using Antisense Constructs

Full-length and partial length antisense cDNA constructs are produced in which clones containing selected parts of the transcribed nucleotide sequence are engineered into suitable vectors in reverse orientation, driven by a heterologous promoter.

Arabidopsis ecotypes Columbia and Landsberg erecta are transformed via Agrobacterium-mediated transformation.

The plants are analysed using HPLC and found to have altered glucosinolate composition.

### Example 6

### Use of FMO-Encoding Genes as a Marker for Marker-Assisted Breeding Programmes

A complete or part of FMO-encoding gene nucleotide sequence is used as a DNA probe to identify restriction fragment length polymorphisms or other markers occurring between plant breeding lines of Brassica and other GSL producing taxa, which possess different FMO-encoding alleles using conventional sequence analysis techniques—see e.g. Sorrells & Wilson (1997) Crop Science 37: 691-697.

A complete FMO-encoding gene nucleotide sequence or part thereof may be used to identify the homologous genomic sequence within various Caparales species as discussed above, and these may likewise be used to generate markers for the relevant species.

Primers are designed to amplify PCR products of different sizes from plant breeding lines containing different alleles. CAPS markers are developed by restricting amplified PCR products. In order to ensure there is no recombination within the relevant genes during crossing, typically a marker within the gene as well as two markers flanking each side of the gene will be assessed.

The markers are used in Brassica breeding programmes aimed at manipulating GSL content of the plants. These DNA markers are then used to rapidly screen progeny from a number of diverse breeding designs, e.g. backcrosses, inter-crosses, recombinant inbred lines, for their genotype surrounding the GS-OX loci. This may in particular be done in lines that appear to differ for GS-OX efficiency due to a polymorphism at the FMO capable of converting methylthioalkyl GSL (or desulfo-methylthioalkyl-GSL) to the corresponding methylsulfinylalkyl GSL. The use of DNA markers within and linked to the FMO-encoding genes allows the rapid identification of individuals with the desired genotype without requiring phenotyping.

The invention provides genetic combinations which 1.) exhibit elevated levels of 4-methylsulfinylbutyl glucosinolate and/or 3-methylsulfinylpropyl glucosinolate and 2.) exhibit high activity of the GS-OX allele which encodes an FMO capable of converting methylthioalkyl GSL (or desulfo-methylthioalkyl-GSL) to the corresponding methylsulfinylalkyl GSL and 3.) suitable myrosinase activity capable of producing isothiocyanate derivatives of said GSLs.

### Example 7

### Identification of Candidate Genes for Regulation of Aliphatic GSLs

A search was performed of the “Transcript Co-response single gene query” (Max Planck Institute of Molecular Biology 2005) of “CSB-DB—a comprehensive systems-biology database” for genes co-expressing with the genes in the biosynthesis of aliphatic GSLs.

Two Myb transcription factors were among the genes that were identified, namely Myb28 (At5g61420) and Myb29 (At5g07690). Correspondingly, when querying for genes co-expressing with these two transcription factors, many of the high-scoring hits were aliphatic GSL biosynthetic enzymes.

When subjected to WU-BLASTS-2 (www.arabidopsis.org) another Myb transcription factor, Myb76 (At5g07700), was revealed to have 70% nucleotide identity to Myb29 in the CDS. Furthermore, the two genes were adjacent on the chromosome indicating gene duplication of an ancestor.

TABLE 1d

| | | | | MYB34/ | | |
|---|---|---|---|---|---|---|
| | MYB28 | MYB29 | MYB76 | ATR1 | MYB51 | MYB122 |
| MYB28 | | 59 | 57 | 48 | 38 | 47 |
| MYB29 | 71 | | 64 | 49 | 43 | 51 |
| MYB76 | 69 | 71 | | 42 | 48 | 45 |
| MYB34 | 60 | 61 | 59 | | 44 | 41 |
| MYB51 | 57 | 57 | 60 | 56 | | 57 |
| MYB122 | 64 | 64 | 58 | 58 | 68 | |

BLAST settings were as described in Table 1c. The top right is the percent identity while the bottom left is the percent similarity.

TABLE 1e

| p value | MYB28 | MYB29 | MYB76 | MYB34/ATR1 | MYB51 | MYB122 |
|---|---|---|---|---|---|---|
| MYB28 | | 2.0E−111 | 3.0E−109 | 3.0E−59 | 1.0E−61 | 9.0E−60 |
| MYB29 | | | 1.0E−114 | 5.0E−57 | 1.0E−60 | 3.0E−59 |
| MYB76 | | | | 5.0E−60 | 4.0E−56 | 1.0E−55 |
| MYB34 | | | | | 6.0E−67 | 2.0E−65 |
| MYB51 | | | | | | 7.0E−102 |
| MYB122 | | | | | | |

Table 1e shows the Blast p value for statistical significance between these MYB's, as against the complete Arabidopsis genome.

The positions in the genome of the MYB genes was consistent with the presence of QTLs for aliphatic GSLs in recombinant inbred lines of the Arabidopsis ecotypes, Landsberg and Cape Verde Islands (Kliebenstein et al. 2001a).

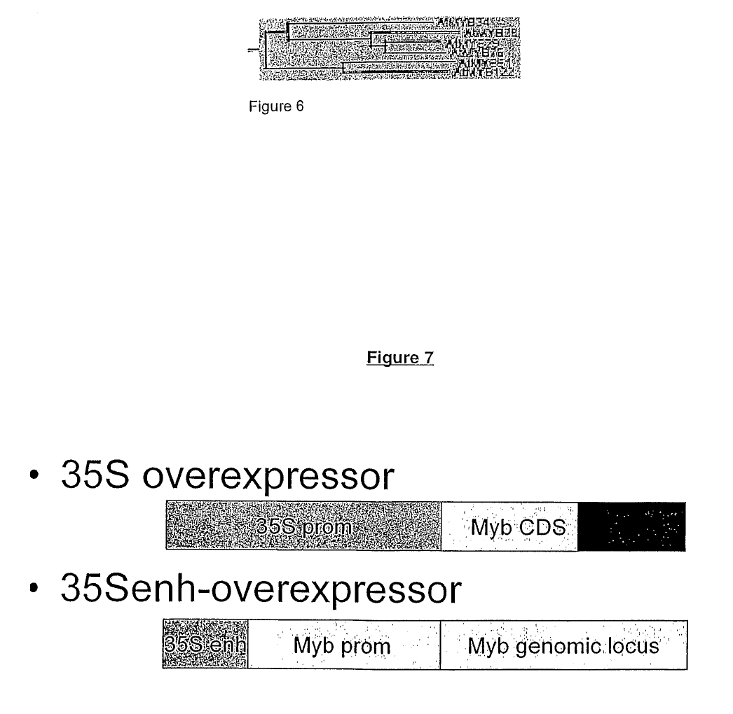

ATR1, another Myb transcription factor, has already been implicated in the regulation of indole GSLs (Celenza et al. 2005). ATR1 is part of a cluster in the Myb transcription family tree where Myb28, Myb29 and Myb76 are also present (FIG. 6).

### Example 8

### Making of Constructs

Overexpression constructs were made for MYB28, MYB29 and MYB76 in order to validate the effects of the genes on GSL levels in planta. The CDS of the three genes was cloned into a vector containing the highly constitutive 35S promoter from Cauliflower Mosaic virus. Transgenic lines obtained from transforming with these overexpressor constructs will be referred to as 35S:Myb28, 35S:Myb29 and 35S:Myb76, respectively. Furthermore, the promoter, along with the genomic locus (encompassing the transcribed region of the gene) were cloned behind one copy of the 35Senhancer, potentially giving endogenous overexpression of the genes. The gene piece cloned encompassed approximately 2 kb promoter upstream the 5′ UTR as well as the 3′-UTR (the 5′UTR and 3′UTR as defined by ‘Sequence viewer’ at www.arabidopsis.org) of the gene. The resulting Myb28, Myb29, Myb76 sequence pieces contained 1896 bp, 2000 bp and 1781 bp, respectively, upstream of the 5′UTR. Likewise, downstream the 3′UTR 44, 43 and 35 bp, respectively, were included in the Myb28, Myb29 and Myb76 sequence pieces.

The sequence of the 35Senhancer was amplified based on an alignment of the 4× 35Senh in the activation tag (Weigel et al. 2000) with the 35S promoter in the pCambial302. The repetitive element was amplified. Both the transgenic lines obtained from transformation with the enhancer constructs will be referred to as 35Senh-Myb28, 35Senh-Myb29 and 35Senh-Myb76.

FIG. 7 shows an overview of the overexpression constructs used.

### Example 9

### Analysis of Arabidopsis Transformants Overexpressing the Myb Transcription Factors

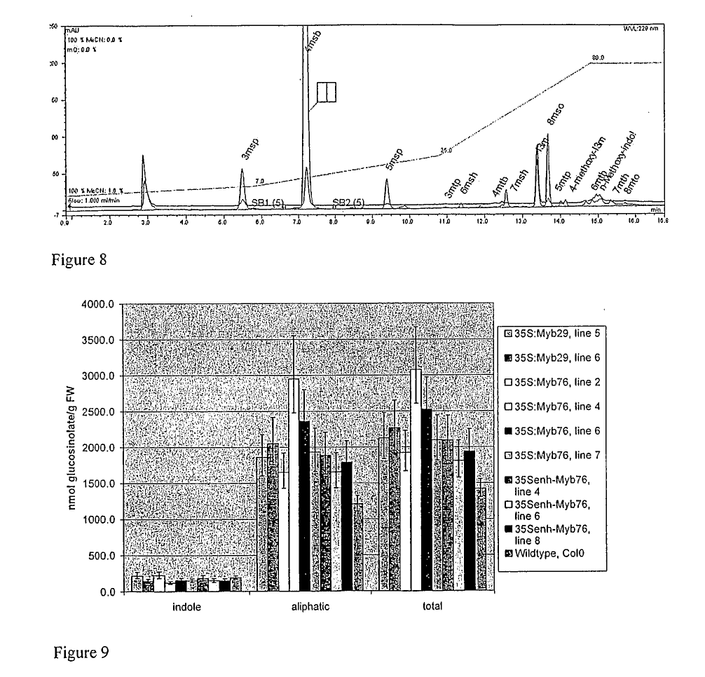

FIG. 8 shows an HPLC chromatogram of desulfoGSL profiles of 35S:Myb76, line 6 (blue line) and wildtype Col-O (black line).

A mixture of homozygous and heterozygous plants from the T2 generations of seeds transformed with the 35S:Myb28, 35S:Myb29, 35S:Myb76 and 35Senh-Myb76 constructs were used for the experiment. Twelve lines of 35S:Myb28, 35S:Myb76 and 35Senh-Myb76 and seven lines of 35S:Myb29 were sown out in six replicates with wildtype plants in a completely randomized design. Leaves of 22 days old plants were harvested and analyzed for contents of GSLs by LC-MS.

None of the lines exhibited any apparent visual phenotype, neither at harvest nor later in their development (data not shown).

FIG. 9 shows the effect of overexpression of Myb29 and MYB76 on indole and aliphatic GSL levels in Arabidopsis. The results are listed in Table 2

Table 2. Individual GSLs in transgenic lines and wildtype. Quantities of GSLs are in nmol/g FW and are the mean from extraction of 5-6 individual plants (transgenic lines) and 14 (wildtype). The relative change in levels of GSLs in comparison to wildtype is stated in parenthesis. The abbreviations can be found in the abbreviation list.

TABLE 2

| | 3MSP | | 3MTP | | 4MSB | | 4MTB | | 5MSP | | 5MTP | |
|---|---|---|---|---|---|---|---|---|---|---|---|---|
| 35S:Myb29, | 145.7 | (151%) | 4.9 | (74%) | 1277.6 | (190%) | 41.4 | (38%) | 59.0 | (206%) | 3.4 | (36%) |
| line 5 | | | | | | | | | | | | |
| 35S:Myb29, | 178.8 | (186%) | 6.0 | (90%) | 1398.1 | (208%) | 34.1 | (31%) | 71.7 | (250%) | 8.0 | (87%) |
| line 6 | | | | | | | | | | | | |
| 35S:Myb76, | 119.7 | (124%) | 6.3 | (94%) | 1018.2 | (151%) | 18.0 | (16%) | 52.1 | (182%) | 7.7 | (84%) |
| line 2 | | | | | | | | | | | | |
| 35S:Myb76, | 222.0 | (231%) | 6.5 | (97%) | 2178.0 | (324%) | 18.8 | (17%) | 118.4 | (413%) | 2.3 | (25%) |
| line 4 | | | | | | | | | | | | |
| 35S:Myb76, | 205.8 | (214%) | 6.2 | (93%) | 1668.1 | (248%) | 12.3 | (11%) | 87.3 | (305%) | 4.1 | (45%) |
| line 6 | | | | | | | | | | | | |
| 35S:Myb76, | 150.4 | (156%) | 4.8 | (71%) | 1306.8 | (194%) | 11.9 | (11%) | 65.4 | (228%) | 4.1 | (45%) |
| line 7 | | | | | | | | | | | | |
| 35Senh- | 149.7 | (156%) | 4.5 | (67%) | 1312.9 | (195%) | 27.4 | (25%) | 53.4 | (186%) | 7.5 | (81%) |
| Myb76, line 4 | | | | | | | | | | | | |
| 35Senh- | 128.9 | (134%) | 5.3 | (80%) | 1158.1 | (172%) | 15.4 | (14%) | 54.2 | (189%) | 5.7 | (62%) |
| Myb76, line 6 | | | | | | | | | | | | |
| 35Senh.Myb76, | 152.7 | (159%) | 5.2 | (78%) | 1275.6 | (190%) | 17.5 | (16%) | 54.3 | (190%) | 3.6 | (39%) |
| line 8 | | | | | | | | | | | | |
| Wildtype, Col0 | 96.2 | (100%) | 6.7 | (100%) | 672.1 | (100%) | 109.7 | (100%) | 28.7 | (100%) | 9.2 | 100%) |

| | 6MSH | | 6MTH | | 7MSH | | 7MTH | | 8MSO | | 8MTO | |
|---|---|---|---|---|---|---|---|---|---|---|---|---|
| 35S:Myb29, | 6.6 | (156%) | 17.7 | (37%) | 19.5 | (116%) | 13.0 | (98%) | 154.2 | (173%) | 19.7 | (91%) |
| line 5 | | | | | | | | | | | | |
| 35S:Myb29, | 12.8 | (301%) | 21.0 | (43%) | 11.1 | (66%) | 13.4 | (101%) | 146.7 | (164%) | 13.2 | (61%) |
| line 6 | | | | | | | | | | | | |
| 35S:Myb76, | 10.2 | (240%) | 109.9 | (227%) | 36.0 | (213%) | 15.9 | (120%) | 150.5 | (169%) | 20.2 | (93%) |
| line 2 | | | | | | | | | | | | |
| 35S:Myb76, | 17.4 | (408%) | 66.6 | (138%) | 18.0 | (107%) | 16.5 | (124%) | 197.3 | (221%) | 20.3 | (93%) |
| line 4 | | | | | | | | | | | | |
| 35S:Myb76, | 14.5 | (340%) | 62.1 | (129%) | 49.0 | (291%) | 9.3 | (70%) | 198.1 | (222%) | 9.8 | (45%) |
| line 6 | | | | | | | | | | | | |
| 35S:Myb76, | 11.0 | (259%) | 87.5 | (181%) | 36.5 | (217%) | 14.8 | (112%) | 149.9 | (168%) | 19.0 | (88%) |
| line 7 | | | | | | | | | | | | |
| 35Senh- | 5.2 | (121%) | 45.3 | (94%) | 23.5 | (140%) | 14.4 | (109%) | 125.0 | (140%) | 21.9 | (101%) |
| Myb76, line 4 | | | | | | | | | | | | |
| 35Senh- | 5.6 | (132%) | 101.6 | (210%) | 16.9 | (100%) | 16.0 | (120%) | 75.0 | (84%) | 18.3 | (84%) |
| Myb76, line 6 | | | | | | | | | | | | |
| 35Senh.Myb76, | 5.1 | (120%) | 44.0 | (91%) | 21.9 | (130%) | 8.0 | (60%) | 135.1 | (151%) | 13.2 | (61%) |
| line 8 | | | | | | | | | | | | |
| Wildtype, Col0 | 4.3 | (100%) | 48.3 | (100%) | 16.9 | (100%) | 13.3 | (100%) | 89.2 | (100%) | 21.7 | (100%) |

| | I3M | | 4MeOHI3M | | NMeOHI3M | | indole | | Aliphatic | | Total | |
|---|---|---|---|---|---|---|---|---|---|---|---|---|
| 35S:Myb29, | 167.3 | (102%) | 24.4 | (171%) | 4.9 | (650%) | 218.1 | (114%) | 1857.9 | (154%) | 2123.8 | (150%) |
| line 5 | | | | | | | | | | | | |
| 35S:Myb29, | 116.1 | (71%) | 22.6 | (159%) | 0.4 | (57%) | 145.0 | (76%) | 2051.4 | (170%) | 2262.4 | (159%) |
| line 6 | | | | | | | | | | | | |
| 35S:Myb76, | 189.0 | (115%) | 16.1 | (113%) | 0.4 | (46%) | 222.5 | (117%) | 1648.9 | (136%) | 1920.4 | (135%) |
| line 2 | | | | | | | | | | | | |
| 35S:Myb76, | 94.1 | (57%) | 13.8 | (97%) | 0.2 | (30%) | 118.5 | (62%) | 2949.9 | (244%) | 3076.9 | (217%) |
| line 4 | | | | | | | | | | | | |
| 35S:Myb76, | 124.9 | (76%) | 22.7 | (159%) | 0.8 | (101%) | 155.1 | (81%) | 2358.6 | (195%) | 2523.1 | (178%) |
| line 6 | | | | | | | | | | | | |
| 35S:Myb76, | 123.1 | (75%) | 23.7 | (166%) | 0.3 | (36%) | 149.3 | (78%) | 1930.3 | (160%) | 2096.7 | (148%) |
| line 7 | | | | | | | | | | | | |
| 35Senh-Myb76, | 159.0 | (97%) | 12.1 | (85%) | 0.1 | (12%) | 183.5 | (96%) | 1883.5 | (156%) | 2092.2 | (147%) |
| line 4 | | | | | | | | | | | | |
| 35Senh-Myb76, | 130.5 | (80%) | 18.7 | (131%) | 0.4 | (55%) | 151.0 | (79%) | 1650.9 | (137%) | 1809.0 | (127%) |
| line 6 | | | | | | | | | | | | |
| 35Senh.Myb76, | 122.8 | (75%) | 24.2 | (170%) | 1.1 | (138%) | 149.1 | (78%) | 1786.3 | (148%) | 1938.4 | (137%) |
| line 8 | | | | | | | | | | | | |
| Wildtype, Col0 | 163.8 | (100%) | 14.3 | (100%) | 0.8 | (100%) | 190.6 | (100%) | 1209.1 | (100%) | 1419.5 | (100%) |

The increase in GSLs did not confer a visual phenotype to the plants as they all, both in the T2 and the T3 generation, resembled wildtype in appearance.

### Example 10

### Analysis of 35Senh-Myb28 Transformants

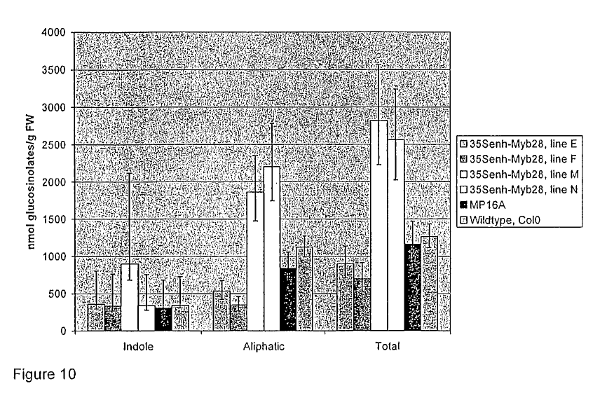

FIG. 10 shows the effect of endogenous overexpression of Myb28 on indole and aliphatic GSL levels in Arabidopsis transgenic lines. The results are listed in Table 3.

Table 3 shows individual GSLs in selected 35Senh-Myb28 lines, the empty vector control (MP16A) and wildtype. Quantities of GSLs are in nmol/g FW and are the mean of the extraction from 4 individual plants (transgenic lines) and 14 (wildtype). The relative change in level of GSL in comparison to wildtype is stated in parenthesis. The abbreviations can be found in the abbreviation list.

From the present example and previous example it can be seen that in addition to affecting total content of aliphatic glucosinolates, the three MYB genes altered the composition of the aliphatic glucosinolates present in the leaves. All three 35S:MYB transgenes resulted in significantly elevated levels of the short-chained 4MSB and 5MSP whereas the level of 4MTB was significantly lowered. In contrast, the different 35S:MYB transgenes exhibited divergent effects on long-chained aliphatic glucosinolates, e.g. methylsulfinyloctyl glucosinolate, 8MSO. Over-expression of MYB29 and MYB76 conferred a significant increase in 8MSO levels, whereas over-expression of MYB28 did not alter the content of 8MSO.

TABLE 3

| | 3MSP | | 4MSB | | 4MTB | | 5MSP | | 5MTP | |
|---|---|---|---|---|---|---|---|---|---|---|
| 35Senh- | 50.7 | (45%) | 348.15 | (51%) | 52.59 | (35%) | 16.04 | (40%) | 1.94 | (14%) |
| Myb28, | | | | | | | | | | |
| line E | | | | | | | | | | |
| 35Senh- | 39.86 | (36%) | 241.09 | (36%) | 20.09 | (13%) | 13.51 | (34%) | 1.28 | (9%) |
| Myb28, | | | | | | | | | | |
| line F | | | | | | | | | | |
| 35Senh- | 138.73 | (124%) | 1263.33 | (186%) | 192.5 | (126%) | 61.4 | (153%) | 11.97 | (88%) |
| Myb28, | | | | | | | | | | |
| line M | | | | | | | | | | |
| 35Senh- | 151.17 | (135%) | 1625.41 | (240%) | 50.15 | (33%) | 93.37 | (232%) | 12.97 | (96%) |
| Myb28, | | | | | | | | | | |
| line N | | | | | | | | | | |
| MP16A | 81.02 | (72%) | 491.87 | (73%) | 113.41 | (75%) | 30.07 | (75%) | 7.76 | (57%) |
| Wildtype, | 112.12 | (100%) | 677.8 | (100%) | 152.18 | (100%) | 40.17 | (100%) | 13.53 | (100%) |
| Col0 | | | | | | | | | | |

| | 7MSH | | 7MTH | | 8MSO | | 8MTO | |
|---|---|---|---|---|---|---|---|---|
| 35Senh- | 8.5 | (60%) | 2.34 | (15%) | 34.61 | (63%) | 13.89 | (48%) |
| Myb28, | | | | | | | | |
| line E | | | | | | | | |
| 35Senh- | 7.51 | (53%) | 1.07 | (7%) | 24.24 | (44%) | 15.82 | (54%) |
| Myb28, | | | | | | | | |
| line F | | | | | | | | |
| 35Senh- | 14.69 | (104%) | 15.85 | (103%) | 84 | (154%) | 60.65 | (209%) |
| Myb28, | | | | | | | | |
| line M | | | | | | | | |
| 35Senh- | 38.26 | (270%) | 10.99 | (71%) | 185.63 | (340%) | 37.7 | (130%) |
| Myb28, | | | | | | | | |
| line N | | | | | | | | |
| MP16A | 11.08 | (78%) | 17.9 | (116%) | 31.2 | (57%) | 39.21 | (135%) |
| Wildtype, | 14.17 | (100%) | 15.43 | (100%) | 54.67 | (100%) | 29.05 | (100%) |
| Col0 | | | | | | | | |

| | i3m | | 4MeOHI3M | | NMeOHI3M | | Indole | | Aliphatic | | Total | |
|---|---|---|---|---|---|---|---|---|---|---|---|---|
| 35Senh- | 320.07 | (111%) | 23.3 | (61%) | 14.57 | (110%) | 358.73 | (105%) | 534.14 | (48%) | 899.99 | (71%) |
| Myb28, | | | | | | | | | | | | |
| line E | | | | | | | | | | | | |
| 35Senh- | 299.88 | (104%) | 18.95 | (50%) | 16.11 | (122%) | 335.18 | (98%) | 349.88 | (31%) | 698.49 | (55%) |
| Myb28, | | | | | | | | | | | | |
| line F | | | | | | | | | | | | |
| 35Senh- | 756.63 | (261%) | 43.71 | (115%) | 91.99 | (697%) | 899.04 | (262%) | 1860.78 | (166%) | 2816.67 | (223%) |
| Myb28, | | | | | | | | | | | | |
| line M | | | | | | | | | | | | |
| 35Senh- | 278.23 | (96%) | 21.98 | (58%) | 33.85 | (257%) | 337.99 | (99%) | 2202.94 | (196%) | 2560.11 | (202%) |
| Myb28, | | | | | | | | | | | | |
| line N | | | | | | | | | | | | |
| MP16A | 246.09 | (85%) | 25.96 | (68%) | 33.07 | (251%) | 306.41 | (89%) | 838.17 | (75%) | 1164.59 | (92%) |
| Wildtype, | 289.49 | (100%) | 38 | (100%) | 13.19 | (100%) | 342.55 | (100%) | 1123.83 | (100%) | 1265.19 | (100%) |
| Col0 | | | | | | | | | | | | |

### Example 11

### Activity of Various FMOs of the Invention

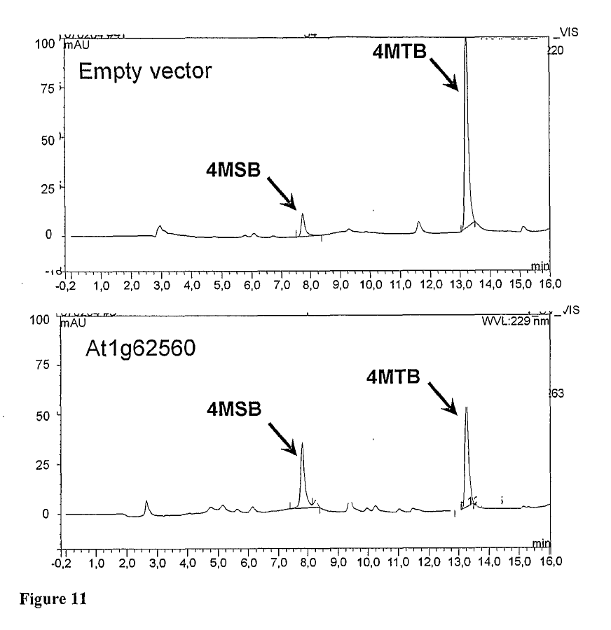

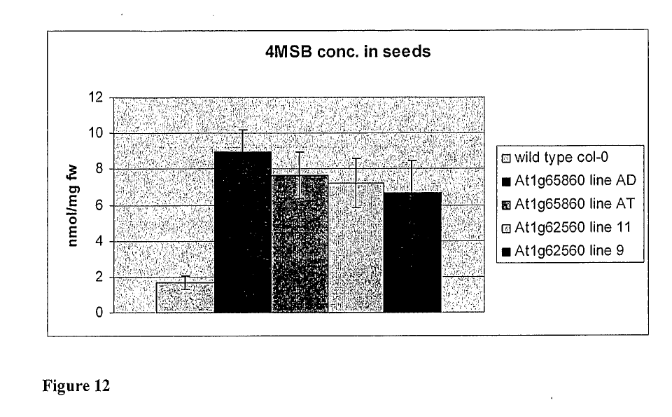

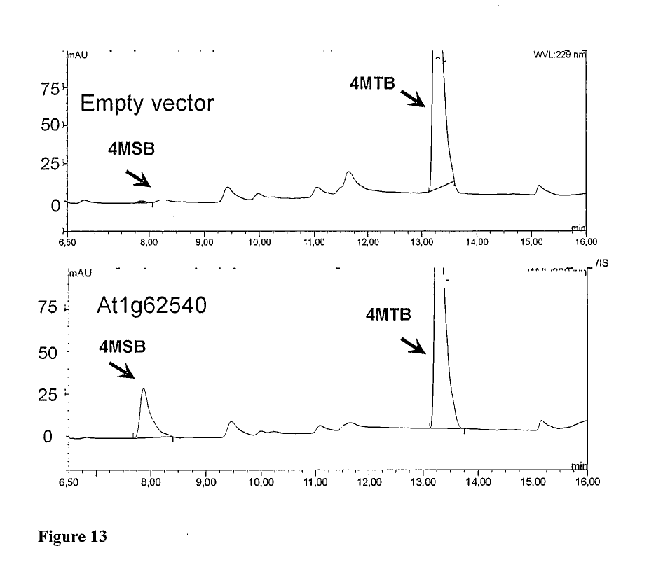

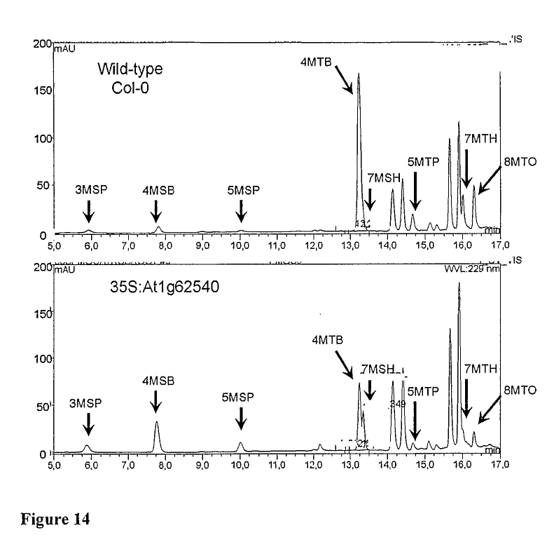

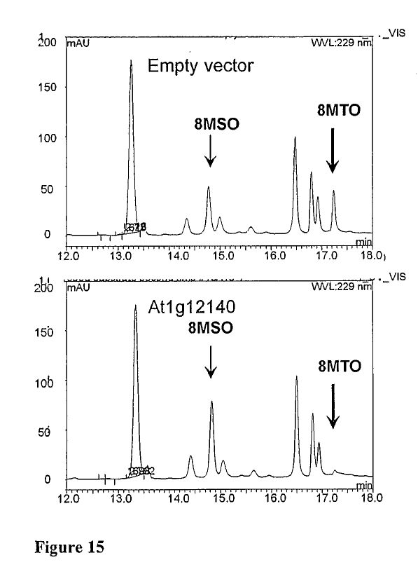

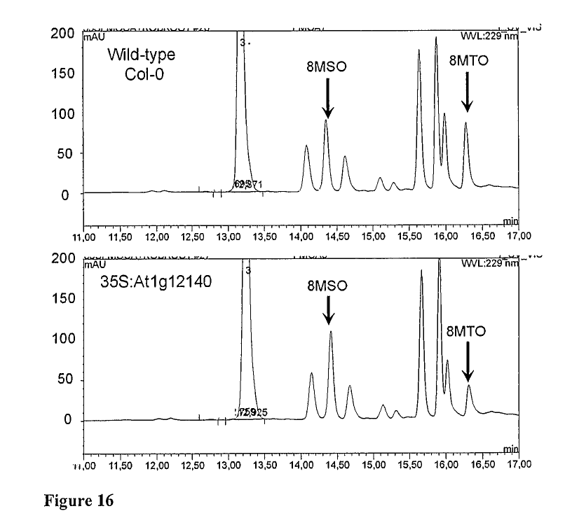

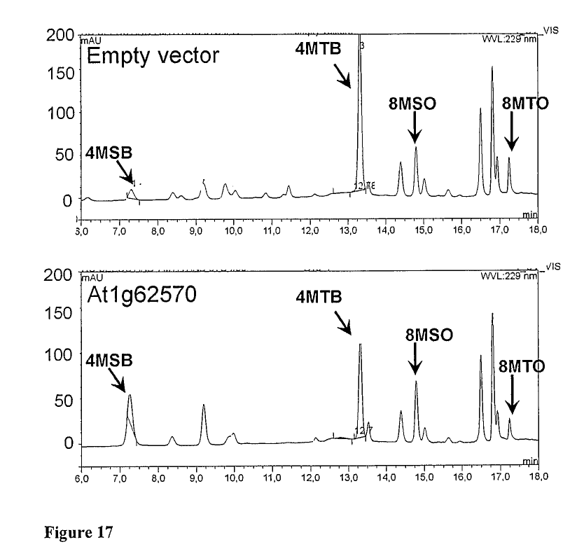

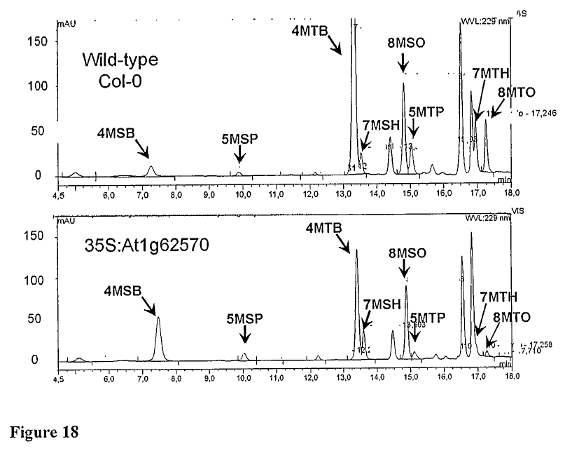

FIGS. 11-18 show the activity of At1g62560, At1g65860, At1g62540, At1g12140 and At1g62570 in E. coli or in seeds using a variety of substrates. It can thus be seen that all 5 of the encoded FMO enzymes have S-oxygenation activity i.e. convert both desulfo and intact methylalkyl glucosinolates into sulfinylalkyl glucosinolates. It can further be seen that At1g65860, At1g62570, At1g62560 and At1g62540 have a broad specificity towards all methylthio (MT) glucosinolates in Arabidopsis, whereas At1g12140 mainly converts long-chain (especially octyl)methylthioalkyl-into methylsulfinyl glucosinolates.

### Example 12

### Accumulation of Aliphatic Glucosinolates in 35S:MYB Over-Expression Lines in Different Tissues

Glucosinolate contents and profiles vary between different tissues (Brown et al., 2003; Petersen et al., 2002). To investigate whether over-expression of the three MYB transcription factors conferred changes to the levels and composition of aliphatic glucosinolates in seeds as well, glucosinolates were extracted and analyzed from seeds from the same plants used glucosinolates in rosette leaves. All 35S:MYB28, 35S:MYB29 and 35S:MYB76 lines showed elevated levels of aliphatic glucosinolates in seeds (Table 4). Similar to what was observed in foliar tissue, the increase of aliphatic glucosinolates in seeds within the 35S:MYB28 lines was entirely due to a rise in short-chained aliphatic glucosinolates. In fact, a significant decrease in long-chained aliphatic glucosinolates was observed (Table 4). Seeds of 35S:MYB29 and 35S:MYB76 lines had an overall increase in total aliphatic glucosinolates with the most significant effects being on the longer of the short-chained glucosinolates, 5MSP, 6MTH and 6MSH (Table 4). Thus, all three MYB genes can specifically alter accumulation of aliphatic glucosinolates when over-expressed using a 35S promoter, and the compositional differences between the 35S:MYB transgenes suggest that they are not operating via identical mechanisms.

TABLE 4
Average Seed Glucosinolate Content in transgenic 35S: MYB lines.

| | Col-0 | | MYB28 | | | MYB29 | | | MYB76 | | |
|---|---|---|---|---|---|---|---|---|---|---|---|
| | N = 8 | | N = 11 | | | N = 11 | | | N = 13 | | |
| Trait | Mean | SE | Mean | SE | Sig | Mean | SE | Sig | Mean | SE | Sig |
| 3MTP | 0.11 | 0.01 | 0.07 | 0.01 | ** | 0.20 | 0.03 | | 0.14 | 0.01 | |
| 3MSP | 0.12 | 0.02 | 0.46 | 0.09 | ** | 0.29 | 0.08 | | 0.17 | 0.03 | |
| 3OHP | 1.10 | 0.08 | 0.87 | 0.11 | | 0.99 | 0.02 | | 1.11 | 0.07 | |
| 3BZOP | 1.56 | 0.09 | 1.59 | 0.11 | | 1.83 | 0.05 | | 1.75 | 0.06 | |
| 4MTB | 13.63 | 1.01 | 18.91 | 1.13 | * | 17.51 | 1.07 | | 16.87 | 0.63 | |
| 4MSB | 1.44 | 0.17 | 6.26 | 1.61 | ** | 2.71 | 0.64 | | 2.15 | 0.29 | |
| 4OHB | 1.88 | 0.17 | 1.45 | 0.13 | | 1.57 | 0.11 | | 2.17 | 0.14 | |
| 4BZOB | 1.97 | 0.06 | 1.64 | 0.18 | | 1.85 | 0.14 | | 2.24 | 0.08 | * |
| 5MSP | 0.10 | 0.01 | 0.71 | 0.20 | ** | 0.26 | 0.06 | ** | 0.18 | 0.01 | ** |
| 6MTH | 0.06 | 0.01 | 0.14 | 0.02 | ** | 0.11 | 0.02 | * | 0.12 | 0.01 | ** |
| 6MSH | 0.07 | 0.00 | 0.18 | 0.08 | | 0.13 | 0.02 | * | 0.10 | 0.01 | ** |
| 7MTH | 1.34 | 0.07 | 0.63 | 0.19 | ** | 1.46 | 0.05 | | 1.45 | 0.09 | |
| 7MSO | 0.52 | 0.02 | 0.55 | 0.20 | | 0.71 | 0.06 | * | 0.64 | 0.04 | * |
| 8MTO | 2.25 | 0.14 | 0.42 | 0.15 | ** | 2.03 | 0.11 | | 2.14 | 0.14 | |
| 8MSO | 4.06 | 0.17 | 2.44 | 0.67 | * | 4.60 | 0.13 | | 4.73 | 0.29 | |
| Total | 30.97 | 1.57 | 37.99 | 3.41 | * | 37.42 | 1.85 | * | 37.01 | 1.20 | * |
| Aliphatic | | | | | | | | | | | |
| I3M | 0.80 | 0.06 | 0.75 | 0.09 | | 0.66 | 0.06 | * | 0.72 | 0.05 | |
| 4OH-I3M | 0.01 | 0.00 | 0.03 | 0.00 | ** | 0.01 | 0.00 | | 0.01 | 0.00 | |
| Total Indole | 1.05 | 0.06 | 0.95 | 0.11 | | 0.86 | 0.07 | * | 0.99 | 0.05 | |
| Isoleucine | 0.74 | 0.08 | 1.53 | 0.23 | ** | 1.11 | 0.13 | | 0.99 | 0.10 | |

Mean shows the average glucosinolate content in nmol per mg of tissue. SE is the standard error of the mean for that line. This data represents using eight plants for Col-0, 11 plants containing the 35S:MYB28 transgene (five and six plants from two independent transgenic lines), eleven plants containing the 35S:MYB76 transgene (five and six plants each from two independent transgenic lines) and thirteen plants containing the 35S:MYB29 transgene (six and seven plants from two independent transgenic lines). Sig indicates the P value of the difference between Col-0 and transgenic lines containing the respective 35S:MYB transgene as determined by ANOVA. One asterisk represents a P value between 0.05 and 0.005 while two asterisks is a P value below 0.005. Cells with no asterisk represent non-significant P values, those greater than 0.05. N represents the total number of independent samples per genotypic class.

### Example 13

### Gene Expression Analysis in 35S:MYB Over-Expression Lines

The observation that over-expression of the three MYB genes resulted in elevated content of aliphatic glucosinolates led us to test if the transcript levels of genes in the biosynthetic pathway were concurrently elevated in the different genotypes. Affymetrix ATH1 genechip microarrays were used to measure transcript accumulation in wild-type and the two selected transgenic lines for each 35S:MYB transgene. As for the glucosinolate analysis described above, an ANOVA analysis of transcript levels between the selected transgenic lines showed no significant difference, which allowed us to combine the mean of the two independent lines to test the effect of the introducing the MYB transgene rather than the effect of one single transgenic line versus wild-type.

Elevated accumulation of aliphatic glucosinolates might be expected to affect the entire sulphur metabolism of the plant due to the pull on the methionine pool. Consequently, we utilized a pathway ANOVA (Kliebenstein et al., 2006b) approach to test the impact of the MYB over-expression on the major sulfur-utilization pathways, i.e. sulfate assimilation, cysteine production, methionine production, aliphatic glucosinolates, indole glucosinolates, homocysteine conversion and SAM production, as well as on the characterized transcription factors for indole and aliphatic glucosinolates.

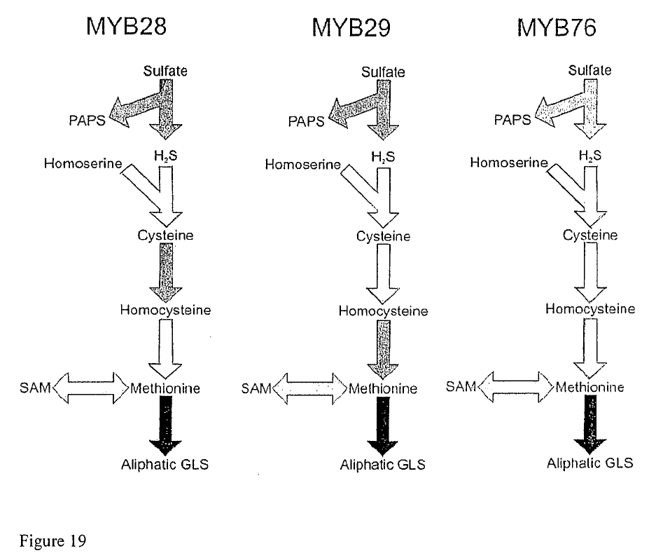

The pathway ANOVA revealed that the primary effect of the three 35S:MYB transgenes within these pathways is to induce the biosynthesis of aliphatic glucosinolates since this was the pathway showing the largest effect in both magnitude and statistical support (FIG. 19). Another common effect of over-expression of the three MYB genes was to lower transcript levels for genes required to convert methionine into SAM. This could potentially increase the pool of methionine available for production of aliphatic glucosinolates (FIG. 19). In addition, all three MYB genes altered transcript level for genes in the biosynthesis of PAPS (3′-phosphoadenosyl-5′-phosphosulfate) which is the substrate required for the sulfotransferases catalyzing the final step of glucosinolate core synthesis. Interestingly, MYB28 and MYB29 induced the genes required for PAPS production whereas MYB76 appeared to repress their transcript levels.

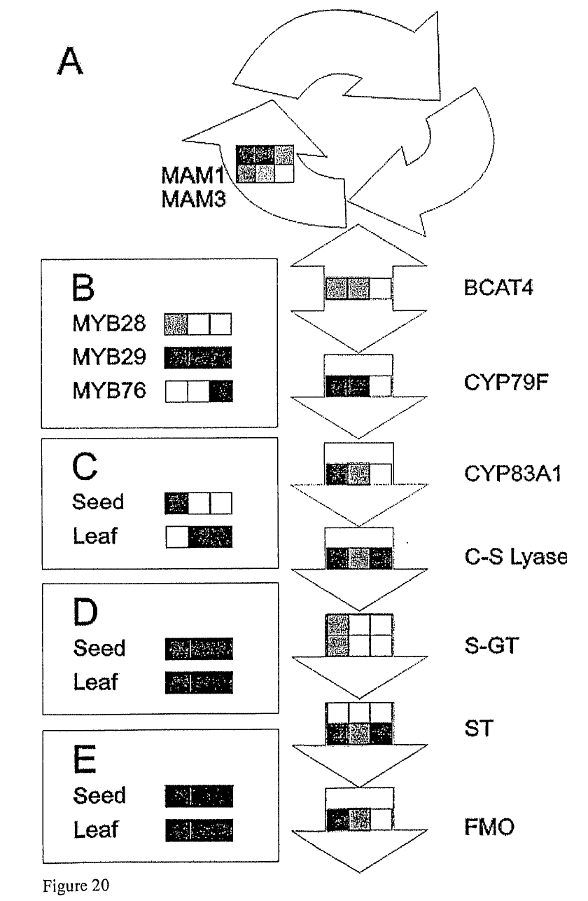

Within the pathway ANOVAs, we next utilized F-tests to test if the transcripts for the individual genes in the biosynthesis of aliphatic glucosinolates were altered in comparison to wild-type Col-0. As expected, the individual 35S:MYB lines led to elevated accumulation of the specific MYB gene that was over-expressed (FIG. 20). However, even though the transcript levels of MYB28 was significantly elevated, its modest increase of approximately 40% was in contrast to the more dramatic elevation of approximately 200 and 500% in transcript level of MYB29 and MYB76 within the 35S:MYB29 and 35S:MYB76 lines, respectively. The modest increase in MYB28 expression is, however, sufficient to result in an increased accumulation of glucosinolates in the 35S:MYB28 lines (see e.g. Table 4). Further, MYB29 transcript accumulated in response to over-expressing MYB28 and MYB76 (FIG. 20) suggesting the presence of some interplay between the MYB genes.

Over-expression of MYB28 and MYB29 led to statistically significant increases in transcript levels for, respectively, eleven and nine of the genes experimentally demonstrated to be involved in aliphatic glucosinolate biosynthesis or regulation. Additionally, the 35S:MYB28 and 35S:MYB29 lines induced, respectively, six and four genes of the seven genes proposed to be involved in biosynthesis of aliphatic glucosinolates. In contrast to the other aliphatic biosynthetic genes that are induced, MAM3 had lower transcript levels in 35S:MYB28 and 35S:MYB29 lines in comparison to Col-0 with the lowest level in 35S:MYB28 lines (FIG. 20). The 35S:MYB76 lines upregulated relatively fewer transcripts of aliphatic biosynthetic genes as it showed altered transcript levels for only six of the characterized and four of the proposed aliphatic biosynthetic genes (FIG. 20). The ANOVA results obtained from pathways as well as the individual genes in aliphatic glucosinolate biosynthesis suggest that in addition to having significant functional overlap, MYB28, MYB29 and MYB76 differ in their regulatory capacities or targets.

### Example 14

### Genome-Wide Transcript Effects of 35S:MYB Over-Expression Lines

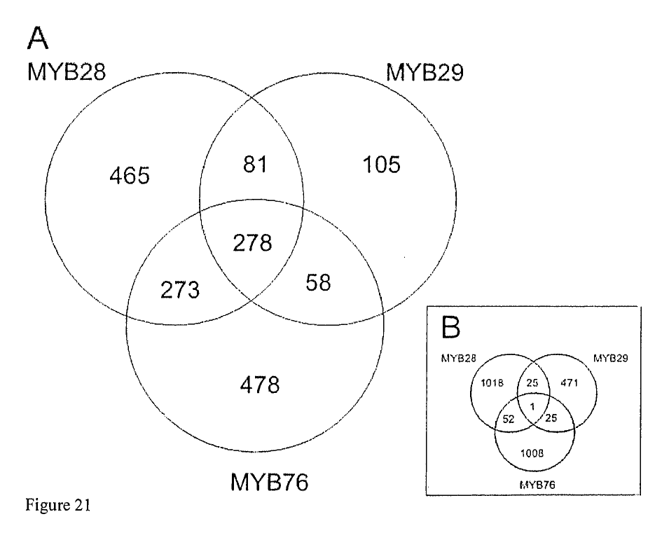

To better assess the overlap of transcripts altered in 35S:MYB lines, we conducted separate ANOVAs to test every transcript for a significant difference between wild-type and each selected 35S:MYB line. Using a false discovery rate (FDR) of 0.05, data indicated that the 35S:MYB28 lines altered the accumulation of 1097 transcripts, the 35S:MYB29 lines 522 transcripts and the 35S:MYB76 lines 1087 transcripts (FIG. 21). The effects were nearly equally divided between transcripts induced and those repressed. In agreement with the hypothesis that all three MYB genes share regulatory targets such as aliphatic glucosinolates, there was a significant bias for overlap in transcripts regulated by all three MYB genes (P<0.001 by Chi-square, FIG. 21). This overlap included approximately 53% of all transcripts regulated by MYB29. In contrast to the overlap, there are a number of genes altered by specific subsets of the MYBs further suggesting that they are not operating via a single mechanism (FIG. 21). Decreasing the FDR rate to 0.10 did not alter the ratio of transcripts populating the regions of the Venn diagram suggesting that the indication of specificity amongst the factors is not merely a statistical artifact of the microarray analysis (data not shown). An analysis of GO annotations in all quadrants of the Venn diagram did not show any informative bias with regards to function of the genes whose transcripts were affected (data not shown).

Previous work has suggested a link between aliphatic glucosinolates and the accumulation of sinapate esters whereby the glucosinolate biosynthetic mutants altered sinapate accumulation and vice-versa (Hemm et al., 2003; Kliebenstein et al., 2005). Interestingly, all three 35S:MYB genotypes led to decreased transcript levels of MYB4-At4g38620, a MYB transcription factor which suppresses the accumulation of sinapate esters (Jin et al., 2000). Accordingly, transcript levels of SNG1-At2g22990, the gene responsible for conversion of sinapoyl glucose to sinapoyl malate (Lehfeldt et al., 2000) are increased in all three genotypes. Curiously, however, BRT1-At3g21560, responsible for the conversion of sinapate to sinapoyl glucose (Sinlapadech et al., 2007), on the other hand, are down-regulated in all three. This suggests that the MYBs may be involved in the suggested cross-talk between sinapate and aliphatic glucosinolate metabolism.

### Example 15

### Genome-Wide Transcript Effects of 35S:MYB Over-Expression Lines myb28-1, myb29-1 and -2 and myb76-1 and -2 Knockout Mutants Display Reduction in Different Aliphatic Glucosinolates

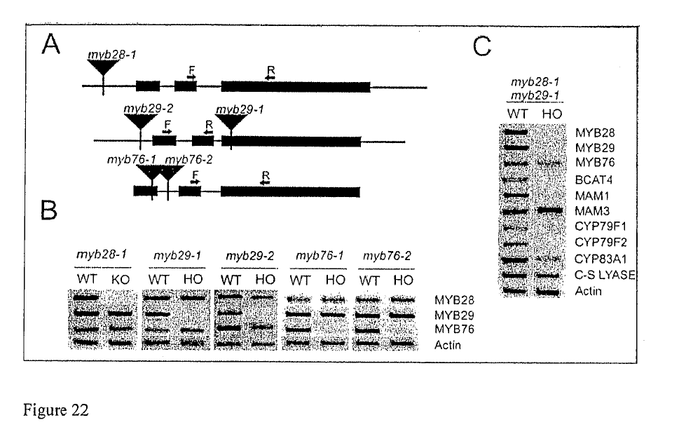

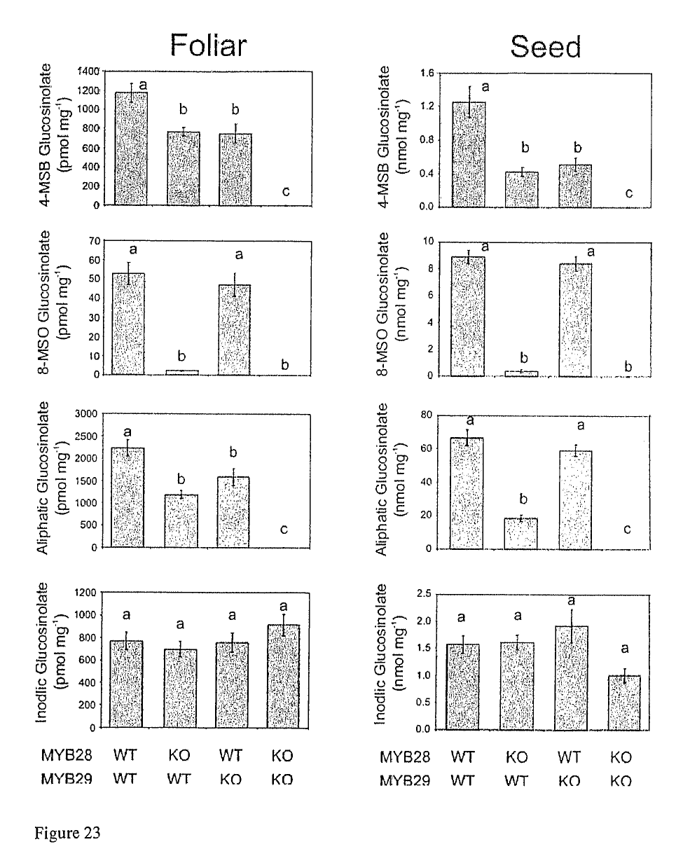

To further validate that the MYB candidate genes play a role in biosynthesis of aliphatic glucosinolates in planta, loss-of-function alleles in MYB28, MYB29 and MYB76 were obtained (Alonso et al., 2003; Rosso et al., 2003; Tissier et al., 1999) and the borders of the T-DNA insertions sequenced to validate the insertion site (FIG. 22A). The transcript levels for the MYB genes were measured in all five lines, myb28-1, myb29-1, myb29-2, myb76-1 and myb76-2, to determine whether the T-DNA insertions resulted in a loss of transcript. RT-PCR was conducted on RNA purified from at least two independent wild-type plants and homozygous single-mutant plants and with at least two different cycle numbers to better quantify changes. The analysis revealed that myb28-1, myb29-1 and myb29-2 were indeed knockout mutants whereas myb76-1 and myb76-2 were knockdown mutants as they still had residual but much reduced transcript levels (FIG. 22B). The knockout or knockdown of one MYB transcription factor did not lead to changes in levels of any of the other MYB transcription factors. No apparent visual phenotype was observed in any of the single knockout mutants under the conditions tested. The impact on the transcript level for the individual biosynthetic genes in the knockouts was minimal with only a slight reduction in BCAT4, MAM1 and CYP79F1 transcripts in myb29-1 and myb29-2 and in MAM1, CYP79F2 and CYP83A1 transcripts in myb28-1 (data not shown). It is possible that levels of aliphatic glucosinolates are sensitive to changes in aliphatic glucosinolate transcript accumulation that are not easily detectable using quantitative RT-PCR. This could be enhanced as the level of a transcripts is a single time-point measure while the level of aliphatic glucosinolates likely integrates over a length of time, thereby amplifying any change.

To minimize maternal effects and to show that any observed chemotype segregated with the T-DNA insertion, we genotyped germinating offspring from segregating heterozygous mutants and measured glucosinolates on the homozygous knockout and wild-type sibling progeny for each mutant. The combined mean from myb29-1 and myb29-2 and that of myb76-1 and myb76-2 are presented since ANOVA showed that there was no difference in the relative levels of glucosinolates between the different mutant alleles. Leaves from the myb29-1, myb29-2, myb76-1 and myb76-2 mutants had significantly reduced levels of short-chained aliphatic glucosinolates content with no change in the amounts of the long-chained aliphatic glucosinolates (Table 5). In contrast, the myb28-1 mutant showed a dramatic reduction in long-chained and a decrease in short-chained aliphatic glucosinolates (Table 5). These results indicate that MYB29 and MYB76 play a role for regulation of short-chained aliphatic glucosinolates, whereas MYB28 plays a role in the control of both short- and long-chained aliphatic glucosinolates in leaves. The mutations in MYB28, MYB29 and MYB76 did not affect indole glucosinolate levels (Table 5).

In Table 5, plants are derived from progeny of mutants heterozygous for the myb28-1, myb29-1, myb29-2, myb76-1 and myb76-2 allele. Mean shows the average glucosinolate content in pmol per mg of fresh weight tissue. SE is the standard error of the mean for that line. This data represents two independent biological replicates, except for myb76 which only has one replicate. The data for the two myb29 alleles and the two myb76 alleles were pooled as there was no significant difference in the glucosinolate phenotype between the different alleles. Sig indicates the P value of the difference between Col-0 wild-type and the transgenic lines as determined by ANOVA. One asterisk represents a P value between 0.05 and 0.005 while two asterisks is a P value below 0.005. Cells with no asterisk represent non-significant P values, those greater than 0.05. N represents the total number of independent samples per genotypic class.

TABLE 5
Foliar glucosinolate content in T-DNA insertion mutants of MYB28, MYB29 and MYB76.

| | myb28-1 | | | | | myb29 | | | | | myb76 | | | | |
|---|---|---|---|---|---|---|---|---|---|---|---|---|---|---|---|
| | WT | | Homozygous | | | WT | | Homozygous | | | WT | | Homozygous | | |
| | N = 6 | | N = 12 | | | N = 24 | | N = 16 | | | N = 6 | | N = 10 | | |
| | Mean | SE | Mean | SE | Sig | Mean | SE | Mean | SE | Sig | Mean | SE | Mean | SE | Sig |
| 4MSB | 140 | 37 | 65 | 20 | | 90 | 9 | 69 | 0.1 | ** | 60 | 6 | 40 | 4 | * |
| 4MTB | 49 | 14 | 24 | 9 | | 24 | 5 | 21 | 0.1 | ** | 7 | 1 | 3 | 1 | ** |
| 5MSP | 11 | 2 | 7 | 2 | | 8 | 1 | 5 | 0 | ** | 7 | 1 | 5 | 0 | |
| 7MSH | 8 | 2 | 2 | 1 | ** | 6 | 0 | 8 | 0 | | 5 | 0 | 5 | 0 | |
| 7MTH | 5 | 2 | 1 | 0 | ** | 3 | 1 | 5 | 0 | | 1 | 0 | 1 | 0 | |
| 8MSO | 38 | 11 | 5 | 1 | ** | 26 | 2 | 37 | 0.1 | | 20 | 2 | 20 | 2 | |
| 8MTO | 1 | 0 | 0 | 0 | ** | 2 | 0 | 3 | 0 | | 1 | 0 | 2 | 0 | |
| Total | 250 | 65 | 111 | 33 | | 159 | 16 | 147 | 0.2 | | 100 | 8 | 76 | 8 | * |
| aliphatic | | | | | | | | | | | | | | | |
| I3M | 77 | 13 | 82 | 18 | | 70 | 7 | 97 | 0.2 | | 42 | 4 | 51 | 3 | |
| 4MO- | 4 | 1 | 5 | 1 | | 5 | 0 | 5 | 0 | | 5 | 0 | 5 | 0 | |
| I3M | | | | | | | | | | | | | | | |
| NMO- | 7 | 2 | 9 | 6 | | 5 | 1 | 9 | 0 | | 1 | 0 | 2 | 0 | |
| I3M | | | | | | | | | | | | | | | |
| Total | 88 | 15 | 96 | 24 | | 80 | 8 | 112 | 0.2 | | 49 | 4 | 59 | 4 | |
| indole | | | | | | | | | | | | | | | |

The impact of the myb28-1 or myb29-1 insertions on the glucosinolate pool in seeds was measured on seeds derived from the plants on which glucosinolates in leaves had been measured. This showed that the levels of long-chained aliphatic glucosinolates were significantly reduced in homozygous myb28-1 seeds (Table 6). Furthermore, a decrease was observed in short-chained aliphatic glucosinolates which along with the decrease in long-chained led to a substantial reduction in total amounts of aliphatic glucosinolates (Table 6). For myb29-1, a significant reduction was observed in the levels of the short-chained 4MTB and 4MSB, with no impact on long-chained aliphatic glucosinolates (Table 6). This further supports the observation that both MYB28 and MYB29 play a role in controlling the accumulation of aliphatic glucosinolates in leaves and seeds, but via different chain-length specificities. The level of indole glucosinolates was not affected in either line.

TABLE 6
Seed glucosinolate content in the myb28-1 and myb29-1mutants.

| | myb28-1 | | | | | myb29-1 | | | | |
|---|---|---|---|---|---|---|---|---|---|---|
| | WT | | Homozygous | | | WT | | homozygous | | |
| | N = 10 | | N = 9 | | | N = 8 | | N = 11 | | |
| | Mean | SE | Mean | SE | Sig | Mean | SE | Mean | SE | Sig |
| 3BZOP | 3.32 | 0.34 | 2.63 | 0.37 | | 3.60 | 0.31 | 3.05 | 0.18 | |
| 4MSB | 1.82 | 0.48 | 1.39 | 0.79 | | 1.69 | 0.47 | 1.26 | 0.33 | * |
| 4MTB | 31.14 | 3.19 | 21.79 | 3.64 | | 33.64 | 3.24 | 24.82 | 1.11 | * |
| 4BZOB | 4.34 | 0.47 | 6.51 | 0.88 | * | 4.55 | 0.42 | 5.64 | 0.68 | ** |
| 5MTP | 1.98 | 0.24 | 1.27 | 0.22 | * | 2.16 | 0.23 | 1.58 | 0.15 | |
| 7MSH | 1.18 | 0.22 | 0.01 | 0.01 | ** | 1.43 | 0.28 | 1.65 | 0.32 | |
| 7MTH | 3.03 | 0.50 | 0.94 | 0.72 | * | 3.54 | 0.47 | 3.36 | 0.46 | |
| 8MSO | 7.74 | 0.60 | 0.44 | 0.15 | ** | 8.31 | 0.68 | 7.59 | 0.46 | |
| 8MTO | 5.06 | 0.76 | 3.74 | 3.26 | | 5.67 | 0.76 | 5.67 | 0.96 | |
| Total | 59.61 | 5.39 | 40.72 | 5.61 | * | 64.58 | 5.45 | 54.63 | 2.98 | |
| aliphatic | | | | | | | | | | |
| I3M | 1.86 | 0.37 | 2.13 | 0.36 | | 2.21 | 0.34 | 1.73 | 0.31 | |

Seeds are derived from homozygous or wild-type progeny of a mutant heterozygous for either the myb29-1 or the myb28-1 allele. Mean shows the average glucosinolate content in nmol/10 seeds. SE is the standard error of the mean for that line. Sig indicates the P value of the difference between Col-0 wild-type and the homozygous mutant lines as determined by ANOVA. One asterisk represents a P value between 0.05 and 0.005 while two asterisks is a value below 0.005. Cells with no asterisk represent non-significant P values, those greater than 0.05. N represents the total number of independent samples per genotypic class.

### Example 16

### A myb28-1 myb29-1 Double Knockout Mutant Displays No Detectable Aliphatic Glucosinolates and Reduced Transcript Level of Biosynthetic Enzymes

To further investigate the roles of MYB28 and MYB29 on production of aliphatic glucosinolates, we crossed myb28-1 and myb29-1 to obtain a double knockout mutant. Homozygous double knockouts were obtained. Analysis of transcript accumulation within the homozygous double knockout in comparison to the WT Col-0 showed that transcripts of BCAT4, MAM1, CYP79F1 and CYP79F2 were undetectable in the double knockout leaves under the cycle numbers tested. Furthermore, a substantial reduction was observed in CYP83A1 and C-S-LYASE transcripts (FIG. 22C). Finally, the absence of MYB28 and MYB29 transcripts resulted in a small decrease in the transcript level of MYB76 (FIG. 22C). We were unable to detect aliphatic glucosinolates in either the leaves or seeds of the homozygous double myb28-1 myb29-1 mutant (FIG. 23). In comparison to the WT and homozygous single knockouts, this loss of glucosinolates showed a statistically significant epistatic interaction between MYB28 and MYB29. A merely additive interaction would have led to foliar total aliphatic glucosinolates having a level of 25% of wildtype in the double mutants (FIG. 23). The differences in MYB29 impacts on total aliphatic glucosinolates between the two tissues is likely due to the increased proportion of long chain aliphatic glucosinolates in the seed in comparison to the leaf. This data confirm that in addition to having specific activities, MYB28 and MYB29 also have synergistic functionalities. Indole glucosinolate levels were not affected in the myb28-1 myb29-1 double knockout mutant in comparison to the WT or homozygous single knockout lines. The loss of aliphatic glucosinolates in the double knockout plants could not have been predicted by the chemotype of the single knockout mutants and as such reveals an emergent property of glucosinolate regulation. Additionally, this double knockout phenotype suggests that MYB76 requires a functional MYB28 and MYB29 to control aliphatic glucosinolates.

### Discussion of Examples 7 to 16

When the three MYB genes described above were individually over-expressed in a wildtype Col-0 background, all lines accumulated more aliphatic glucosinolates in leaves and seeds. Microarray analysis showed that transcript levels for genes involved in biosynthesis of aliphatic glucosinolates in foliar tissues were concurrently up-regulated. This showed that all three MYB genes have the may up-regulate the accumulation of aliphatic glucosinolates via increasing the biosynthetic transcripts. Analysis of knockout mutants of MYB28, MYB29 and MYB76 further established a role of the three MYB genes in regulation of aliphatic glucosinolates in Col-0 since absent expression of the genes led to reduced contents of aliphatic glucosinolates, as evidenced by altered profiles in both leaves and seeds.

The identification of these three MYBs within a single clade and their overlapping phenotypes in the 35S:MYB over-expressor lines suggested that they may be a redundant gene family. However, analysis of the knockout lines at the metabolic level showed that these functions were not redundant as MYB29 and MYB76 controlled short-chained aliphatic glucosinolates and MYB28 controlled both short- and long-chained aliphatic glucosinolates. The impact of MYB28 on both chain lengths is likely mediated through an impact on CYP79F expression as the MYB281MYB29 double knockout does not impact MAM3 expression. Similar results were obtained with the myb28-1 knockout by Hirai et al. (2007). The individual knockout mutants had only minimal effects on transcripts for the individual genes involved in biosynthesis of aliphatic glucosinolates (data not shown). In contrast, the myb28-1 myb29-1 double knockout mutant dramatically diminished most transcripts for the biosynthetic genes and reduced the contents of all aliphatic glucosinolates. This shows that this family of MYBs functions to regulate the aliphatic glucosinolate biosynthetic pathway in Col-0 and that they have evolved specific and overlapping functions that show complex interconnectivity.

The glucosinolate profiles of the single knockout mutants suggest that MYB28 and MYB29 play significant, but distinct roles in regulation of the biosynthetic genes for aliphatic glucosinolates as both lead to lower levels of specific aliphatic glucosinolates. However, transcript levels were only minimally affected by mutations in the individual genes (data not shown). A myb28-1 myb29-1 double knockout mutant showed that both genes apparently positively interact to control both transcript levels and metabolite accumulation for the majority of the pathway. The total level of aliphatic glucosinolates of the double knockout mutant were dramatically lower than either single knockout mutant in the leaves, and below the level of detection for all aliphatic glucosinolates in both leaves and seeds. In concordance, the transcripts of most characterized aliphatic biosynthetic genes were undetectable in the leaves of the double knockout mutant. None of the phenotypes of the single mutants hinted at the striking phenotype of the double knockout mutant and, as such, the analysis of the latter identifies an emergent property of the glucosinolate regulation system not readily predictable from the phenotypes of the single knockout mutants.

The double knockout analysis points to an interplay between MYB28 and MYB29 whereby they interact to activate the aliphatic glucosinolate pathway. One source of possible MYB interplay was observed in the 35S:MYB lines, where over-expression of MYB28 and MYB76 led to increased levels of MYB29 transcript. This suggests a different role for MYB29, in which it integrates signals from MYB28 and MYB76 in regulating the aliphatic glucosinolates. However, this is not a strictly linear pathway where MYB28 and MYB76 would regulate MYB29 to regulate the glucosinolates since MYB29 transcript seems unchanged in the myb28-1, myb76-1 and myb76-2 mutants. The observation that the myb28-1 myb29-1 double knockout mutant altered transcripts more dramatically than either individual knockout suggests that MYB28 has regulatory functionalities independent of MYB29.

An important factor in production of a given compound is the availability of precursor substrates. The similar level of total aliphatic glucosinolates (although with a different composition) in knockout mutants of either MAM1 and MAM3 (Textor et al., 2007) suggests that under normal conditions in Col-0, a predetermined amount of substrate is destined to go into the glucosinolate pathway. Over-expression of the MYB regulators allows more substrate to enter the glucosinolate pathway as evidenced by the observed increase of up to 110% in total aliphatic glucosinolate content. This is reflected in the altered levels of transcripts for both the biosynthetic as well as for the substrate pathways, although we cannot conclude if the latter is a direct or indirect effect of the MYB over-expression. Compared to the variation in aliphatic glucosinolate content among the Arabidopsis accessions (Kliebenstein et al., 2001b), the increase in aliphatic glucosinolate content modulated by the individual MYB genes can be regarded as rather modest. This suggests a putative restraint, when modifying a single gene, possibly due to a limitation in the substrate availability or in other components of the regulatory machinery. Interestingly, Gigolashvili et al. (2007) describe a line with a seven fold increase in 4MSB when over-expressing MYB28. However, this line shows a strong phenotype which could be due to a strong pull on the methionine pool. This suggests that when the production of aliphatic glucosinolates reaches a certain level due to, for instance, the manipulation of a single regulatory gene, plant growth is hampered by e.g. a shortage of methionine for protein biosynthesis. The alteration of expression levels of multiple genes within the natural accessions may allow for this bottleneck to be bypassed.

### REFERENCES

Kliebenstein, D., J, J. Gershenzon, and T. Mitchell-Olds, 2001a Comparative quantitative trait loci mapping of aliphatic, indolic and benzylic glucosinolate production in Arabidopsis thaliana leaves and seeds. Genetics 159: 359-370.
Stracke, R. M., Werber, and B. Weisshaar, 2001 The R2R3-MYB gene family in Arabidopsis thaliana. Curr. Opin. Plant Biol. 4: 447-456.
Weigel, D., J., H. Ahn, M. A. Blazquez, J. O. Borevitz, S. K. Christensen et al. 2000 Activation tagging in Arabidopsis. Plant Physiol 122: 1003-1013.
Clough, S. J. & Bent, A. F. (1998). Floral dip: a simplified method for Agrobacterium-mediated transformation of Arabidopsis thaliana. Plant J. 16, 735-743.
Dawson, G. W., A. J. Hick, R. N. Bennett, A. Donald, J. A. Pickett, R. M. Wallsgrove, 1993. Synthesis of glucosinolate precursor and investigations into the biosynthesis of phenylalkyl- and methylthioalkylglucosinolates. JBC 268: 27154-27159
Giamoustaris, A. and R. Michen. (1995) The effect of modifying the glucosinolate content of leaves of oilseed rape (Brassica napus ssp. oleifera) on its interaction with specialist and generalist pests. Annals of applied biology 126: 347-363.
Fahey, J. W., X. Haristoy, P. M. Dolan, T. W. Kensler, I. Scholtus, K. K. Stephenson, P. Talalay and A. Lozniewski. (2002) Sulforaphane inhibits extracellular, intracellular, and antibiotic-resistant strains of Helicobacter pylori and prevents benzo[a]pyrene-induced stomach tumors. PNAS 99: 7610-7615.
McNaughton, S. A. and G. C. Marks (2003). Development of a food composition database for the estimation of dietary intakes of glucosinolates, the biologically active constituents of cruciferous vegetables. British Journal Of Nutrition 90 (3): 687-697.
Nitz, G. M. and W. H. Schnitzler (2002). Variation of the glucosinolate content of the rucola species Eruca sativa and Diplotaxis tenuifolia depending on the number of cut. Journal Of Applied Botany-Angewandte Botanik 76 (3-4): 82-86
Nour-Eldin, H. H., B. G. Hansen, M. H. H. Noerholm, J. K. Jensen. and B. A. Halkier. (2006) Advancing uracil-excision based cloning towards an ideal technique for cloning PCR fragments. Nucleic Acids Res. 34:e122.
Schwab, R., S. Ossowski, M. Riester, N. Warthmann. and D. Weigel (2006). Highly specific gene silencing by artificial microRNAs in Arabidopsis. Plant Cell 18: 1121-1133.
Shen, W. J. & Forde, B. G. (1989). Efficient transformation of Agrobacterium spp. by high voltage electroporation. Nucleic Acids Res. 17, 8385.
Zambryski, P., Joos, H., Genetello, C., Leemans, J., Van Montagu, M. and Schell, J. (1983) Ti plasmid vector for the introduction of DNA into plant cells without alteration of their normal regeneration capacity. The Embo Journal, 2, 2143-2150.

### Additional References

Agarwal, M., Hao, Y., Kapoor, A., Dong, C. H., Fujii, H., Zheng, X., and Zhu, J. K. (2006). A R2R3 type MYB transcription factor is involved in the cold regulation of CBF genes and in acquired freezing tolerance. J. Biol. Chem. 281:37636-37645.
Alonso, J. M., Stepanova, A. N., Leisse, T. J., Kim, C. J., Chen, H., Shinn, P., Stevenson, D. K., Zimmerman, J., Barajas, P., Cheuk, R., Gadrinab, C., Heller, C., Jeske, A., Koesema, E., Meyers, C. C., Parker, H., Prednis, L., Ansari, Y., Choy, N., Deen, H., Geralt, M., Hazari, N., Hom, E., Karnes, M., Mulholland, C., Ndubaku, R., Schmidt, I., Guzman, P., guilar-Henonin, L., Schmid, M., Weigel, D., Carter, D. E., Marchand, T., Risseeuw, E., Brogden, D., Zeko, A., Crosby, W. L., Berry, C. C., and Ecker, J. R. (2003). Genome-Wide Insertional Mutagenesis of Arabidopsis thaliana. Science 301:653-657.
Basten, C. J., Weir, B. S., and Zeng, Z.-B. (1999). QTL Cartographer, version 1.13. Department of Statistics, North Carolina State University, Raleigh, N.C.
Bender, J. and Fink, G. R. (1998). A Myb homologue, ATR1, activates tryptophan gene expression in Arabidopsis. Proc. Natl. Acad. Sci. U.S.A 95:5655-5660.
Benjamini, Y. and Hochberg, Y. (1995). Controlling The False Discovery Rate—A Practical and Powerful Approach to Multiple Testing. J. R. Statist. Soc. B 57:289-300.
Bennett, R. N., Wenke, T., Freudenberg, B., Mellon, F. A., and Ludwig-Muller, J. (2005). The tu8 mutation of Arabidopsis thaliana encoding a heterochromatin protein 1 homolog causes defects in the induction of secondary metabolite biosynthesis. Plant Biol. (Stuttg) 7:348-357.
Brader, G., Tas, E., and Palva, E. T. (2001). Jasmonate-dependent induction of indole glucosinolates in Arabidopsis by culture filtrates of the nonspecific pathogen Erwinia carotovora. Plant Physiol 126:849-860.
Brem, R. B., Yvert, G., Clinton, R., and Kruglyak, L. (2002). Genetic dissection of transcriptional regulation in budding yeast. Science 296:752-755.
Brown, P. D., Tokuhisa, J. G., Reichelt, M., and Gershenzon, J. (2003). Variation of glucosinolate accumulation among different organs and developmental stages of Arabidopsis thaliana. Phytochemistry 62:471-481.
Celenza, J. L., Quiel, J. A., Smolen, G. A., Merrikh, H., Silvestro, A. R., Normanly, J., and Bender, J. (2005). The Arabidopsis ATR1 Myb transcription factor controls indolic glucosinolate homeostasis. Plant Physiol 137:253-262.
Churchill, G. A. and Doerge, R. W. (1994). Empirical threshold values for quantitative trait mapping. Genetics 138:963-971.
Doerge, R. W. (2002). Mapping and analysis of quantitative trait loci in experimental populations. Nat. Rev. Genet. 3:43-52.
Doerge, R. W. and Churchill, G. A. (1996). Permutation tests for multiple loci affecting a quantitative character. Genetics 142:285-294.
Field, B., Cardon, G., Traka, M., Botterman, J., Vancanneyt, G., and Mithen, R. (2004). Glucosinolate and amino acid biosynthesis in Arabidopsis. Plant Physiol 135:828-839.
Gigolashvili, T., Berger, B., Mock, H. P., Muller, C., Weisshaar, B., and Flugge, U. I. (2007a). The transcription factor HIG1/MYB51 regulates indolic glucosinolate biosynthesis in Arabidopsis thaliana. Plant J.: 886-901.
Gigolashvili, T., Yatusevich, R., Berger, B., Muller, C., and Flugge, U. I. (2007b). The R2R3-MYB transcription factor HAG1/MYB28 is a regulator of methionine-derived glucosinolate biosynthesis in Arabidopsis thaliana. Plant J.
Gray, J. (2005). Guard cells: transcription factors regulate stomatal movements. Curr. Biol. 15:R593-R595.
Grubb, C. D. and Abel, S. (2006). Glucosinolate metabolism and its control. Trends Plant Sci. 11:89-100.
Halkier, B. A. and Gershenzon, J. (2006). Biology and Biochemistry of Glucosinolates. Annu. Rev. Plant Biol. 57:303-333.
Hansen, B. G., Kliebenstein, D. J., and Halkier, B. A. (2007). Identification of a flavin monoxygenase as the S-oxygenating enzyme in aliphatic glucosinolate biosynthesis in Arabidopsis. Plant J.: 902-910.
Hirai, M. Y., Sugiyama, K., Sawada, Y., Tohge, T., Obayashi, T., Suzuki, A., Araki, R., Sakurai, N., Suzuki, H., Aoki, K., Goda, H., Nishizawa, O. I., Shibata, D., and Saito, K. (2007). Omics-based identification of Arabidopsis Myb transcription factors regulating aliphatic glucosinolate biosynthesis. Proc. Natl. Acad. Sci. U.S.A 104:6478-6483.
Irizarry, R. A., Hobbs, B., Collin, F., Beazer-Barclay, Y. D., Antonellis, K. J., Scherf, U., and Speed, T. P. (2003). Exploration, normalization, and summaries of high density oligonucleotide array probe level data. Biostatistics. 4:249-264.
Jansen, R. C. and Nap, J. P. (2004). Regulating gene expression: surprises still in store. Trends Genet. 20:223-225.
Jansen, R. C. and Nap, J. P. (2001). Genetical genomics: the added value from segregation. Trends in Genetics 17:388-391.
Jin, H., Cominelli, E., Bailey, P., Parr, A., Mehrtens, F., Jones, J., Tonelli, C., Weisshaar, B., and Martin, C. (2000). Transcriptional repression by AtMYB4 controls production of UV-protecting sunscreens in Arabidopsis. EMBO J. 19:6150-6161.
Kim, J. H., Durrett, T. P., Last, R. L., and Jander, G. (2004). Characterization of the Arabidopsis TU8 glucosinolate mutation, an allele of TERMINAL FLOWER2. Plant Mol. Biol. 54:671-682.
Kliebenstein, D., Pedersen, D., Barker, B., and Mitchell-Olds, T. (2002). Comparative analysis of quantitative trait loci controlling glucosinolates, myrosinase and insect resistance in Arabidopsis thaliana. Genetics 161:325-332.
Kliebenstein, D. J., D'Auria, J. C., Behere, A. S., Kim, J. H., Gunderson, K. L., Breen, J. N., Lee, G., Gershenzon, J., Last, R. L., and Jander, G. (2007). Characterization of seed-specific benzoyloxyglucosinolate mutations in Arabidopsis thaliana. Plant J.
Kliebenstein, D. J., Kroymann, J., Brown, P., Figuth, A., Pedersen, D., Gershenzon, J., and Mitchell-Olds, T. (2001b). Genetic control of natural variation in Arabidopsis glucosinolate accumulation. Plant Physiol 126:811-825.
Kliebenstein, D. J., Kroymann, J., and Mitchell-Olds, T. (2005). The glucosinolate-myrosinase system in an ecological and evolutionary context. Curr. Opin. Plant Biol. 8:264-271.
Kliebenstein, D. J., Lambrix, V. M., Reichelt, M., Gershenzon, J., and Mitchell-Olds, T. (2001c). Gene duplication in the diversification of secondary metabolism: tandem 2-oxoglutarate-dependent dioxygenases control glucosinolate biosynthesis in Arabidopsis. Plant Cell 13:681-693.
Kliebenstein, D. J., West, M. A., van, L. H., Kim, K., Doerge, R. W., Michelmore, R. W., and St Clair, D. A. (2006a). Genomic survey of gene expression diversity in Arabidopsis thaliana. Genetics 172:1179-1189.
Kliebenstein, D. J., West, M. A., van, L. H., Loudet, O., Doerge, R. W., and St Clair, D. A. (2006b). Identification of QTLs controlling gene expression networks defined a priori. BMC. Bioinformatics 7:308.
Lehfeldt, C., Shirley, A. M., Meyer, K., Ruegger, M. O., Cusumano, J. C., Viitanen, P. V., Strack, D., and Chapple, C. (2000). Cloning of the SNG1 gene of Arabidopsis reveals a role for a serine carboxypeptidase-like protein as an acyltransferase in secondary metabolism. Plant Cell 12:1295-1306.
Levy, M., Wang, Q., Kaspi, R., Parrella, M. P., and Abel, S. (2005). Arabidopsis IQD1, a novel calmodulin-binding nuclear protein, stimulates glucosinolate accumulation and plant defense. Plant J. 43:79-96.
Loudet, O., Chaillou, S., Camilleri, C., Bouchez, D., and niel-Vedele, F. (2002). Bay-0× Shahdara recombinant inbred line population: a powerful tool for the genetic dissection of complex traits in Arabidopsis. Theor. Appl. Genet. 104:1173-1184.
Lukowitz, W., Gillmor, C. S., and Scheible, W. R. (2000). Positional Cloning in Arabidopsis. Why It Feels Good to Have a Genome Initiative Working for You. Plant Physiol. 123:795-806.
Martin, C., Bhatt, K., Baumann, K., Jin, H., Zachgo, S., Roberts, K., Schwarz-Sommer, Z., Glover, B., and Perez-Rodrigues, M. (2002). The mechanics of cell fate determination in petals. Philos. Trans. R. Soc. Lond B Biol. Sci. 357:809-813.
Mewis, I., Appel, H. M., Hom, A., Raina, R., and Schultz, J. C. (2005). Major signaling pathways modulate Arabidopsis glucosinolate accumulation and response to both phloem-feeding and chewing insects. Plant Physiol 138:1149-1162.
Mikkelsen, M. D., Petersen, B. L., Glawischnig, E., Jensen, A. B., Andreasson, E., and Halkier, B. A. (2003). Modulation of CYP79 genes and glucosinolate profiles in Arabidopsis by defense signaling pathways. Plant Physiol 131:298-308.
Mithen, R., Faulkner, K., Magrath, R., Rose, P., Williamson, G., and Marquez, J. (2003). Development of isothiocyanate-enriched broccoli, and its enhanced ability to induce phase 2 detoxification enzymes in mammalian cells. Theor. Appl. Genet. 106:727-734.
Petersen, B. L., Chen, S., Hansen, C. H., Olsen, C. E., and Halkier, B. A. (2002). Composition and content of glucosinolates in developing Arabidopsis thaliana. Planta 214:562-571.
Ramsay, N. A. and Glover, B. J. (2005). MYB-bHLH-WD40 protein complex and the evolution of cellular diversity. Trends Plant Sci. 10:63-70.
Rosso, M. G., Li, Y., Strizhov, N., Reiss, B., Dekker, K., and Weisshaar, B. (2003). An Arabidopsis thaliana T-DNA mutagenized population (GABI-Kat) for flanking sequence tag-based reverse genetics. Plant Mol. Biol. 53:247-259.
Schadt, E. E., Monks, S. A., Drake, T. A., Lusis, A. J., Che, N., Colinayo, V., Ruff, T. G., Milligan, S. B., Lamb, J. R., Cavet, G., Linsley, P. S., Mao, M., Stoughton, R. B., and Friend, S. H. (2003). Genetics of gene expression surveyed in maize, mouse and man. Nature 422:297-302.
Sinlapadech, T., Stout, J., Ruegger, M. O., Deak, M., and Chapple, C. (2007). The hyper-fluorescent trichome phenotype of the brt1 mutant of Arabidopsis is the result of a defect in a sinapic acid: UDPG glucosyltransferase. Plant J. 49:655-668.
Skirycz, A., Reichelt, M., Burow, M., Birkemeyer, C., Rolcik, J., Kopka, J., Zanor, M. I., Gershenzon, J., Strnad, M., Szopa, J., Mueller-Roeber, B., and Witt, I. (2006). DOF transcription factor AtDof1.1 (OBP2) is part of a regulatory network controlling glucosinolate biosynthesis in Arabidopsis. Plant J. 47:10-24.
Textor, S., Bartram, S., Kroymann, J., Falk, K. L., Hick, A., Pickett, J. A., and Gershenzon, J. (2004). Biosynthesis of methionine-derived glucosinolates in Arabidopsis thaliana: recombinant expression and characterization of methylthioalkylmalate synthase, the condensing enzyme of the chain-elongation cycle. Planta 218:1026-1035.
Textor, S., de Kraker, J. W., Hause, B., Gershenzon, J., and Tokuhisa, J. G. (2007). MAM3 Catalyzes the Formation of All Aliphatic Glucosinolate Chain Lengths in Arabidopsis. Plant Physiol. 144:60-71.
Tissier, A. F., Marillonnet, S., Klimyuk, V., Patel, K., Torres, M. A., Murphy, G., and Jones, J. D. G. (1999). Multiple Independent Defective Suppressor-mutator Transposon Insertions in Arabidopsis: A Tool for Functional Genomics. Plant Cell 11:1841-1852.
Wang, S., Basten, C. J., and Zeng, Z.-B. (2006). Windows QTL Cartographer 2.5. Department of Statistics, North Carolina State University, Raleigh, N.C.
Wentzell, A., Rowe, H., Hansen, B., Ticconi, C., Halkier, B., and Kliebenstein, D. (2007). Linking Metabolic QTL with Network and cis-eQTL Controlling Biosynthetic Pathways. PLoS Genetics:doi:10.1371/journal.pgen.0030162.eor.
West, M. A., Kim, K., Kliebenstein, D. J., van, L. H., Michelmore, R. W., Doerge, R. W., and St Clair, D. A. (2007). Global eQTL mapping reveals the complex genetic architecture of transcript-level variation in Arabidopsis. Genetics 175:1441-1450.
West, M. A., van, L. H., Kozik, A., Kliebenstein, D. J., Doerge, R. W., St Clair, D. A., and Michelmore, R. W. (2006). High-density haplotyping with microarray-based expression and single feature polymorphism markers in Arabidopsis. Genome Res. 16:787-795.
Zeng, Z. B., Kao, C. H., and Basten, C. J. (1999). Estimating the genetic architecture of quantitative traits. Genet. Res. 74:279-289.
Zhu, J., Verslues, P. E., Zheng, X., Lee, B. H., Zhan, X., Manabe, Y., Sokolchik, I., Zhu, Y., Dong, C. H., Zhu, J. K., Hasegawa, P. M., and Bressan, R. A. (2005). HOS10 encodes an R2R3-type MYB transcription factor essential for cold acclimation in plants. Proc. Natl. Acad. Sci. U.S.A 102:9966-9971.

### Sequence Annex Index

1 AT1G62560 mRNA
2 AT1G62560 amino acid translation
3 AT1G65860 mRNA
4 AT1G65860 amino acid translation
5 AT1G62570 mRNA
6 AT1G62570 amino acid translation
7 AT1G62540 mRNA
8 AT1G62540 amino acid translation
9 AT1G12140 mRNA
10 AT1G12140 amino acid translation

### Sequence Annexes

| | |
|---|---|
| DEFINITION | Arabidopsis thaliana disulfide oxidoreductase/monooxygenase |
| | (AT1G62560) mRNA, complete cds. |

| FEATURES | | Location/Qualifiers |
|---|---|---|
| | source | 1 . . . 1576 |
| | | /organism = “Arabidopsis thaliana” |
| | | /mol_type = “mRNA” |
| | | /db_xref = “taxon: 3702” |
| | | /chromosome = “1” |
| | | /ecotype = “Columbia” |
| | gene | 1 . . . 1576 |
| | | /locus_tag = “AT1G62560” |
| | | /note = “synonyms: T3P18.12, T3P18_12” |
| | | /db_xref = “GeneID: 842553” |
| | CDS | 90 . . . 1478 |
| | | /locus_tag = “AT1G62560” |
| | | /go_component = “chloroplast” |
| | | /go_function = “disulfide oxidoreductase activity; |
| | | monooxygenase activity” |
| | | /go_process = “electron transport” |
| | | /note = “flavin-containing monooxygenase family protein/ |
| | | FMO family protein, similar to flavin-containing |
| | | monooxygenase GB:AAA21178 GI: 349534 SP\|P32417 from |
| | | (Oryctolagus cuniculus); contains Pfam profile PF00743 |
| | | Flavin-binding monooxygenase-like” |
| | | /codon_start = 1 |
| | | /product = “disulfide oxidoreductase/monooxygenase” |
| | | /protein_id = “NP 176444.1” |
| | | /db_xref = “GI: 15221491” |
| | | /db_xref = “GeneID: 842553” |

| /translation = “MAPAQNQITSHKVAVIGAGPAGLITSRELRREGHSVVVFEREKQVGGLWVYTPKSDSDPLSL | | |
|---|---|---|
| DPTRSKVHSSIYESLRTWNPRESMGVRDFPFLPRFDDESRDARRYPNHREVLAYIQDFAREFKIEEMIRFETEVVRVE | | |
| PVDNGNWRVQSKNSGGFLEDEIYDAVVVCNGHYTEPNIAHIPGIKSWPGKQIHSHNYRVPDPFENEVVVVIGNFASGA | | |
| DISRDIAKVAKEVHIASRAREPHTYEKISVPQNNLWMHSEIDTTHEDGSIVFKNGKVIFADSIVYCTGYKYNFPFLET | | |
| NGYLRIDEKRVEPLYKHVFPPALAPGLAFVGLPAMGIVFVMFEIQSKWVAAVLSGRVTLPSTDKMMEDINAWYASLDA | | |
| LGIPKRHTHTIGRIQSEYLNWVAKESGCELVERWRGQEVDGGYLRLVAHPETYRDEWDDDELIEEAYNDFSRKKLISV | | |
| DPSYYLENGT” | | |
| ORIGIN | | |
| | 1 | atcttgccat taaaatatag tatttatatt tggcctgaag ctgatgcaac ttatacacaa |
| | 61 | aacctactat tattaagatt tgacaaaata tggcaccagc tcaaaaccaa atcacttcta |
| | 121 | aacacgtggc agtgatcgga gccggaccag ccggtctcaL aacgtctagg gagctccgtc |
| | 181 | gtgaaggtca cagtgtagtt gtgtttgaac gggagaaaca agtcggtggt ctatgggttt |
| | 241 | acacacctaa atccgattcc gatccactta gccttgaccc cacccgatcc aaagtccact |
| | 301 | cgagcatcta cgagtctctc cgaaccaatg tcccgagaga aagtatgggt gtcagggact |
| | 361 | tcccgttttt gccacgtttc gatgacgagt caagagacgc gagacgttat ccaaatcata |
| | 421 | gggaagttct tgcgtatatt caagactttg ctagagagtt taaaatagag gagatgatcc |
| | 481 | ggttcgagac cgaggtggtt cgcgttgaac cggttgacaa cgggaactgg agggtccagt |
| | 541 | cgaaaaactc cggcgggttc ttggaagatg agatctatga cgccgtcgtg gtttgcaatg |
| | 601 | gtcactatac agaaccaaat attgctcata ttcctggtat aaaatcgtgg ccaggaaagc |
| | 661 | agattcatag ccacaactat agagttcctg atccattcga aaacgaggtg gtggtggtga |
| | 721 | taggaaattt tgcgagtggt gccgatatta gtagggacat agctaaggtc gcaaaagaag |
| | 781 | tccacattgc gtctagagca agggaacccc acacatacga gaagatttcc gttccccaaa |
| | 841 | acaatctatg gatgcattcc gaaatcgaca ccacccatga ggatgggtcg attgttttca |
| | 901 | aaaacgggaa ggtgatattt gctgatagca ttgtgtattg caccgggtac aagtataact |
| | 961 | tcccatttct tgaaacaaat ggctatttgc gcattgatga aaaacgtgtt gaacctctat |
| | 1021 | acaagcatgt ctttccacca gcgcttgccc ctggacttgc tttcgttggt ttgccagcaa |
| | 1081 | tggggatagt atttgttatg tttgaaatcc aaagcaaatg ggtggcagca gtcttgtcag |
| | 1141 | gacgagttac acttccctca acagataaga tgatggaaga tattaatgcg tggtatgcgt |
| | 1201 | cgcttgatgc cttaggtatt cccaagagac atactcatac gataggtaga attcagagtg |
| | 1261 | agtacctcaa ttgggtcgcg aaagaatctg gttgtgaact cgtagaacgt tggagaggtc |
| | 1321 | aagaagttga cggcggatac ctgagacttg tggcccatcc agaaacttac cgtgatgaat |
| | 1381 | gggacgacga tgaactcata gaagaagcgt acaatgattt ttctaggaag aagttgatta |
| | 1441 | gtgttgatcc ttcttattac ctcgaaaatg gaagatgatc tgcgccaata gtgccgactt |
| | 1501 | gtttttcttt tctggtaggt gggttgattc caagccttca ataaattgca aaactattgt |
| | 1561 | aagctttaca atttac |

| | |
|---|---|
| DEFINITION | Arabidopsis thaliana disulfide oxidoreductase/monooxygenase |
| | (AT1G65860) mRNA, complete cds. |

| FEATURES | | Location/Qualifiers |
|---|---|---|
| | source | 1 . . . 1591 |
| | | /organism = “Arabidopsis thaliana” |
| | | /mol_type = “mRNA” |
| | | /db_xref = “taxon: 3702” |
| | | /chromosome = “1” |
| | | /ecotype = “Columbia” |
| | gene | 1 . . . 1591 |
| | | /locus_taq = “AT1G65860” |
| | | /note = “synonyms: F12P19.2, F12P19_2” |
| | | /db_xref = “GeneID: 842897” |
| | CDS | 68 . . . 1447 |
| | | /locus_tag = “AT1G65860” |
| | | /go_component = “cellular component unknown” |
| | | /go_function = “disulfide oxidoreductase activity; |
| | | monooxygenase activity” |
| | | /go_process = “electron transport” |
| | | /note = “flavin-containing monooxygenase family protein/ |
| | | FMO family protein, similar to flavin-containing |
| | | monooxygenase FMO3 (dimethylaniline monoxygenase (N-oxide |
| | | forming) 3) GI: 349533 (SP\|P32417) from Oryctolagus |
| | | cuniculus, (SP\|P97501) from Mus musculus; contains Pfam |
| | | profile PF00743 Flavin-binding monooxygenase-like domain” |
| | | /codon_start = 1 |
| | | /product = “disulfide oxidoreductase/monooxygenase” |
| | | /protein_id = “NP 176761.1” |
| | | /db_xref = “GI: 15218834” |
| | | /db_xref = “GeneID: 842897” |

| /translation = “MAPTQNTICSKHVAVIGAGAAGLVTARELRREGHTVVVFDREKQVGGLWNYSSKADSDPLSL | | |
|---|---|---|
| DTTRTIVHTSIYESLRTNLPRECMGFTDFPFVPRIHDISRDSRRYPSHREVLAYLQDFAREFKIEEMVRFETEVVCVE | | |
| PVNGKWSVRSKNSVGFAAHEIFDAVVVCSGHFTEPNVAHIPGIKSWPGKQIHSHNYRVPGPFNNEVVVVIGNYASGAD | | |
| ISRDIAKVAKEVHIASRASESDTYQKLPVPQNNLWVHSIEDFAHQDGSILFKNGKVVYADTIVHCTGYKYYFPFLETN | | |
| GYININENRVEPLYKHVFLPALAPSLSFIGLPGMAIQFVMFEIQSKWVAAVLSGRVILPSQDKMMEDIIEWYATLDVL | | |
| GIPKRHTHKLGIISCEYLNWIAEECHCSPVENWRIQEVERGFQRMVSHPEIYRDEWDDDDLMEEAYKDFARKKLISSH | | |
| PSYFLES” | | |
| ORIGIN | | |
| | 1 | aaaacatatt gtttcacatt cctaaataaa tgaaaatcaa acatatcata gaaatttaat |
| | 61 | aaaataaatg gcaccaactc aaaacacaat ctgttcgaaa cacgtggcag tgattggagc |
| | 121 | cggagctgcc ggtctcgtaa cggctaggga acttcgtcgt gaaggtcaca ctgtcgttgt |
| | 181 | ctttgaccgg gaaaaacaag tgggaggtct ctggaactac tcatctaaag ctgactctga |
| | 241 | cccgcttagc ctcgacacaa cccgaaccat agtccacacg agcatctacg agtctctccg |
| | 301 | aaccaacctc ccgagagaat gtatgggttt tacggacttt cctttcgtgc cacgcattca |
| | 361 | tgacatctcg agagactcga gacggtatcc gagtcacaga gaagttcttg cgtatcttca |
| | 421 | agactttgct agagagttta aaatagagga gatggtccgg ttcgagacag aggtggtttg |
| | 481 | tgLtgagccg gttaacggga aatggagtgt ccggtccaag aattccgttg gtttcgccgc |
| | 541 | ccatgaaatc tttgatgccg tcgttgtttg tagtggtcac tttacagaac ctaacgttgc |
| | 601 | tcatattcct gggataaaat cgtggccagg aaagcagatc catagccaca actacagagt |
| | 661 | tcctggtcca ttcaataacg aggtagtggt ggtgatcgga aattatgcga gcggtgctga |
| | 721 | tattagtagg gatatagcta aggtcgcgaa agaagttcac attgcctcta gagcgagtga |
| | 781 | atctgatacg taccagaagc ttccagtgcc ccaaaacaat ctatgggttc attccgagat |
| | 841 | agacttcgcc catcaggatg gatccattct tttcaaaaat gggaaggtgg tatatgctga |
| | 901 | taccattgtg cattgcactg ggtacaaata ttactttcca tttcttgaaa ccaatggcta |
| | 961 | tataaacatt aatgaaaacc gcgtcgaacc tctatacaag catgtctttc tacccgcgct |
| | 1021 | agcccccagt ctttctttca tcggtttacc tggaatggcc atacaattcg ttatgtttga |
| | 1081 | aattcaaagc aaatgggtgg ctgcagtctt gtccggacga gttatacttc cctcgcaaga |
| | 1141 | caagatgatg gaagatatta ttgagtggta tgcaacgctt gatgtgttag gaattcccaa |
| | 1201 | aagacatacg cataaattgg gtaaaatttc gtgtgagtac ctcaactgga tcgcggaaga |
| | 1261 | atgtcattgt tcgccagttg aaaattggag aattcaagaa gttgagcgtg gattccagag |
| | 1321 | aatggtctcc cacccagaaa tttaccgcga tgaatgggat gatgatgaLc ttatggaaga |
| | 1381 | agcgtacaag gattttgcta ggaagaagtt aattagttct catccttctt atttcctcga |
| | 1441 | atcatgatga tgatctgcga caaatattgt ccaaaaatta aaaatcgctt gtttcgttct |
| | 1501 | ttcttatagt cttaagtagc agctggactt gttttttaat tttgtttgtg tgttccagta |
| | 1561 | acttaaagtt gatactctta tttatgttca t |

| | |
|---|---|
| DEFINITION | Arabidopsis thaliana disulfide oxidoreductase/monooxygenase/ |
| | oxidoreductase (AT1G62570) mRNA, complete cds. |

| FEATURES | | Location/Qualifiers |
|---|---|---|
| | source | 1 . . . 1779 |
| | | /organism = “Arabidopsis thaliana” |
| | | /mol_type = “mRNA” |
| | | /db_xref = “taxon: 3702” |
| | | /chromosome = “1” |
| | | /ecotype = “Columbia” |
| | gene | 1 . . . 1779 |
| | | /locus_tag = “AT1G62570” |
| | | /note = “synonyms: T3P18.13, T3P18_13” |
| | | /db_xref = “GeneID: 842554” |
| | CDS | 72 . . . 1457 |
| | | /locus_tag = “AT1G62570” |
| | | /go_component = “chloroplast” |
| | | /go_function = “disulfide oxidoreductase activity; |
| | | monooxygenase activity; oxidoreductase activity” |
| | | /go_process = “electron transport” |
| | | /note = “flavin-containing monooxygenase family protein/ |
| | | FMO family protein, low similarity to flavin-containing |
| | | monooxygenase FMO3 (Rattus norvegicus) GI: 12006730; |
| | | contains Pfam profile PF00743: Flavin-binding |
| | | monooxygenase-like” |
| | | /codon_start = 1 |
| | | /product = “disulfide oxidoreductase/monooxygenase/ |
| | | oxidoreductase” |
| | | /protein_id = “NP 564797.1” |
| | | /db_xref = “GI: 18407612” |
| | | /db_xref = “GeneID: 842554” |

| /translation = “MAPAPSPINSQHVAVIGAGAAGLVAARELRREGHTVVVLDREKQVGGLWVYTPETESDELGL | | |
|---|---|---|
| DPTRPIVHSSVYKSLRTNLPRECMGYKDFPFVPRGDDPSRDSRRYPSHREVLAYLQDFATEFNIEEMIRFETEVLRVE | | |
| PVNGKWRVQSKTGGGFSNDEIYDAVVMCCGHFAEPNIAQIPGIESWPGRQTHSHSYRVPDPFKDEVVVVIGNFASGAD | | |
| ISRDISKVAKEVHIASRASKSNTFEKRPVPNNNLWMHSEIDTAHEDGTIVFKNGKVVHADTIVHCTGYKYYFPFLETN | | |
| NYMRVDDNRVEPLYKHIFPPALAPGLSFIGLPAMGLQFYMFEVQSKWVAAVLSGRVTLPSVDEMMDDLKLSYETQEAL | | |
| GIPKRYTHKLGKSQCEYLDWIADLCGFPHVEHWRDQEVTRGYQRLGNQPETFRDEWDDDDLMEEAYEDFARLNLINFH | | |
| PSRFLESGR” | | |
| ORIGIN | | |
| | 1 | acacaacaat ccttcttaca tttctaccaa caaaacacaa aacacaaaca tagcattcaa |
| | 61 | aactttgaaa aatggcacca gctcctagtc caatcaattc tcaacacgtg gcggtgatcg |
| | 121 | gagccggagc agccggttta gtagaagcca gagagcttcg tcgtgaaggt cacaccgtcg |
| | 181 | ttgtccttga ccgagagaaa caagtaggtg gtctttgggt ttacacacct gaaaccgagt |
| | 241 | ccgacgagct tggtcttgac ccgacccgac ccatagtcca ctcgagcgtc tacaagtctc |
| | 301 | tccgaaccaa tctccctaga gaatgtatgg gttacaagga tttccctttc gtgccacgtg |
| | 361 | gcgatgatcc gtcaagagac tctagaaggt atccgagtca cagggaagtt cttgcgtacc |
| | 421 | ttcaagactt tgctacagag tttaacatag aggagatgat ccggttcgag actgaggttc |
| | 481 | ttcgtgttga accggttaat ggtaaatgga gggtccagtc taaaaccggc ggcggttttt |
| | 541 | ccaacgatga gatctatgac gccgttgtaa tgtgttgtgg acatttcgca gaaccaaaca |
| | 601 | tcgctcaaat tcctggaatt gagtcatggc cggggaggca aacacacagc cacagttatc |
| | 661 | gagttcctga tccattcaaa gatgaggtgg tggtagtaat cgggaatttt gcgagtggag |
| | 721 | ccgatatcag tagagacata tctaaagtcg caaaagaagt tcatatcgca tctagagcaa |
| | 781 | gtaaatccaa cactttcgaa aaacgtcctg tacctaataa caatctctgg atgcactctg |
| | 841 | agatagacac cgcccacgag gatggtacca ttgtttttaa aaatgggaag gtggtacatg |
| | 901 | ctgataccat tgtccattgt accgggtaca agtattactt tccatttctt gagaccaata |
| | 961 | attatatgag agttgatgac aatcgcgttg aacctctcta caagcatatt tttccacctg |
| | 1021 | cgctagctcc cggactttct ttcattggtt tacctgcaat gggtctacaa ttctatatgt |
| | 1081 | ttgaagtcca aagcaaatgg gttgctgcag tcttgtctgg acgagttaca cttccttcgg |
| | 1141 | tagatgaaat gatggacgat cttaagttgt cgtatgaaac acaagaagcg ttaggtattc |
| | 1201 | ccaaaagata tacacataag ttgggtaaat ctcagtgtga gtacctcgat tggatcgcag |
| | 1261 | acctgtgtgg attcccacat gttgaacatt ggagagatca agaagtaact cgcggttacc |
| | 1321 | agagacttgg taatcaacca gaaactttcc gtgatgaatg ggatgatgat gatctcatgg |
| | 1381 | aagaagcata cgaagatttt gctagactaa atctgatcaa ttttcatcct tctcgttttc |
| | 1441 | tcgaatccgg aagatgaagt ttgactacga ttgtaattgt gtctacttgt ttggatttaa |
| | 1501 | agtacattgc attataaaaa taatgtgtga gtaaatagtt tataagagtg tgaaggtctt |
| | 1561 | cttggctagg gttacatgtt gttcgatctc cggaattagc ttcatggtgt ctagaaactt |
| | 1621 | ttgtttttta gaccaatatg ttaagaataa aagtatgtag ttaattcccg taagttttta |
| | 1681 | tgaatccctg gttcattgtg caaatgtttt tttttttgtt attgttctgt taatatcaaa |
| | 1741 | gagtgtcctt aaatgtatgc ataattccct ctttttggc |

| | |
|---|---|
| DEFINITION | Arabidopsis thaliana disulfide oxidoreductase/monooxygenase/ |
| | oxidoreductase (AT1062540) mRNA, complete cds. |

| FEATURES | | Location/Qualifiers |
|---|---|---|
| | source | 1 . . . 1740 |
| | | /organism = “Arabidopsis thaliana” |
| | | /mol_type = “mRNA” |
| | | /db_xref = “taxon: 3702” |
| | | /chromosome = “1” |
| | | /ecotype = “Columbia” |
| | gene | 1 . . . 1740 |
| | | /locus_tag = “AT1G62540” |
| | | /note = “synonyms: T3P18.10, T3P18_10” |
| | | /db_xref = “GeneID: 842551” |
| | CDS | 77 . . . 1450 |
| | | /locus_tag = “AT1G62540” |
| | | /go_component = “endomembrane system” |
| | | /go_function = “disulfide oxidoreductase activity; |
| | | monooxygenase activity; oxidoreductase activity” |
| | | /go_process = “electron transport” |
| | | /note = “flavin-containing monooxygenase family protein/ |
| | | FMO family protein, similar to flavin-containing |
| | | monooxygenase GB: AAA21178 GI: :349534 from Oryctolagus |
| | | cuniculus (SP\|P32417), SP\|297501 from Mus musculus; |
| | | contains Pfam profile PF00743 Flavin-binding |
| | | monooxygenase-like” |
| | | /codon_start = 1 |
| | | /product = “disulfide oxidoreductase/monooxygenase/ |
| | | oxidoreductase” |
| | | /protein_id = “NP 564796.1” |
| | | /db_xref = “GI: 18407608” |
| | | /db_xref = “GeneID: 842551” |

| /translation = “MAPAQNPISSQHVVVIGAGAAGLVAARELSREGHTVVVLEREKEVGGLWIYSPKAESDPLSL | | |
|---|---|---|
| DPTRSIVHSSVYESLRTNLPRECMGFTDFPFVPRFDDESRDSRRYPSHMEVLAYLQDFAREFNLEEMVRFEIEVVRVE | | |
| PVNGKWRVWSKTSGGVSHDEIFDAVVVCSGHYTEPNVAHIPGIKSWPGKQIHSHNYRVPGPFENEVVVVIGNFASGAD | | |
| ISRDIAKVAKEVHIASRASEFDTYEKLPVPRNNLWIHSEIDTAYEDGSIVFKNGKVVYADSIVYCTGYKYRFTFLETN | | |
| GYMNIDENRVEHLYKHVFPPALSPGLSFVGLPSMGIQFVMFEIQSKWVAAVLSRRVTLPTEDKMMEDISAWYASLDAV | | |
| GIPKRYTHKLGKIQSEYLNWVAEECGCPLVEHWRNQQIVRGYQRLVSHPETYRDEWDDNDLMEEAYEDFARKKLISFH | | |
| PSHIL” | | |
| | 1 | taaccaaaac acagagatca tacgacagtc tcctaccaaa taaagaaaaa tccaccatac |
| | 61 | cataaagttc aaatatatgg caccagctca aaacccgatc agttctcaac acgtggtagt |
| | 121 | catcggagcc ggagcagccg gtctcgtagc ggctagggag ctcagtcgtg aaggtcacac |
| | 181 | tgttgtcgta ttagagcggg agaaagaagt aggaggtctc tggatctatt cacccaaagc |
| | 241 | cgaatccgac ccgcttagcc ttgacccaac ccgttccata gtccactcga gcgtgtacga |
| | 301 | gtcLctccga accaacctcc cacgagaatg tatgggtttc acggacttcc cttttgtgcc |
| | 361 | tcgtttcgat gacgagtcaa gagactcgag acggtatccg agccacatgg aagttcttgc |
| | 421 | gtaccttcaa gactttgcta gagagtttaa cctagaggag atggttcggt tegagatega |
| | 481 | ggtggttcgg gttgaaccgg ttaacgggaa atggagggtc tggtctaaaa cctctggcgg |
| | 541 | tgtttcccac gatgagatct Ltgacgccgt tgttgtttgc agtggacact atacagaacc |
| | 601 | aaacgttgct catattcctg gtataaaatc gtggccagga aagcagatcc atagccacaa |
| | 661 | ctacagagtt cctgggccat tcgaaaacga ggtggtggtg gtcatcggaa attttgctag |
| | 721 | cggtgccgat attagtaggg acatagctaa ggtcgcgaaa gaagttcaca ttgcatctag |
| | 781 | agcgagtgaa tttgatacat acgaaaagct tcccgtgcct cggaacaatc tatggattca |
| | 841 | ttcggaaata gacacggcat atgaagatgg gtccattgtt ttcaaaaacg ggaaggtggt |
| | 901 | atatgctgat agcattgtgt attgcactgg atataaatat cgcttcacat tccttgaaac |
| | 961 | caatggctat atgaacattg atgaaaaccg cgtagaacat ctatacaagc atgtatttcc |
| | 1021 | acctgcgctt tctcctggtc tttcattcgt tggtttacca tcgatgggca tacaatttgt |
| | 1081 | tatgtttgaa atccaaagca aatgggtggc agcagtcttg tcaaggcggg ttacacttcc |
| | 1141 | cacagaagat aagatgatgg aagatattag tgcgtggtat gcatcgcttg atgcggtagg |
| | 1201 | cattcctaaa agatatacac ataaattggg taaaattcag agtgagtacc tcaattgggt |
| | 1261 | cgcagaagaa tgtggttgtc cgctcgttga acattggaga aatcaacaaa tcgtccgcgg |
| | 1321 | ataccagaga cttgtctcac acccagaaac ttatcgcgat gaatgggacg acaatgacct |
| | 1381 | tatggaagaa gcttacgagg actttgctag gaagaaatta attagtttcc atccttccca |
| | 1441 | tatcctctaa tcaagaaaat gatttttgtg tttttacttt gggggtgggt gtattgtatt |
| | 1501 | taagaagcat aaggaaggat ggattctttc cttttcaggg ttgattgcta aactattgaa |
| | 1561 | agctttgaat aaataggagg gtttatctct aaggcatgat gccctgattg ttatttttct |
| | 1621 | ttgtgtgtgt ttgtttttgt ttgcatttga gtttttattt attttgtgct tatgtttgaa |
| | 1681 | ttttacactg attatgttca ccacgtatag atgcaaatat tacttccgtt tcttgaaacc |

| | |
|---|---|
| DEFINITION | Arabidopsis thaliana disulfide oxidoreductase/monooxygenase |
| | (AT1G12140) mRNA, complete cds. |

| FEATURES | | Location/Qualifiers |
|---|---|---|
| | source | 1 . . . 1573 |
| | | /organism = “Arabidopsis thaliana” |
| | | /moltype = “mRNA” |
| | | /db_xref = “taxon: 3702” |
| | | /chromosome = “1” |
| | | /ecotype = “Columbia” |
| | gene | 1 . . . 1573 |
| | | /locus_tag = “AT1012140” |
| | | /note = “synonyms: T28K15.12, T28K15_12” |
| | | /db_xref = “GeneID: 837766” |
| | CDS | 18 . . . 1397 |
| | | /locus_tag = “AT1G12140” |
| | | /go_component = “mitochondrion” |
| | | /go_function = “disulfide oxidoreductase activity; |
| | | monooxygenase activity” |
| | | /go_process = “electron transport” |
| | | /note = “flavin-containing monooxygenase family protein/ |
| | | FMO family protein, similar to flavin-containing |
| | | monooxygenase (Cavia porcellus) GI: 191259; contains Pfam |
| | | profile PF00743: Flavin-binding monooxygenase-like” |
| | | /codon_start = 1 |
| | | /product = “disulfide oxidoreductase/ monooxygenase” |
| | | /protein_id = “NP 172678.3” |
| | | /db_xref = “GI: 42561939” |
| | | /db_xref = “GeneID: 837766” |

| /translation = “MAPARTRVNSLNVAIGAGAAGLVAARELRRENHTVVVFERDSKVGGLWVYTPNSEPDPLSLD | | |
|---|---|---|
| PNRTIVHSSVYDSLRTNLPRECMGYRDFPFVPRPEDDESRDSRRYPSHREVLAYLEDFAREFKLVEMVRFKTEVVLVE | | |
| PEDKKWRVQSKNSDGISKDEIFDAVVVCNGHYTEPRVAHVPGIDSWPGKQIHSHNYRVPDQFKDQVVVVIGNFASGAD | | |
| ISRDITGVAKEVHIASRSNPSKTYSKLPGSNNLWLHSMIESVHEDGTIVFQNGKVVQADTIVHCTGYKYHFPFLNTNG | | |
| YITVEDNCVGPLYEHVFPPALAPGLSFIGLPWMTLQFFMFELQSKWVAAALSGRVTLPSEEKMMEDVTAYYAKREAFG | | |
| QPKRYTHRLGGGQVDYLNWIAEQIGAPPGEQWRYQEINGGYYRLATQSDTFRDKWDDDHLIVEAYEDFLRQKLISSLP | | |
| SQLLES” | | |
| ORIGIN | | |
| | 1 | atcatcacac aaaaaagatg gcaccagcac gaacccgagt caactcactc aacgtggcag |
| | 61 | tgatcggagc cggagccgcc ggactcgtag ctgcaagaga gctccgccgc gagaatcaca |
| | 121 | ccgtcgtcgt tttcgaacgt gactcaaaag tcggaggtct ctgggtatac acacctaaca |
| | 181 | gcgaaccaga cccgcttagc ctcgatccaa accgaaccat cgtccattca agcgtctatg |
| | 241 | attctctccg aaccaatctc ccacgagagt gcatgggtta cagagacttc cccttcgtgc |
| | 301 | ctcgacctga agatgacgaa tcaagagact cgagaaggta ccctagtcac agagaagttc |
| | 361 | ttgcttacct tgaagacttc gctagagaat tcaaacttgt ggagatggtt cgatttaaga |
| | 421 | ccgaagtagt tcttgtcgag cctgaagata agaaatggag ggttcaatcc aaaaattcag |
| | 481 | atgggatctc caaagatgag atctttgatg ctgttgttgt ttgtaatgga cattatacag |
| | 541 | aacctagagt tgctcatgtt cctggtatag attcatggcc agggaagcag attcatagcc |
| | 601 | acaattaccg tgttcctgat caattcaaag accaggtggt ggtagtgata ggaaattttg |
| | 661 | cgagtggagc tgatatcagc agggacataa cgggagtggc taaagaagtc catatcgcgt |
| | 721 | ctagatcgaa tccatctaag acatactcaa aacttcccgg gtcaaacaat ctatggcttc |
| | 781 | actctatgat agaaagtgta cacgaagatg ggacgattgt ttttcagaac ggtaaggttg |
| | 841 | tacaagctga taccattgtg cattgcactg gttacaaata tcacttccca tttctcaaca |
| | 901 | ccaatggcta tattactgtt gaggataact gtgttggacc gctttacgaa catgtctttc |
| | 961 | cgcctgcgct tgctcccggg ctttccttca tcggtttacc ctggatgaca ctgcaattct |
| | 1021 | ttatgtttga gctccaaagc aagtgggtgg ctgcagcttt gtctggccgg gtcacacttc |
| | 1081 | cttcagaaga gaaaatgatg gaagacgtta ccgcctacta tgcaaagcgt gaggctttcg |
| | 1141 | ggcaacctaa gagatacaca catcgacttg gtggaggtca ggttgattac cttaattgga |
| | 1201 | tagcagagca aattggtgca ccgcccggtg aacaatggag atatcaggaa ataaatggcg |
| | 1261 | gatactacag acttgctaca caatcagaca ctttccgtga taagtgggac gatgatcatc |
| | 1321 | tcatagttga ggcttatgag gatttcttga gacagaagct gattagtagt cttccttctc |
| | 1381 | agttattgga atcttgaaga tcatgaataa ttccttgaac aaatgattga cctgtctgtg |
| | 1441 | tgttgttgta ttgttctttg ttgttgtgtt aaataaaagc cgtcaaggtt tcattgtctt |
| | 1501 | tttttttatc tttgaatgtt tggaaaaaaa aacaaggttt tatacaaaat gaaatcatca |
| | 1561 | ctaagcaagt tgt |

Sequence Annex Index (continued)
11 Myb28, CDS nucleotide
12 Myb28—amino acid translation
13 Myb29, CDS nucleotide
14 Myb29—amino acid translation
15 Myb76, CDS nucleotide
16 Myb76—amino acid translation

### 17 Myb28 for 35Senh-construct

### 18 Myb29 for 35Senh-construct

### 19 Myb76, for 35Senh-construct

35Senhancer sequence

>Myb28, CDS NCBI), 1101 bp
ATGTCAAGAAAGCCATGTTGCGTCGGAGAAGGCTTGAAGAAAGGAGCATG
GACCACCGAGGAGGACAAGAAACTCATCTCTTACATCCACGACCACGGCG
AGGGAGGCTGGCGCGACATTCCCCAAAAAGCTGGGTTGAAACGGTGTGGA
AAGAGTTGTAGACTGCGATGGACCAACTACCTTAAACCTGAGATCAAAAG
AGGCGAGTTTAGTTCAGAGGAAGAGCAGATTATCATCATGCTTCATGCTT
CTCGTGGCAACAAGTGGTCGGTCATAGCGAGACATTTACCTAGAAGAACA
GACAACGAGATCAAGAACTACTGGAACACGCATCTCAAAAAACGTTTGAT
GGAACAGGGTATTGATCCCGTGACTCACAAGCCACTGGCTTCTAGTTCCA
ACCCTACGGTCGATGAGAATTTGAATTCCCCAAATGCCTCTAGTTCCGAC
AAGCAATACTCCCGATCGAGCTCAATGCCTTTTCTGTCTCGTCCTCCTCC
ATCCAGTTGCAACATGGTTTCCAAGGTCTCCGAGCTTAGCAGCAATGATG
GGACACCGATTCAAGGCAGTTCCTTGAGTTGCAAGAAACGTTTCAAGAAA
TCAAGTTCTACATCAAGGCTCTTGAACAAAGTTGCGGCTAAGGCCACTTC
CATCAAAGATATATTGTCGGCTTCCATGGAAGGTAGCTTGAGTGCTACTA
CAATATCACATGCAAGCTTTTTTAATGGCTTCACTGAGCAGATTCGCAAT
GAAGAGGATAGTTCTAACACATCCCTGACAAATACTCTTGCTGAATTTGA
TCCCTTCTCCCCATCATCGTTGTACCCCGAACATGAGATCAATGCTACTT
CTGATCTCAACATGGACCAAGATTACGATTTTTCACAATTTTTCGAAAAA
TTCGGAGGAGATAACCACAATGAGGAGAACAGTATGAATGATCTCCTTAT
GTCCGATGTTTCCCAAGAAGTCTCATCAACTAGCGTTGATGATCAAGACA
ATATGGTAGGAAACTTCGAGGGATGGTCAAATTATCTTCTTGACCATACC
AATTTTATGTATGACACCGACTCAGACTCGCTTGAAAAGCATTTCATATG
A
/translation = “MSRKPCCVGEGLKKGAWTTEEDKKLISYIHDHGE
GGWRDIPQKAGLKRCGKSCRLRWTNYLKPEIKRGEFSSEEEQIIIMLHAS
RGNKWSVIARHLPRRTDNEIKNYWNTHLKKRLMEQGIDPVTHKPLASSSN
PTVDENLNSPNASSSDKQYSRSSSMPFLSRPPPSSCNMVSKVSELSSNDG
TPIQGSSLSCKKRFKKSSSTSRLLNKVAAKATSIKDILSASMEGSLSATT
ISHASFFNGFTEQIRNEEDSSNTSLTNTLAEFDPFSPSSLYPEHEINATS
DLNMDQDYDFSQFFEKFGGDNHNEENSMNDLLMSDVSQEVSSTSVDDQDN
MVGNFEGWSNYLLDHTNFMYDTDSDSLEKHFI”
>Myb29, CDS (TAIR) 1011 bp
ATGTCAAGAAAGCCATGTTGTGTGGGAGAAGGACTGAAGAAAGGAGCATG
GACTGCCGAAGAAGACAAGAAACTCATCTCTTACATTCATGAACACGGTG
AAGGAGGCTGGCGTGACATTCCCCAAAAAGCTGGACTAAAACGATGTGGA
AAGAGTTGTAGATTGCGATGGGCTAACTATTTGAAACCTGACATCAAGAG
AGGAGAGTTTAGCTATGAGGAGGAACAGATTATCATCATGCTACACGCTT
CTCGCGGCAACAAGTGGTCAGTCATAGCGAGACATTTGCCCAAAAGAACA
GATAACGAGATTAAGAACTACTGGAACACGCATCTCAAAAAGCTCCTGAT
CGATAAGGGAATCGATCCCGTGACCCACAAGCCACTTGCCTATGACTCAA
ACCCGGATGAGCAATCGCAATCGGGTTCCATCTCTCCAAAGTCTCTTCCT
CCTTCAAGCTCCAAAAATGTACCGGAGATAACCAGCAGTGACGAGACACC
GAAATATGATGCTTCCTTGAGCTCCAAGAAACGTTGTTTTAAGAGATCGA
GTTCTACATCAAAACTGTTAAACAAAGTTGCAGCTAGGGCTTCTTCCATG
GGAACTATACTAGGCGCCTCCATCGAAGGAACCTTGATCAGCTCTACACC
GTTGTCTTCATGTCTAAATGATGACTTTTCTGAAACAAGTCAATTTCAGA
TGGAAGAATTTGATCCATTCTATCAGTCATCTGAACACATAATTGATCAT
ATGAAAGAAGATATCAGCATCAACAATTCCGAATACGATTTCTCGCAGTT
TCTCGAGCAGTTTAGTAACAACGAAGGGGAAGAAGCTGACAATACTGGAG
GAGGATATAACCAAGATCTTCTTATGTCTGATGTCTCATCAACAAGCGTT
GATGAAGACGAGATGATGCAAAACATAACTGGTTGGTCAAATTATCTCCT
TGACCATTCCGATTTCAATTATGACACGAGCCAAGATTACGACGACAAGA
ACTTCATATGA
/translation = “MSRKPCCBGEGLKKGAWTAEEDKKLISYIHEHGE
GGWRDIPQKAGLKRCGKSCRLRWANYLKPDIKRGEFSYEEEQIIIMLHAS
RGNKWSVIARHLPKRTDNEIKNYWNTHLKKLLIDKGIDPVTHKPLAYDSN
PDEQSQSGSISPKSLPPSSSKNVPEITSSDETPKYDASLSSKKRCFKRSS
STSKLLNKVAARASSMGTILGASIEGTLISSTPLSSCLNDDFSETSQFQM
EEFDPFYQSSEHIIDHMKEDISINNSEYDFSQFLEQFSNNEGEEADNTGG
GYNQDLLMSDVSSTSVDEDEMMQNITGWSNYLLDHSDFNYDTSQDYDDKNFI”
>Myb76, CDS, (TAIR) 1017 bp
ATGTCAAAGAGACCATATTGTATCGGAGAAGGACTGAAGAAAGGAGCATG
GACTACAGAAGAGGATAAAAAACTCATCTCTTATATCCACGACCACGGTG
AAGGAGGCTGGCGTGACATTCCAGAAAAAGCTGGGCTGAAACGGTGTGGA
AAGAGTTGTAGATTACGGTGGACTAACTATTTGAAACCAGATATCAAGAG
AGGAGAGTTTAGCTATGAGGAAGAGCAGATTATCATCATGCTTCATGCAT
CTCGTGGCAATAAGTGGTCTGTCATAGCTAGACATTTGCCAAAAAGAACG
GATAACGAGGTCAAAAACTATTGGAACACACATCTCAAGAAACGTTTAAT
CGATGATGGCATTGATCCCGTGACACACAAGCCACTAGCTTCTTCTAACC
CTAATCCAGTTGAGCCCATGAAGTTCGATTTCCAAAAGAAATCCAATCAG
GATGAGCACTCTTCACAGTCTAGTTCTACAACTCCAGCATCTCTTCCCCT
TTCCTCGAATTTGAACAGTGTTAAATCCAAAATTAGCAGTGGTGAGACGC
AGATAGAAAGTGGTCACGTGAGCTGCAAGAAACGTTTTGGACGATCGAGC
TCTACATCAAGGTTGTTAAACAAAGTTGCAGCTAGAGCTTCTTCCATCGG
CAACATCTTATCAACATCCATAGAAGGAACCTTGAGATCTCCTGCATCAT
CTTCAGGACTCCCAGACTCGTTCTCTCAATCATATGAGTACATGATCGAT
AACAAAGAAGATCTCGGTACGAGCATTGATCTCAACATCCCCGAGTATGA
TTTCCCACAGTTTCTTGAGCAACTCATTAACGATGACGACGAAAATGAGA
ACATTGTTGGGCCCGAACAAGATCTCCTTATGTCCGATTTCCCATCAACA
TTCGTTGATGAAGACGATATACTTGGAGACATAACCAGTTGGTCAACTTA
TCTTCTTGACCATCCCAATTTTATGTATGAATCGGATCAAGATTCCGACG
AGAAGAACTTCTTATGA
/translation = “MSKRYPCIGEGLKKGAWTTEEDKKLSIYIHDHGE
GGWRDIPEKAGLKRCGKSCRLRWTNYLKPDIKRGEFSYEEEQIIIMLHAS
RGNKWSVIARHLPKRTDNEVKNYWNTHLKKRLIDDGIDPVTHKPLASSNP
NPVEPMKFDFQKKSNQDEHSSQSSSTTPASLPLSSNLNSVKSKISSGETQ
IESGHVSCKKRFGRSSSTSRLLNKVAARASSIGNILSTSIEGTLRSPASS
SGLPDSFSQSYEYMIDNKEDLGTSIDLNIPEYDFPQFLEQLINDDDENEN
IVGPEQDLLMSDFPSTFVDEDDILGDITSWSTYLLDHPNFMYESDQDSDEKNFL”
>Myb28, primer 60 & 61, for 35Senh-construct
caatgtaaatgctcggaagtgagtcgttgcgaaaatttaggtttgtaaaa
tgaaggattatggtgagttttagtttgcaaaataactaaaatattatggg
accaaggaaataatcaagaataagtgaagatacactatgggaccgtttaa
gtaggttgacatatataactgactggaaccagcggatcttagggatataa
tcaatacttattgactaaaattttcccaaaagaaagaagaatcaaatgat
tactctatgtagtaacccaaactgatcctaacaaaattgtagaaatgcag
atggtttaaatatgtggcgctctcataaaactcctacttcaggtaatctt
tttacacagtttggagctatcgtagctcttaacattttcactccagcaat
gactagaaccaacagaacaatgagagattggcttctatccatagaaagct
tcaacacgaaaaccgaccaaaacgaaatgttaaacccaagccttcttcaa
gcatagctgtatcatattctatcttccttgtaagagttccttttgttaaa
aactaaatactaaatccgacttaaagaataataatcaagaacttcaaaat
agcaaagtaaaatatacacacgcacaaattgataagagttcacttagctt
gcagtacgagaactaggcaggggcagacctagcttaagagtgtaggtgtg
gcaggtgtttaattatatagaatttactttgtggcactaacatatttttg
ttttataatgcaaaataagatgttaaatttgattaaatttatatacaata
caagtttgtgttctatgtaaaatatttttctagatcaaacaaggtctagt
tttaaacgatccatgggagtataaattttatctttttcactctaccttga
aaaatgcgcatgagataaaatcataggtacatatacatacgtgaagaata
gcatcagaaaatattgttctaactattccgataactaacaaaaccttagg
aaacctcatcaagactagttcgaattaaatcataaggtttaggttgagag
agtcaaagagggaatgatataaatagaagaatattttttgtttaagaatg
atttttagacaatggaaagaagaatatgttaaggtggtatagacgacgag
caataatacaacagccacaaaagtggcaaacaaaaaggacctacgctgga
aaaaaaacacgtgatgttacaatcaccctttcattctcaatgatgaacaa
taatgtttattattgataagaaaaacaataatgtaaatttatactttctc
gttaaccagatttgttttttcattatgcgtttgcagtataaaaatagtaa
aatacgttttaagtattaaactgtttgatagtttttttttatatatatac
ctaacagaaaccaactatttaaacaatacaaaaatatctgcaaagatata
tatataatacaatcgaattcttaaaagttatatatatttgcaaacgtccc
tttagttattcccctccaactctccatgttggatcaatcattcaattttt
ttttaataaccaaaagttaaatgtacaaatatgcaagaacctacaggtac
gtttacgtgatatataaattaaaatattgcatctcgtaccgaagcgcatt
accgtatttaaaatacctgaaagtaggaaaatatagtactatacaaacac
cacttttcggacattattttcatagaaaagttacgaattatcctttttaa
ctattgatctatttaaataatttactaaccataactatcttgttacgttt
tcacaaaaaaaaaaaaaaaaaaatctcattacgtacgtgtatatatatgg
aatagctcataacctcaccactaccacagaaatcatgcctcttggttctt
ttccataagcttataacatatattttttttaaaatctactctgcgttaaa
aaaatgaaaacacgtagcagcagtgtgggtaagatcaaagggtgtttctc
gatcagtttcatattcagatgtatcagagttctcattaacagatctgttt
ctttttccttatctgattaaacaatttccttcagaattttacttttttga
acatatatagtttttctctgttcctatatcttgagttttgtgagaggtta
attatatgaaattttacgcattattgttcatctatatcgaaaaacaatgt
caagaaagccatgttgcgtcggagaaggcttgaagaaaggagcatggacc
accgaggaggacaagaaactcatctcttacatccacgaccacggcgaggg
aggctggcgcgacattccccaaaaagctggtttatacaaatctatacata
cactcatttttgtacttgttgtagaaaattgttctgataaacatattgtg
tctgattagggttgaaacggtgtggaaagagttgtagactgcgatggacc
aactaccttaaacctgagatcaaaagaggcgagtttagttcagaggaaga
gcagattatcatcATGCTTCATGCTTCTCGTGGCAACAAgtacgtttcta
tgtttctatgtgtgtgcgtggaccctcgaatgtgaaatgaatttcatgaa
aaagttttcatataatatttattatgtagacataatcatcattttaatct
tggtctccgatctatcttattttctttagGTGGTCGGTCATAGCGAGACA
TTTACCTAGAAGAACAGACAACGAGATCAAGAACTACTGAACCCTACGGT
CGATGAGAATTTGAATTCCCCAAATGCCTCTAGTTCCGACAAGCAATACT
CCCGATCGAGCTCAATGCCTTTTCTGTCTCGTCCTCCTCCATCCAGTTGC
AACATGGTTTCCAAGGTCTCCGAGCTTAGCAGCAATGATGGGACACCGAT
TCAAGGCAGTTCCTTGAGTTGCAAGAAACGTTTCAAGAAATCAAGTTCTA
CATCAAGGCTCTTGAACAAAGTTGCGGCTAAGGCCACTTCCATCAAAGAT
ATATTGTCGGCTTCCATGGAAGGTAGCTTGAGTGCTACTACAATATCACA
TGCAAGCTTTTTTAATGGCTTCACTGAGCAGATTCGCAATGAAGAGGATA
GTTCTAACACATCCCTGACAAATACTCTTGCTGAATTTGATCCCTTCTCC
CCATCATCGTTGTACCCCGAACATGAGATCAATGCTACTTCTGATCTCAA
CATGGACCAAGATTACGATTTTTCACAATTTTTCGAAAAATTCGGAGGAG
ATAACCACAATGAGGAGAACAGTATGAATGATCTCCTTATGTCCGATGTT
TCCCAAGAAGTCTCATCAACTAGCGTTGATGATCAAGACAATATGGTAGG
AAACTTCGAGGGATGGTCAAATTATCTTCTTGACCATACCAATTTTATGT
ATGACACCGACTCAGACTCGCTTGAAAAGCATTTCATATGAgtcttcata
tccaaacagaaaggtttcaaactattcgacgacttaaaataatggttctg
tacccaaggttagtcgattactaactcgctcgaacgagatattgtgtatg
tattaattagtatttgggttgtttactatatgtccaaggcgtgtttatta
cgatgttaaacaagggttaatcttaacacttaagtttccccaagaataaa
taaaatagggtttgagttagggtttctcttacattgagaaccatgcatgt
aacctcgcgaatcaattggtaattgatttgtgcgggccacgatgtttata
ctaatatttctttctaaagcttgttttatttatcttatttcgtagtagta
cttcccatt
>Myb29, primer 68 & 59, for the 35Senh construct
attttcaacgattgcgttgtttcccaaattatgaattggaactttggtag
catcgcaatatatacgacgtttgggtttggcccatgctgccatgcatagc
aatagttttaaatacatgtggttggtaatatagaaaatggtttgaaagac
cacaaactttgatcagtgatcgattcggtagggccacaaataacaatgtt
ttcgccacatggtcatactttacgttttcgatgagaaaatatttaaacag
tttgttcgtcaaatttcgattaactagaacaaaattcataaacgagagag
acagaataattcgagagagctagagtgagggtaactagaaagatagtaac
tgattttgtatctaataattaattcattaatttaaaatcaatgataaatc
actttgatggttgtggccttgtgggtaattataattaacacgtaccattt
tttatcaaggcatttttaaacattttgtttgtttttattgaagttttctt
ctcactattcaaaaacgtaaaaccctaacaaaaaaaagtaaaagtagaac
tgtttacaagtctggctgaatgggttgattgactcgacaaaagattttcc
atgtggattaatagaacaaattttaataatatatacgaaattgatgtatt
ttcttttctttcgatcactattaatgtcttaaataataaaaacatatagt
acattttacagattataaatttatgttgtgttttattttgagttttggct
tgaattttttttttttttttgttgttgttgttaaagtggtttgaatctgt
attggttacaaataaaacaaataatgttacgttcattttgtctgtagata
tttttcttacaacttatgcagctatctttggggtttcattttgagtgtgg
atgtttggttttgagttaactctgcatgttccgaagaggcgtaactaaaa
taaaagaaactacttgagatgcgagatgtgaaatgtgattagatgagaaa
aaacgaacattaataattgagcaaaataactttatttaaattttgaattc
agcgttagtgttacacccaaagtggcaaacaaataggaccgatgttgaaa
aagaacacacgtgatataaaatgtactgagagaaaattatttgcattaga
tgacaataaatacaataataatgaatagatgaaataacttttagttgacg
aaaaaaaaaaaaaacttttaatctattttattcactagatcaaaaagcat
gtttcagacagttttattcttatcattcaattatttcacaacgtataatt
ttagtttattttcgtaatttgttaatatacgtatcaattgaatatttttg
acggtttttattatgtcatttaattatttaagggaacatagtttatttta
aaatgcagttctattttacaaaaaaaaagaaaaaaaaaattgcagttcta
cgttgacatctagctgatcaactattcatcatatatacttgtataatcta
ttattttaagttctatattattattaatatgttaaatatagatatatcta
tttaagaaaatatcatataaatatatgttataaatctatatatagaaaaa
ataaagcacagaattttgtcccacattctgtcgatacgtactcgagctta
tgaagttgttcttttctaattatattttttcccattgccctttatcaaat
caactctaataaaaatatatggtaacttatgaagttgtcatgtatttatg
atatttctctttgggtcggcactgtatttgtgatgttgattatttatcta
gtggcagaaaatattccataagtctctctcaaaccatttgaatagttcca
aaaacatcttgtcactaacactcactcttgatgagttttttttttttttt
tttggggggtcaaagtactcttgatgatgagttgatattcttatttaaaa
aaagcttattacttatttaagttatttcaaaaagtacattctacacgagt
gccaggcttatatatatgcataaacatatataattatgcatggaggagta
gtagcttgcaatgtcttgaaactttgatatatcttctcctagtctttctt
ttaaatgtttaatatgaaaacacaaaatcctacaacggtcgtctaccaca
gtttctcagtcagtttcatattcagatgcatcagagttctcatcaagaga
tctatcagtctattgccttaaactcgacgacattctgttttttttttctt
ttcttattttttctttttcttatttcttacctataggttgtatgtaaatc
tatatcaaaaaaagaagaaaaacaagATGTCAAGAAAGCCATGTTGTGTG
GGAGAAGGACTGAAGAAAGGAGCATGGACTGCCGAAGAAGACAAGAAACT
CATCTCTTACATTCATGAACACGGTGAAGGAGGCTGGCGTGACATTCCCC
AAAAAGCTGgtatatatgtgctttattattatgtatatattttaaaacac
tttttacatatatataactataattgttgtttttatgacaaatgatggtg
tttagGACTAAAACGATGTGGAAAGAGTTGTAGATTGCGATGGGCTAACT
ATTTGAAACCTGACATCAAGAGAGGAGAGTTTAGCTATGAGGAGGAACAG
ATTATCATCATGCTACACGCTTCTCGCGGCAACAAgtaaaatcctagctt
gccgaaatccatataaataagggtatatataattaacacattattaaagt
ttatatatatgttttacttaaaagGTGGTCAGTCATAGCGAGACATTTGC
CCAAAAGAACAGATAACGAGATTAAGAACTACTGGAACACGCATCTCAAA
AAGCTCCTGATCGATAAGGGAATCGATCCCGTGACCCACAAGCCACTTGC
CTATGACTCAAACCCGGATGAGCAATCGCAATCGGGTTCCATCTCTCCAA
AGTCTCTTCCTCCTTCAAGCTCCAAAAATGTACCGGAGATAACCAGCAGT
GACGAGACACCGAAATATGATGCTTCCTTGAGCTCCAAGAAACGTTGTTT
TAAGAGATCGAGTTCTACATCAAAACTGTTAAACAAAGTTGCAGCTAGGG
CTTCTTCCATGGGAACTATACTAGGCGCCTCCATCGAAGGAACCTTGATC
AGCTCTACACCGTTGTCTTCATGTCTAAATGATGACTTTTCTGAAACAAG
TCAATTTCAGATGGAAGAATTTGATCCATTCTATCAGTCATCTGAACACA
TAATTGATCATATGAAAGAAGATATCAGCATCAACAATTCCGAATACGAT
TTCTCGCAGTTTCTCGAGCAGTTTAGTAACAACGAAGGGGAAGAAGCTGA
CAATACTGGAGGAGGATATAACCAAGATCTTCTTATGTCTGATGTCTCAT
CAACAAGCGTTGATGAAGACGAGATGATGCAAAACATAACTGGTTGGTCA
AATTATCTCCTTGACCATTCCGATTTCAATTATGACACGAGCCAAGATTA
CGACGACAAGAACTTCATATGAtccgttgattgcttaccggactagagtt
gaccggttaatgtcatatggttctcttagatatttgtcaagttatagtaa
aggtccactatagggtcactatatattaatattcagtaatggattctctt
agttagagaaccttgtgatgccgtggatcaattagtatttgatttgcggg
agacacgagttttttttccttctattgttgtttgtggatttacgtactat
aaataataaataaaacacccatttgattgcaagcgttcactgtactaaaa
ccatttgatttaaagtttgagcc
>Myb for 35Senh construct
aagcgttcactgtactaaaaccatttgatttaaagtttgagccttagttt
gtctgacagtctgagccatgttaccaaaaacaatgaaaaatatgtaacac
attttaggtttttggtgatatgaaactccgaagaaacaaatccctactga
ctactgagaaagtcgataagcttttttgtggataagtttcatggatatat
tagaagtagtaaccattaaccaacaaaaaaatagcttaagtgagttatca
agggatcgatgaacaattatgagatccaatgtgtttttgttaagaggcaa
aatccgatgcagtctctatgagacaaaatttccatgggaaaaacagagag
ttctgaagtctctctaccttaaacatgtgcaagccttagcttcaaatgct
ccgtaaggttttcatttaaaaacatgaaataagatagagaaatgatactt
gatccaactgatgaagattaacaagataattttgaagcaacttctgtttg
tataatatgtcgtacaaaatctgctaccaatttagaggccaaattatttt
cttttctagacagtttgtgaggtgggcagctgaaggtgtttaagccaaga
ttctcaagatttataaatcttgaatcgaattaagctatcagccggaaatt
aggaaatgatatgcatatagggactaaagatatagttgttgcattaaaaa
gcttaaagagagaagtggatgtgaaaagaaaaaaaccacagatttttgca
cacaatcttgtgtgttgattgatatccaagtagactaattagactgcttt
gttctacacgataattggttgtttttagatatcaatacgaaacatgttaa
aatgtgaaaatattttagattagatgataacacctgaatttaatgacaaa
aaaaaaaaaaagtggatagagactagagggacagcaaggctgtgtgacat
atatgggcagatagacaaagaagccgaaaaacgtgcaccgtccaagattc
tggctactatacctaatttccttcccgcagggacttgacaaatatcacta
tctgccatttttagttttattttgtattggtgtcaaagaattgaaataat
gaacaacggtcgtaaaaagatgtaaatggtgtttgattgattaatgtttt
tttttttttcttagtatattttagcaattgcatattatcatataacatta
attaataattattgtgttgtagataaatgtcatgcataatgcagtaataa
atgtgtgtgcatatattatatatacacgttaattagatgctaaaatgagt
gacatatcttttaattctttgataacaccatttccataaatcattgtaaa
acttaccttataacaaaaaattaataaatgttataggggtcaattgaccc
ccataactctacactagccccacctctgcggataagcttaacatgtctat
taatattcattagtttacgtggtttaaaagtttattgtcacgagtgcatg
acacttaccgtgatgttgactatatgaagaggtagatcgtacgtgtacaa
atgacttcatagatctttgatcttttttttcttcttcttcttttttggta
atattctttagttttatttgatctattgtcgttgtaatgatctttgatta
caaaggaaaaaaaaaactaagacccttgacgaaaataataaccgtattcg
taatctctgatatcctacattatgtactatttctgatttttgtttcttat
acgcacttttgttctagatataactaagaaaATGTCAAAGAGACCATATT
GTATCGGAGAAGGACTGAAGAAAGGAGCATGGACTACAGAAGAGGATAAA
AAACTCATCTCTTATATCCACGACCACGGTGAAGGAGGCTGGCGTGACAT
TCCAGAAAAAGCTGgtacataactatatatagacgcatttgtgtctctat
aatatgaatttattcacaatctgttactaatatgtattaattattctctt
aattgatcatttgatctttatctgctttttttcgagtttagGGCTGAAAC
GGTGTGGAAAGAGTTGTAGATTACGGTGGACTAACTATTTGAAACCAGAT
ATCAAGAGAGGAGAGTTTAGCTATGAGGAAGAGCAGATTATCATCATGCT
TCATGCATCTCGTGGCAATAAgtacgtatggcatttctctaggcttgttt
gtgctcatatcagtttagtgaagacatgatcatcaatgttttgatatata
tgtaccctgtgtttttattttattttactagGTGGTCTGTCATAGCTAGA
CATTTGCCAAAAAGAACGGATAACGAGGTCAAAAACTATTGGAACACACA
TCTCAAGAAACGTTTAATCGATGATGGCATTGATCCCGTGACACACAAGC
CACTAGCTTCTTCTAACCCTAATCCAGTTGAGCCCATGAAGTTCGATTTC
CAAAAGAAATCCAATCAGGATGAGCACTCTTCACAGTCTAGTTCTACAAC
TCCAGCATCTCTTCCCCTTTCCTCGAATTTGAACAGTGTTAAATCCAAAA
TTAGCAGTGGTGAGACGCAGATAGAAAGTGGTCACGTGAGCTGCAAGAAA
CGTTTTGGACGATCGAGCTCTACATCAAGGTTGTTAAACAAAGTTGCAGC
TAGAGCTTCTTCCATCGGCAACATCTTATCAACATCCATAGAAGGAACCT
TGAGATCTCCTGCATCATCTTCAGGACTCCCAGACTCGTTCTCTCAATCA
TATGAGTACATGATCGATAACAAAGAAGATCTCGGTACGAGCATTGATCT
CAACATCCCCGAGTATGATTTCCCACAGTTTCTTGAGCAACTCATTAACG
ATGACGACGAAAATGAGAACATTGTTGGGCCCGAACAAGATCTCCTTATG
TCCGATTTCCCATCAACATTCGTTGATGAAGACGATATACTTGGAGACAT
AACCAGTTGGTCAACTTATCTTCTTGACCATCCCAATTTTATGTATGAAT
CGGATCAAGATTCCGACGAGAAGAACTTCTTATGAtctgtctatagatgg
cttgtcaatttcccaatgttga
>35Senhancer sequence
cttcgtcaacatggtggagcacgacacacttgtctactccaaaaatatca
aagatacagtctcagaagaccaaagggcaattgagacttttcaacaaagg
gtaatatccggaaacctcctcggattccattgcccagctatctgtcactt
tattgtgaagatagtggaaaaggaaggtggctcctacaaatgccatcatt
gcgataaaggaaaggccatcgttgaagatgcctctgccgacagtggtccc
aaagatggacccccacccacgaggagcatcgtggaaaaagaagacgttcc
aaccacgtcttcaaagcaagtggattgatgtgatatctcc

1. An isolated nucleic acid molecule which nucleic acid comprises a plant derived flavin-containing monooxygenases (FMO) nucleotide sequence encoding an FMO capable of catalysing oxidation of a thio- to a sulphinyl-group such as to form a sulphinylalkyl GSL.

2. (canceled)

3. A nucleic acid as claimed in claim 1 wherein the sulphinylalkyl GSL is a methlysulphinylalkyl GSL and wherein the alkyl is selected from the group consisting of propyl, butyl, hexyl, pentyl, heptyl, or octyl.

4. (canceled)

5. A nucleic acid as claimed in claim 1 wherein the FMO nucleotide sequence:
(i) encodes all or part of SEQ ID NO: 2, 4, 6, 8, or 10, or
(ii) encodes a variant FMO which is a homologous variant of SEQ ID NO 2 or 4 which shares at least about 65% identity therewith.

6. A nucleic acid as claimed in claim 5 wherein the FMO nucleotide sequence is selected from SEQ ID NO: 1, 3, 5, 7 or 9 or the genomic equivalent thereof.

7-13. (canceled)

14. A process for producing a nucleic acid which comprises a plant derived flavin-containing monooxygenases (FMO) nucleotide sequence encoding an FMO capable of catalysing oxidation of a thio- to a sulphinyl-group such as to form a sulphinylalkyl GSL,
wherein the FMO nucleotide sequence encodes a derivative of the amino acid sequence shown in SEQ ID NO: 2, 4, 6, 8, or 10 by way of addition, insertion, deletion or substitution of one or more amino acids, and the process comprises the step of modifying a nucleic acid as claimed in claim 5;
or the process comprises identifying or cloning a nucleic acid which is an allelic or other homologous or orthologous variant of the nucleotide sequence of claim 5, and the process employs all or part of a nucleic acid as claimed in claim 5 or the complement thereof.

15-17. (canceled)

18. A recombinant vector which comprises the nucleic acid of claim 5 wherein the nucleic acid is optionally operably linked to a promoter for transcription in a host cell, wherein the promoter is optionally an inducible promoter, and wherein the vector is optionally a plant vector or a microbial vector.

19-20. (canceled)

21. A method which comprises the step of introducing the vector of claim 18 into a host cell, and optionally causing or allowing recombination between the vector and the host cell genome such as to transform the host cell.

22. A host cell containing or transformed with a heterologous vector according to claim 21, wherein the host cell is either microbial or is a plant cell.

23-24. (canceled)

25. A method for producing a transgenic plant, which method comprises the steps of:
(a) performing a method as claimed in claim 22 wherein the host cell is a plant cell; and
(b) regenerating a plant from the transformed plant cell.

26. A transgenic plant which is obtainable by the method of claim 25, or which is a clone, or selfed or hybrid progeny or other descendant of said transgenic plant, which in each case includes a heterologous nucleic acid comprising a plant derived flavin-containing monooxygenases (FMO) nucleotide sequence encoding an FMO capable of catalysing oxidation of a thio- to a sulphinyl-group such as to form a sulphinylalkyl GSL.

27. A plant as claimed in claim 26 which further comprises a heterologous nucleic acid which comprises a plant derived MYB nucleotide sequence encoding a transcriptional regulator of a biosynthetic gene encoding a polypeptide with aliphatic GSL-biosynthetic or transport activity; and optionally a heterologous nucleic acid of the GS-Elong locus or GS-AOP locus,
wherein the plant is optionally selected from the list consisting of Brassica crop species (e.g. Brassica nigra, Brassica napus, Brassica oleraceae, Brassica rapa, Brassica carinata, Brassica juncea), cruciferous salads (e.g. Eruca sativa and Diplotaxis tenuifolia), and radish (Raphanus sativa).

28. (canceled)

29. An edible portion or propagule from a plant as claimed in claim 27.

30. An isolated polypeptide which is encoded by the FMO nucleotide sequence of claim 5.

31. A method of producing methylsulfinylalkyl GSL from the corresponding methylthioalkyl GSL (or desulfo-methylthioalkyl-GSL comprising incubating the recombinant FMO polypeptide of claim 30 with methylthioalkyl GSL (or desulfo-methylthioalkyl-GSL).

32-36. (canceled)

37. A method for influencing or affecting the aliphatic GSL-biosynthesis catalytic activity in a cell, the method comprising the step of causing or allowing expression of a heterologous nucleic acid as claimed in claim 5 within the cell.

38-39. (canceled)

40. A method for influencing or affecting the aliphatic GSL-biosynthesis or transport phenotype of a plant, which method comprises the step of:
i) causing or allowing expression of a heterologous nucleic acid as claimed in claim 5 within the cells of the plant, following an earlier step of introducing the nucleic acid into a cell of the plant or an ancestor thereof, or
ii) introducing a silencing agent capable of silencing expression of an FMO nucleotide sequence as described in claim 5 or an FMO nucleotide sequence which is an allelic or other homologous or orthologous variant of the nucleotide sequence of claim 5 into a cell of the plant or an ancestor thereof,
wherein the method optionally comprises any of the following steps of:
(i) causing or allowing transcription from a nucleic acid comprising the complement sequence of an FMO nucleotide sequence as described in claim 5 or of an FMO nucleotide sequence which is an allelic or other homologous or orthologous variant of the nucleotide sequence of claim 5 such as to reduce FMO or MYB expression by an antisense mechanism;
(ii) causing or allowing transcription from a nucleic acid encoding a stem loop precursor comprising 20-25 nucleotides, optionally including one or more mismatches, of an FMO nucleotide sequence as described in claim 5 or of an FMO nucleotide sequence which is an allelic or other homologous or orthologous variant of the nucleotide sequence of claim 5 such as to reduce FMO or MYB expression by an miRNA mechanism;
(iii) causing or allowing transcription from nucleic acid encoding double stranded RNA corresponding to 20-25 nucleotides, optionally including one or more mismatches, of an FMO nucleotide sequence as described in claim 5 or of an FMO nucleotide sequence which is an allelic or other homologous or orthologous variant of the nucleotide sequence of claim 5 such as to reduce FMO or MYB expression by an siRNA mechanism.

41-42. (canceled)

43. Double-stranded RNA which comprises an RNA sequence equivalent to part of an FMO nucleotide sequence as described in claim 5 or of an FMO nucleotide sequence which is an allelic or other homologous or orthologous variant of the nucleotide sequence of claim 5.

44-46. (canceled)

47. A method of producing a GSL, or modifying the production of a GSL, in a plant, which method comprises performing a method as claimed in claim 31 and optionally isolating the GSL from the plant.

48. A method of producing a GSL, or modifying the production of a GSL, in a fermentation tank, which method comprises introducing a cell according to claim 22 into the tank and culturing it, and optionally isolating the GSL from the tank, wherein the cell is selected from: bacterial, yeast filamentous fungi, or a plant cell in suspension culture.

49. A method for assessing the GSL phenotype of a plant, the method comprising the step of determining the presence and/or identity of a GSL-biosynthesis modifying allele therein comprising the use of a nucleic acid as claimed in claim 5 or part thereof to assess a GSL marker in the plant.

50. (canceled)

51. An isolated nucleic acid molecule which nucleic acid comprises a plant derived MYB nucleotide sequence encoding a transcriptional regulator of a biosynthetic gene encoding a polypeptide with aliphatic GSL-biosynthetic or transport activity wherein the MYB nucleotide sequence:
(i) encodes all or part of SEQ ID NO: 12, 14, or 16, or
(ii) encodes a variant MYB which is a homologous variant of SEQ ID NO 12, 14 or 16 which shares at least about 57% identity therewith.


SEQUENCE LISTING (57 sequences)

SEQ ID NO: 1 - DNA, 1576 bp, Arabidopsis thaliana
atcttgccattaaaatatagtatttatatttggcctgaagctgatgcaacttatacacaaaacctactattattaagatttgacaaaatatggcaccagctcaaaaccaaatcacttctaaacacgtggcagtgatcggagccggaccagccggtctcataacgtctagggagctccgtcgtgaaggtcacagtgtagttgtgtttgaacgggagaaacaagtcggtggtctatgggtttacacacctaaatccgattccgatccacttagccttgaccccacccgatccaaagtccactcgagcatctacgagtctctccgaaccaatgtcccgagagaaagtatgggtgtcagggacttcccgtttttgccacgtttcgatgacgagtcaagagacgcgagacgttatccaaatcatagggaagttcttgcgtatattcaagactttgctagagagtttaaaatagaggagatgatccggttcgagaccgaggtggttcgcgttgaaccggttgacaacgggaactggagggtccagtcgaaaaactccggcgggttcttggaagatgagatctatgacgccgtcgtggtttgcaatggtcactatacagaaccaaatattgctcatattcctggtataaaatcgtggccaggaaagcagattcatagccacaactatagagttcctgatccattcgaaaacgaggtggtggtggtgataggaaattttgcgagtggtgccgatattagtagggacatagctaaggtcgcaaaagaagtccacattgcgtctagagcaagggaaccccacacatacgagaagatttccgttccccaaaacaatctatggatgcattccgaaatcgacaccacccatgaggatgggtcgattgttttcaaaaacgggaaggtgatatttgctgatagcattgtgtattgcaccgggtacaagtataacttcccatttcttgaaacaaatggctatttgcgcattgatgaaaaacgtgttgaacctctatacaagcatgtctttccaccagcgcttgcccctggacttgctttcgttggtttgccagcaatggggatagtatttgttatgtttgaaatccaaagcaaatgggtggcagcagtcttgtcaggacgagttacacttccctcaacagataagatgatggaagatattaatgcgtggtatgcgtcgcttgatgccttaggtattcccaagagacatactcatacgataggtagaattcagagtgagtacctcaattgggtcgcgaaagaatctggttgtgaactcgtagaacgttggagaggtcaagaagttgacggcggatacctgagacttgtggcccatccagaaacttaccgtgatgaatgggacgacgatgaactcatagaagaagcgtacaatgatttttctaggaagaagttgattagtgttgatccttcttattacctcgaaaatggaagatgatctgcgccaatagtgccgacttgtttttcttttctggtaggtgggttgattccaagccttcaataaattgcaaaactattgtaagctttacaatttac

SEQ ID NO: 2 - protein, 462 aa, Arabidopsis thaliana
MAPAQNQITSKHVAVIGAGPAGLITSRELRREGHSVVVFEREKQVGGLWVYTPKSDSDPLSLDPTRSKVHSSIYESLRTNVPRESMGVRDFPFLPRFDDESRDARRYPNHREVLAYIQDFAREFKIEEMIRFETEVVRVEPVDNGNWRVQSKNSGGFLEDEIYDAVVVCNGHYTEPNIAHIPGIKSWPGKQIHSHNYRVPDPFENEVVVVIGNFASGADISRDIAKVAKEVHIASRAREPHTYEKISVPQNNLWMHSEIDTTHEDGSIVFKNGKVIFADSIVYCTGYKYNFPFLETNGYLRIDEKRVEPLYKHVFPPALAPGLAFVGLPAMGIVFVMFEIQSKWVAAVLSGRVTLPSTDKMMEDINAWYASLDALGIPKRHTHTIGRIQSEYLNWVAKESGCELVERWRGQEVDGGYLRLVAHPETYRDEWDDDELIEEAYNDFSRKKLISVDPSYYLENGR

SEQ ID NO: 3 - DNA, 1591 bp, Arabidopsis thaliana
aaaacatattgtttcacattcctaaataaatgaaaatcaaacatatcatagaaatttaataaaataaatggcaccaactcaaaacacaatctgttcgaaacacgtggcagtgattggagccggagctgccggtctcgtaacggctagggaacttcgtcgtgaaggtcacactgtcgttgtctttgaccgggaaaaacaagtgggaggtctctggaactactcatctaaagctgactctgacccgcttagcctcgacacaacccgaaccatagtccacacgagcatctacgagtctctccgaaccaacctcccgagagaatgtatgggttttacggactttcctttcgtgccacgcattcatgacatctcgagagactcgagacggtatccgagtcacagagaagttcttgcgtatcttcaagactttgctagagagtttaaaatagaggagatggtccggttcgagacagaggtggtttgtgttgagccggttaacgggaaatggagtgtccggtccaagaattccgttggtttcgccgcccatgaaatctttgatgccgtcgttgtttgtagtggtcactttacagaacctaacgttgctcatattcctgggataaaatcgtggccaggaaagcagatccatagccacaactacagagttcctggtccattcaataacgaggtagtggtggtgatcggaaattatgcgagcggtgctgatattagtagggatatagctaaggtcgcgaaagaagttcacattgcctctagagcgagtgaatctgatacgtaccagaagcttccagtgccccaaaacaatctatgggttcattccgagatagacttcgcccatcaggatggatccattcttttcaaaaatgggaaggtggtatatgctgataccattgtgcattgcactgggtacaaatattactttccatttcttgaaaccaatggctatataaacattaatgaaaaccgcgtcgaacctctatacaagcatgtctttctacccgcgctagcccccagtctttctttcatcggtttacctggaatggccatacaattcgttatgtttgaaattcaaagcaaatgggtggctgcagtcttgtccggacgagttatacttccctcgcaagacaagatgatggaagatattattgagtggtatgcaacgcttgatgtgttaggaattcccaaaagacatacgcataaattgggtaaaatttcgtgtgagtacctcaactggatcgcggaagaatgtcattgttcgccagttgaaaattggagaattcaagaagttgagcgtggattccagagaatggtctcccacccagaaatttaccgcgatgaatgggatgatgatgatcttatggaagaagcgtacaaggattttgctaggaagaagttaattagttctcatccttcttatttcctcgaatcatgatgatgatctgcgacaaatattgtccaaaaattaaaaatcgcttgtttcgttctttcttatagtcttaagtagcagctggacttgttttttaattttgtttgtgtgttccagtaacttaaagttgatactcttatttatgttcat

SEQ ID NO: 4 - protein, 459 aa, Arabidopsis thaliana
MAPTQNTICSKHVAVIGAGAAGLVTARELRREGHTVVVFDREKQVGGLWNYSSKADSDPLSLDTTRTIVHTSIYESLRTNLPRECMGFTDFPFVPRIHDISRDSRRYPSHREVLAYLQDFAREFKIEEMVRFETEVVCVEPVNGKWSVRSKNSVGFAAHEIFDAVVVCSGHFTEPNVAHIPGIKSWPGKQIHSHNYRVPGPFNNEVVVVIGNYASGADISRDIAKVAKEVHIASRASESDTYQKLPVPQNNLWVHSEIDFAHQDGSILFKNGKVVYADTIVHCTGYKYYFPFLETNGYININENRVEPLYKHVFLPALAPSLSFIGLPGMAIQFVMFEIQSKWVAAVLSGRVILPSQDKMMEDIIEWYATLDVLGIPKRHTHKLGKISCEYLNWIAEECHCSPVENWRIQEVERGFQRMVSHPEIYRDEWDDDDLMEEAYKDFARKKLISSHPSYFLES

SEQ ID NO: 5 - DNA, 1779 bp, Arabidopsis thaliana
acacaacaatccttcttacatttctaccaacaaaacacaaaacacaaacatagcattcaaaactttgaaaaatggcaccagctcctagtccaatcaattctcaacacgtggcggtgatcggagccggagcagccggtttagtagcagccagagagcttcgtcgtgaaggtcacaccgtcgttgtccttgaccgagagaaacaagtaggtggtctttgggtttacacacctgaaaccgagtccgacgagcttggtcttgacccgacccgacccatagtccactcgagcgtctacaagtctctccgaaccaatctccctagagaatgtatgggttacaaggatttccctttcgtgccacgtggcgatgatccgtcaagagactctagaaggtatccgagtcacagggaagttcttgcgtaccttcaagactttgctacagagtttaacatagaggagatgatccggttcgagactgaggttcttcgtgttgaaccggttaatggtaaatggagggtccagtctaaaaccggcggcggtttttccaacgatgagatctatgacgccgttgtaatgtgttgtggacatttcgcagaaccaaacatcgctcaaattcctggaattgagtcatggccggggaggcaaacacacagccacagttatcgagttcctgatccattcaaagatgaggtggtggtagtaatcgggaattttgcgagtggagccgatatcagtagagacatatctaaagtcgcaaaagaagttcatatcgcatctagagcaagtaaatccaacactttcgaaaaacgtcctgtacctaataacaatctctggatgcactctgagatagacaccgcccacgaggatggtaccattgtttttaaaaatgggaaggtggtacatgctgataccattgtccattgtaccgggtacaagtattactttccatttcttgagaccaataattatatgagagttgatgacaatcgcgttgaacctctctacaagcatatttttccacctgcgctagctcccggactttctttcattggtttacctgcaatgggtctacaattctatatgtttgaagtccaaagcaaatgggttgctgcagtcttgtctggacgagttacacttccttcggtagatgaaatgatggacgatcttaagttgtcgtatgaaacacaagaagcgttaggtattcccaaaagatatacacataagttgggtaaatctcagtgtgagtacctcgattggatcgcagacctgtgtggattcccacatgttgaacattggagagatcaagaagtaactcgcggttaccagagacttggtaatcaaccagaaactttccgtgatgaatgggatgatgatgatctcatggaagaagcatacgaagattttgctagactaaatctgatcaattttcatccttctcgttttctcgaatccggaagatgaagtttgactacgattgtaattgtgtctacttgtttggatttaaagtacattgcattataaaaataatgtgtgagtaaatagtttataagagtgtgaaggtcttcttggctagggttacatgttgttcgatctccggaattagcttcatggtgtctagaaacttttgttttttagaccaatatgttaagaataaaagtatgtagttaattcccgtaagtttttatgaatccctggttcattgtgcaaatgtttttttttttgttattgttctgttaatatcaaagagtgtccttaaatgtatgcataattccctctttttggc

SEQ ID NO: 6 - protein, 461 aa, Arabidopsis thaliana
MAPAPSPINSQHVAVIGAGAAGLVAARELRREGHTVVVLDREKQVGGLWVYTPETESDELGLDPTRPIVHSSVYKSLRTNLPRECMGYKDFPFVPRGDDPSRDSRRYPSHREVLAYLQDFATEFNIEEMIRFETEVLRVEPVNGKWRVQSKTGGGFSNDEIYDAVVMCCGHFAEPNIAQIPGIESWPGRQTHSHSYRVPDPFKDEVVVVIGNFASGADISRDISKVAKEVHIASRASKSNTFEKRPVPNNNLWMHSEIDTAHEDGTIVFKNGKVVHADTIVHCTGYKYYFPFLETNNYMRVDDNRVEPLYKHIFPPALAPGLSFIGLPAMGLQFYMFEVQSKWVAAVLSGRVTLPSVDEMMDDLKLSYETQEALGIPKRYTHKLGKSQCEYLDWIADLCGFPHVEHWRDQEVTRGYQRLGNQPETFRDEWDDDDLMEEAYEDFARLNLINFHPSRFLESGR

SEQ ID NO: 7 - DNA, 1740 bp, Arabidopsis thaliana
taaccaaaacacagagatcatacgacagtctcctaccaaataaagaaaaatccaccataccataaagttcaaatatatggcaccagctcaaaacccgatcagttctcaacacgtggtagtcatcggagccggagcagccggtctcgtagcggctagggagctcagtcgtgaaggtcacactgttgtcgtattagagcgggagaaagaagtaggaggtctctggatctattcacccaaagccgaatccgacccgcttagccttgacccaacccgttccatagtccactcgagcgtgtacgagtctctccgaaccaacctcccacgagaatgtatgggtttcacggacttcccttttgtgcctcgtttcgatgacgagtcaagagactcgagacggtatccgagccacatggaagttcttgcgtaccttcaagactttgctagagagtttaacctagaggagatggttcggttcgagatcgaggtggttcgggttgaaccggttaacgggaaatggagggtctggtctaaaacctctggcggtgtttcccacgatgagatctttgacgccgttgttgtttgcagtggacactatacagaaccaaacgttgctcatattcctggtataaaatcgtggccaggaaagcagatccatagccacaactacagagttcctgggccattcgaaaacgaggtggtggtggtcatcggaaattttgctagcggtgccgatattagtagggacatagctaaggtcgcgaaagaagttcacattgcatctagagcgagtgaatttgatacatacgaaaagcttcccgtgcctcggaacaatctatggattcattcggaaatagacacggcatatgaagatgggtccattgttttcaaaaacgggaaggtggtatatgctgatagcattgtgtattgcactggatataaatatcgcttcacattccttgaaaccaatggctatatgaacattgatgaaaaccgcgtagaacatctatacaagcatgtatttccacctgcgctttctcctggtctttcattcgttggtttaccatcgatgggcatacaatttgttatgtttgaaatccaaagcaaatgggtggcagcagtcttgtcaaggcgggttacacttcccacagaagataagatgatggaagatattagtgcgtggtatgcatcgcttgatgcggtaggcattcctaaaagatatacacataaattgggtaaaattcagagtgagtacctcaattgggtcgcagaagaatgtggttgtccgctcgttgaacattggagaaatcaacaaatcgtccgcggataccagagacttgtctcacacccagaaacttatcgcgatgaatgggacgacaatgaccttatggaagaagcttacgaggactttgctaggaagaaattaattagtttccatccttcccatatcctctaatcaagaaaatgatttttgtgtttttactttgggggtgggtgtattgtatttaagaagcataaggaaggatggattctttccttttcagggttgattgctaaactattgaaagctttgaataaataggagggtttatctctaaggcatgatgccctgattgttatttttctttgtgtgtgtttgtttttgtttgcatttgagtttttatttattttgtgcttatgtttgaattttacactgattatgttcaccacgtatagatgcaaatattacttccgtttcttgaaacc

SEQ ID NO: 8 - protein, 457 aa, Arabidopsis thaliana
MAPAQNPISSQHVVVIGAGAAGLVAARELSREGHTVVVLEREKEVGGLWIYSPKAESDPLSLDPTRSIVHSSVYESLRTNLPRECMGFTDFPFVPRFDDESRDSRRYPSHMEVLAYLQDFAREFNLEEMVRFEIEVVRVEPVNGKWRVWSKTSGGVSHDEIFDAVVVCSGHYTEPNVAHIPGIKSWPGKQIHSHNYRVPGPFENEVVVVIGNFASGADISRDIAKVAKEVHIASRASEFDTYEKLPVPRNNLWIHSEIDTAYEDGSIVFKNGKVVYADSIVYCTGYKYRFTFLETNGYMNIDENRVEHLYKHVFPPALSPGLSFVGLPSMGIQFVMFEIQSKWVAAVLSRRVTLPTEDKMMEDISAWYASLDAVGIPKRYTHKLGKIQSEYLNWVAEECGCPLVEHWRNQQIVRGYQRLVSHPETYRDEWDDNDLMEEAYEDFARKKLISFHPSHIL

SEQ ID NO: 9 - DNA, 1573 bp, Arabidopsis thaliana
atcatcacacaaaaaagatggcaccagcacgaacccgagtcaactcactcaacgtggcagtgatcggagccggagccgccggactcgtagctgcaagagagctccgccgcgagaatcacaccgtcgtcgttttcgaacgtgactcaaaagtcggaggtctctgggtatacacacctaacagcgaaccagacccgcttagcctcgatccaaaccgaaccatcgtccattcaagcgtctatgattctctccgaaccaatctcccacgagagtgcatgggttacagagacttccccttcgtgcctcgacctgaagatgacgaatcaagagactcgagaaggtaccctagtcacagagaagttcttgcttaccttgaagacttcgctagagaattcaaacttgtggagatggttcgatttaagaccgaagtagttcttgtcgagcctgaagataagaaatggagggttcaatccaaaaattcagatgggatctccaaagatgagatctttgatgctgttgttgtttgtaatggacattatacagaacctagagttgctcatgttcctggtatagattcatggccagggaagcagattcatagccacaattaccgtgttcctgatcaattcaaagaccaggtggtggtagtgataggaaattttgcgagtggagctgatatcagcagggacataacgggagtggctaaagaagtccatatcgcgtctagatcgaatccatctaagacatactcaaaacttcccgggtcaaacaatctatggcttcactctatgatagaaagtgtacacgaagatgggacgattgtttttcagaacggtaaggttgtacaagctgataccattgtgcattgcactggttacaaatatcacttcccatttctcaacaccaatggctatattactgttgaggataactgtgttggaccgctttacgaacatgtctttccgcctgcgcttgctcccgggctttccttcatcggtttaccctggatgacactgcaattctttatgtttgagctccaaagcaagtgggtggctgcagctttgtctggccgggtcacacttccttcagaagagaaaatgatggaagacgttaccgcctactatgcaaagcgtgaggctttcgggcaacctaagagatacacacatcgacttggtggaggtcaggttgattaccttaattggatagcagagcaaattggtgcaccgcccggtgaacaatggagatatcaggaaataaatggcggatactacagacttgctacacaatcagacactttccgtgataagtgggacgatgatcatctcatagttgaggcttatgaggatttcttgagacagaagctgattagtagtcttccttctcagttattggaatcttgaagatcatgaataattccttgaacaaatgattgacctgtctgtgtgttgttgtattgttctttgttgttgtgttaaataaaagccgtcaaggtttcattgtctttttttttatctttgaatgtttggaaaaaaaaacaaggttttatacaaaatgaaatcatcactaagcaagttgt

SEQ ID NO: 10 - protein, 459 aa, Arabidopsis thaliana
MAPARTRVNSLNVAVIGAGAAGLVAARELRRENHTVVVFERDSKVGGLWVYTPNSEPDPLSLDPNRTIVHSSVYDSLRTNLPRECMGYRDFPFVPRPEDDESRDSRRYPSHREVLAYLEDFAREFKLVEMVRFKTEVVLVEPEDKKWRVQSKNSDGISKDEIFDAVVVCNGHYTEPRVAHVPGIDSWPGKQIHSHNYRVPDQFKDQVVVVIGNFASGADISRDITGVAKEVHIASRSNPSKTYSKLPGSNNLWLHSMIESVHEDGTIVFQNGKVVQADTIVHCTGYKYHFPFLNTNGYITVEDNCVGPLYEHVFPPALAPGLSFIGLPWMTLQFFMFELQSKWVAAALSGRVTLPSEEKMMEDVTAYYAKREAFGQPKRYTHRLGGGQVDYLNWIAEQIGAPPGEQWRYQEINGGYYRLATQSDTFRDKWDDDHLIVEAYEDFLRQKLISSLPSQLLES

SEQ ID NO: 11 - DNA, 1101 bp, Arabidopsis thaliana
atgtcaagaaagccatgttgcgtcggagaaggcttgaagaaaggagcatggaccaccgaggaggacaagaaactcatctcttacatccacgaccacggcgagggaggctggcgcgacattccccaaaaagctgggttgaaacggtgtggaaagagttgtagactgcgatggaccaactaccttaaacctgagatcaaaagaggcgagtttagttcagaggaagagcagattatcatcatgcttcatgcttctcgtggcaacaagtggtcggtcatagcgagacatttacctagaagaacagacaacgagatcaagaactactggaacacgcatctcaaaaaacgtttgatggaacagggtattgatcccgtgactcacaagccactggcttctagttccaaccctacggtcgatgagaatttgaattccccaaatgcctctagttccgacaagcaatactcccgatcgagctcaatgccttttctgtctcgtcctcctccatccagttgcaacatggtttccaaggtctccgagcttagcagcaatgatgggacaccgattcaaggcagttccttgagttgcaagaaacgtttcaagaaatcaagttctacatcaaggctcttgaacaaagttgcggctaaggccacttccatcaaagatatattgtcggcttccatggaaggtagcttgagtgctactacaatatcacatgcaagcttttttaatggcttcactgagcagattcgcaatgaagaggatagttctaacacatccctgacaaatactcttgctgaatttgatcccttctccccatcatcgttgtaccccgaacatgagatcaatgctacttctgatctcaacatggaccaagattacgatttttcacaatttttcgaaaaattcggaggagataaccacaatgaggagaacagtatgaatgatctccttatgtccgatgtttcccaagaagtctcatcaactagcgttgatgatcaagacaatatggtaggaaacttcgagggatggtcaaattatcttcttgaccataccaattttatgtatgacaccgactcagactcgcttgaaaagcatttcatatga

SEQ ID NO: 12 - protein, 366 aa, Arabidopsis thaliana
MSRKPCCVGEGLKKGAWTTEEDKKLISYIHDHGEGGWRDIPQKAGLKRCGKSCRLRWTNYLKPEIKRGEFSSEEEQIIIMLHASRGNKWSVIARHLPRRTDNEIKNYWNTHLKKRLMEQGIDPVTHKPLASSSNPTVDENLNSPNASSSDKQYSRSSSMPFLSRPPPSSCNMVSKVSELSSNDGTPIQGSSLSCKKRFKKSSSTSRLLNKVAAKATSIKDILSASMEGSLSATTISHASFFNGFTEQIRNEEDSSNTSLTNTLAEFDPFSPSSLYPEHEINATSDLNMDQDYDFSQFFEKFGGDNHNEENSMNDLLMSDVSQEVSSTSVDDQDNMVGNFEGWSNYLLDHTNFMYDTDSDSLEKHFI

SEQ ID NO: 13 - DNA, 1011 bp, Arabidopsis thaliana
atgtcaagaaagccatgttgtgtgggagaaggactgaagaaaggagcatggactgccgaagaagacaagaaactcatctcttacattcatgaacacggtgaaggaggctggcgtgacattccccaaaaagctggactaaaacgatgtggaaagagttgtagattgcgatgggctaactatttgaaacctgacatcaagagaggagagtttagctatgaggaggaacagattatcatcatgctacacgcttctcgcggcaacaagtggtcagtcatagcgagacatttgcccaaaagaacagataacgagattaagaactactggaacacgcatctcaaaaagctcctgatcgataagggaatcgatcccgtgacccacaagccacttgcctatgactcaaacccggatgagcaatcgcaatcgggttccatctctccaaagtctcttcctccttcaagctccaaaaatgtaccggagataaccagcagtgacgagacaccgaaatatgatgcttccttgagctccaagaaacgttgttttaagagatcgagttctacatcaaaactgttaaacaaagttgcagctagggcttcttccatgggaactatactaggcgcctccatcgaaggaaccttgatcagctctacaccgttgtcttcatgtctaaatgatgacttttctgaaacaagtcaatttcagatggaagaatttgatccattctatcagtcatctgaacacataattgatcatatgaaagaagatatcagcatcaacaattccgaatacgatttctcgcagtttctcgagcagtttagtaacaacgaaggggaagaagctgacaatactggaggaggatataaccaagatcttcttatgtctgatgtctcatcaacaagcgttgatgaagacgagatgatgcaaaacataactggttggtcaaattatctccttgaccattccgatttcaattatgacacgagccaagattacgacgacaagaacttcatatga

SEQ ID NO: 14 - protein, 336 aa, Arabidopsis thaliana
MSRKPCCVGEGLKKGAWTAEEDKKLISYIHEHGEGGWRDIPQKAGLKRCGKSCRLRWANYLKPDIKRGEFSYEEEQIIIMLHASRGNKWSVIARHLPKRTDNEIKNYWNTHLKKLLIDKGIDPVTHKPLAYDSNPDEQSQSGSISPKSLPPSSSKNVPEITSSDETPKYDASLSSKKRCFKRSSSTSKLLNKVAARASSMGTILGASIEGTLISSTPLSSCLNDDFSETSQFQMEEFDPFYQSSEHIIDHMKEDISINNSEYDFSQFLEQFSNNEGEEADNTGGGYNQDLLMSDVSSTSVDEDEMMQNITGWSNYLLDHSDFNYDTSQDYDDKNFI

SEQ ID NO: 15 - DNA, 1017 bp, Arabidopsis thaliana
atgtcaaagagaccatattgtatcggagaaggactgaagaaaggagcatggactacagaagaggataaaaaactcatctcttatatccacgaccacggtgaaggaggctggcgtgacattccagaaaaagctgggctgaaacggtgtggaaagagttgtagattacggtggactaactatttgaaaccagatatcaagagaggagagtttagctatgaggaagagcagattatcatcatgcttcatgcatctcgtggcaataagtggtctgtcatagctagacatttgccaaaaagaacggataacgaggtcaaaaactattggaacacacatctcaagaaacgtttaatcgatgatggcattgatcccgtgacacacaagccactagcttcttctaaccctaatccagttgagcccatgaagttcgatttccaaaagaaatccaatcaggatgagcactcttcacagtctagttctacaactccagcatctcttcccctttcctcgaatttgaacagtgttaaatccaaaattagcagtggtgagacgcagatagaaagtggtcacgtgagctgcaagaaacgttttggacgatcgagctctacatcaaggttgttaaacaaagttgcagctagagcttcttccatcggcaacatcttatcaacatccatagaaggaaccttgagatctcctgcatcatcttcaggactcccagactcgttctctcaatcatatgagtacatgatcgataacaaagaagatctcggtacgagcattgatctcaacatccccgagtatgatttcccacagtttcttgagcaactcattaacgatgacgacgaaaatgagaacattgttgggcccgaacaagatctccttatgtccgatttcccatcaacattcgttgatgaagacgatatacttggagacataaccagttggtcaacttatcttcttgaccatcccaattttatgtatgaatcggatcaagattccgacgagaagaacttcttatga

SEQ ID NO: 16 - protein, 338 aa, Arabidopsis thaliana
MSKRPYCIGEGLKKGAWTTEEDKKLISYIHDHGEGGWRDIPEKAGLKRCGKSCRLRWTNYLKPDIKRGEFSYEEEQIIIMLHASRGNKWSVIARHLPKRTDNEVKNYWNTHLKKRLIDDGIDPVTHKPLASSNPNPVEPMKFDFQKKSNQDEHSSQSSSTTPASLPLSSNLNSVKSKISSGETQIESGHVSCKKRFGRSSSTSRLLNKVAARASSIGNILSTSIEGTLRSPASSSGLPDSFSQSYEYMIDNKEDLGTSIDLNIPEYDFPQFLEQLINDDDENENIVGPEQDLLMSDFPSTFVDEDDILGDITSWSTYLLDHPNFMYESDQDSDEKNFL

SEQ ID NO: 17 - DNA, 3885 bp, Artificial sequence
  Synthetic sequence Myb28 for 35Senh-construct
caatgtaaatgctcggaagtgagtcgttgcgaaaatttaggtttgtaaaatgaaggattatggtgagttttagtttgcaaaataactaaaatattatgggaccaaggaaataatcaagaataagtgaagatacactatgggaccgtttaagtaggttgacatatataactgactggaaccagcggatcttagggatataatcaatacttattgactaaaattttcccaaaagaaagaagaatcaaatgattactctatgtagtaacccaaactgatcctaacaaaattgtagaaatgcagatggtttaaatatgtggcgctctcataaaactcctacttcaggtaatctttttacacagtttggagctatcgtagctcttaacattttcactccagcaatgactagaaccaacagaacaatgagagattggcttctatccatagaaagcttcaacacgaaaaccgaccaaaacgaaatgttaaacccaagccttcttcaagcatagctgtatcatattctatcttccttgtaagagttccttttgttaaaaactaaatactaaatccgacttaaagaataataatcaagaacttcaaaatagcaaagtaaaatatacacacgcacaaattgataagagttcacttagcttgcagtacgagaactaggcaggggcagacctagcttaagagtgtaggtgtggcaggtgtttaattatatagaatttactttgtggcactaacatatttttgttttataatgcaaaataagatgttaaatttgattaaatttatatacaatacaagtttgtgttctatgtaaaatatttttctagatcaaacaaggtctagttttaaacgatccatgggagtataaattttatctttttcactctaccttgaaaaatgcgcatgagataaaatcataggtacatatacatacgtgaagaatagcatcagaaaatattgttctaactattccgataactaacaaaaccttaggaaacctcatcaagactagttcgaattaaatcataaggtttaggttgagagagtcaaagagggaatgatataaatagaagaatattttttgtttaagaatgatttttagacaatggaaagaagaatatgttaaggtggtatagacgacgagcaataatacaacagccacaaaagtggcaaacaaaaaggacctacgctggaaaaaaaacacgtgatgttacaatcaccctttcattctcaatgatgaacaataatgtttattattgataagaaaaacaataatgtaaatttatactttctcgttaaccagatttgttttttcattatgcgtttgcagtataaaaatagtaaaatacgttttaagtattaaactgtttgatagtttttttttatatatatacctaacagaaaccaactatttaaacaatacaaaaatatctgcaaagatatatatataatacaatcgaattcttaaaagttatatatatttgcaaacgtccctttagttattcccctccaactctccatgttggatcaatcattcaatttttttttaataaccaaaagttaaatgtacaaatatgcaagaacctacaggtacgtttacgtgatatataaattaaaatattgcatctcgtaccgaagcgcattaccgtatttaaaatacctgaaagtaggaaaatatagtactatacaaacaccacttttcggacattattttcatagaaaagttacgaattatcctttttaactattgatctatttaaataatttactaaccataactatcttgttacgttttcacaaaaaaaaaaaaaaaaaaatctcattacgtacgtgtatatatatggaatagctcataacctcaccactaccacagaaatcatgcctcttggttcttttccataagcttataacatatattttttttaaaatctactctgcgttaaaaaaatgaaaacacgtagcagcagtgtgggtaagatcaaagggtgtttctcgatcagtttcatattcagatgtatcagagttctcattaacagatctgtttctttttccttatctgattaaacaatttccttcagaattttacttttttgaacatatatagtttttctctgttcctatatcttgagttttgtgagaggttaattatatgaaattttacgcattattgttcatctatatcgaaaaacaatgtcaagaaagccatgttgcgtcggagaaggcttgaagaaaggagcatggaccaccgaggaggacaagaaactcatctcttacatccacgaccacggcgagggaggctggcgcgacattccccaaaaagctggtttatacaaatctatacatacactcatttttgtacttgttgtagaaaattgttctgataaacatattgtgtctgattagggttgaaacggtgtggaaagagttgtagactgcgatggaccaactaccttaaacctgagatcaaaagaggcgagtttagttcagaggaagagcagattatcatcatgcttcatgcttctcgtggcaacaagtacgtttctatgtttctatgtgtgtgcgtggaccctcgaatgtgaaatgaatttcatgaaaaagttttcatataatatttattatgtagacataatcatcattttaatcttggtctccgatctatcttattttctttaggtggtcggtcatagcgagacatttacctagaagaacagacaacgagatcaagaactactggaacacgcatctcaaaaaacgtttgatggaacagggtattgatcccgtgactcacaagccactggcttctagttccaaccctacggtcgatgagaatttgaattccccaaatgcctctagttccgacaagcaatactcccgatcgagctcaatgccttttctgtctcgtcctcctccatccagttgcaacatggtttccaaggtctccgagcttagcagcaatgatgggacaccgattcaaggcagttccttgagttgcaagaaacgtttcaagaaatcaagttctacatcaaggctcttgaacaaagttgcggctaaggccacttccatcaaagatatattgtcggcttccatggaaggtagcttgagtgctactacaatatcacatgcaagcttttttaatggcttcactgagcagattcgcaatgaagaggatagttctaacacatccctgacaaatactcttgctgaatttgatcccttctccccatcatcgttgtaccccgaacatgagatcaatgctacttctgatctcaacatggaccaagattacgatttttcacaatttttcgaaaaattcggaggagataaccacaatgaggagaacagtatgaatgatctccttatgtccgatgtttcccaagaagtctcatcaactagcgttgatgatcaagacaatatggtaggaaacttcgagggatggtcaaattatcttcttgaccataccaattttatgtatgacaccgactcagactcgcttgaaaagcatttcatatgagtcttcatatccaaacagaaaggtttcaaactattcgacgacttaaaataatggttctgtacccaaggttagtcgattactaactcgctcgaacgagatattgtgtatgtattaattagtatttgggttgtttactatatgtccaaggcgtgtttattacgatgttaaacaagggttaatcttaacacttaagtttccccaagaataaataaaatagggtttgagttagggtttctcttacattgagaaccatgcatgtaacctcgcgaatcaattggtaattgatttgtgcgggccacgatgtttatactaatatttctttctaaagcttgttttatttatcttatttcgtagtagtacttcccatt

SEQ ID NO: 18 - DNA, 3823 bp, Artificial sequence
  Synthetic sequence Myb29 for 35Senh-construct
attttcaacgattgcgttgtttcccaaattatgaattggaactttggtagcatcgcaatatatacgacgtttgggtttggcccatgctgccatgcatagcaatagttttaaatacatgtggttggtaatatagaaaatggtttgaaagaccacaaactttgatcagtgatcgattcggtagggccacaaataacaatgttttcgccacatggtcatactttacgttttcgatgagaaaatatttaaacagtttgttcgtcaaatttcgattaactagaacaaaattcataaacgagagagacagaataattcgagagagctagagtgagggtaactagaaagatagtaactgattttgtatctaataattaattcattaatttaaaatcaatgataaatcactttgatggttgtggccttgtgggtaattataattaacacgtaccattttttatcaaggcatttttaaacattttgtttgtttttattgaagttttcttctcactattcaaaaacgtaaaaccctaacaaaaaaaagtaaaagtagaactgtttacaagtctggctgaatgggttgattgactcgacaaaagattttccatgtggattaatagaacaaattttaataatatatacgaaattgatgtattttcttttctttcgatcactattaatgtcttaaataataaaaacatatagtacattttacagattataaatttatgttgtgttttattttgagttttggcttgaattttttttttttttttgttgttgttgttaaagtggtttgaatctgtattggttacaaataaaacaaataatgttacgttcattttgtctgtagatatttttcttacaacttatgcagctatctttggggtttcattttgagtgtggatgtttggttttgagttaactctgcatgttccgaagaggcgtaactaaaataaaagaaactacttgagatgcgagatgtgaaatgtgattagatgagaaaaaacgaacattaataattgagcaaaataactttatttaaattttgaattcagcgttagtgttacacccaaagtggcaaacaaataggaccgatgttgaaaaagaacacacgtgatataaaatgtactgagagaaaattatttgcattagatgacaataaatacaataataatgaatagatgaaataacttttagttgacgaaaaaaaaaaaaaacttttaatctattttattcactagatcaaaaagcatgtttcagacagttttattcttatcattcaattatttcacaacgtataattttagtttattttcgtaatttgttaatatacgtatcaattgaatatttttgacggtttttattatgtcatttaattatttaagggaacatagtttattttaaaatgcagttctattttacaaaaaaaaagaaaaaaaaaattgcagttctacgttgacatctagctgatcaactattcatcatatatacttgtataatctattattttaagttctatattattattaatatgttaaatatagatatatctatttaagaaaatatcatataaatatatgttataaatctatatatagaaaaaataaagcacagaattttgtcccacattctgtcgatacgtactcgagcttatgaagttgttcttttctaattatattttttcccattgccctttatcaaatcaactctaataaaaatatatggtaacttatgaagttgtcatgtatttatgatatttctctttgggtcggcactgtatttgtgatgttgattatttatctagtggcagaaaatattccataagtctctctcaaaccatttgaatagttccaaaaacatcttgtcactaacactcactcttgatgagttttttttttttttttttggggggtcaaagtactcttgatgatgagttgatattcttatttaaaaaaagcttattacttatttaagttatttcaaaaagtacattctacacgagtgccaggcttatatatatgcataaacatatataattatgcatggaggagtagtagcttgcaatgtcttgaaactttgatatatcttctcctagtctttcttttaaatgtttaatatgaaaacacaaaatcctacaacggtcgtctaccacagtttctcagtcagtttcatattcagatgcatcagagttctcatcaagagatctatcagtctattgccttaaactcgacgacattctgttttttttttcttttcttattttttctttttcttatttcttacctataggttgtatgtaaatctatatcaaaaaaagaagaaaaacaagatgtcaagaaagccatgttgtgtgggagaaggactgaagaaaggagcatggactgccgaagaagacaagaaactcatctcttacattcatgaacacggtgaaggaggctggcgtgacattccccaaaaagctggtatatatgtgctttattattatgtatatattttaaaacactttttacatatatataactataattgttgtttttatgacaaatgatggtgtttaggactaaaacgatgtggaaagagttgtagattgcgatgggctaactatttgaaacctgacatcaagagaggagagtttagctatgaggaggaacagattatcatcatgctacacgcttctcgcggcaacaagtaaaatcctagcttgccgaaatccatataaataagggtatatataattaacacattattaaagtttatatatatgttttacttaaaaggtggtcagtcatagcgagacatttgcccaaaagaacagataacgagattaagaactactggaacacgcatctcaaaaagctcctgatcgataagggaatcgatcccgtgacccacaagccacttgcctatgactcaaacccggatgagcaatcgcaatcgggttccatctctccaaagtctcttcctccttcaagctccaaaaatgtaccggagataaccagcagtgacgagacaccgaaatatgatgcttccttgagctccaagaaacgttgttttaagagatcgagttctacatcaaaactgttaaacaaagttgcagctagggcttcttccatgggaactatactaggcgcctccatcgaaggaaccttgatcagctctacaccgttgtcttcatgtctaaatgatgacttttctgaaacaagtcaatttcagatggaagaatttgatccattctatcagtcatctgaacacataattgatcatatgaaagaagatatcagcatcaacaattccgaatacgatttctcgcagtttctcgagcagtttagtaacaacgaaggggaagaagctgacaatactggaggaggatataaccaagatcttcttatgtctgatgtctcatcaacaagcgttgatgaagacgagatgatgcaaaacataactggttggtcaaattatctccttgaccattccgatttcaattatgacacgagccaagattacgacgacaagaacttcatatgatccgttgattgcttaccggactagagttgaccggttaatgtcatatggttctcttagatatttgtcaagttatagtaaaggtccactatagggtcactatatattaatattcagtaatggattctcttagttagagaaccttgtgatgccgtggatcaattagtatttgatttgcgggagacacgagttttttttccttctattgttgtttgtggatttacgtactataaataataaataaaacacccatttgattgcaagcgttcactgtactaaaaccatttgatttaaagtttgagcc

SEQ ID NO: 19 - DNA, 3072 bp, Artificial sequence
  Synthetic sequence Myb76, for 35Senh-construct
aagcgttcactgtactaaaaccatttgatttaaagtttgagccttagtttgtctgacagtctgagccatgttaccaaaaacaatgaaaaatatgtaacacattttaggtttttggtgatatgaaactccgaagaaacaaatccctactgactactgagaaagtcgataagcttttttgtggataagtttcatggatatattagaagtagtaaccattaaccaacaaaaaaatagcttaagtgagttatcaagggatcgatgaacaattatgagatccaatgtgtttttgttaagaggcaaaatccgatgcagtctctatgagacaaaatttccatgggaaaaacagagagttctgaagtctctctaccttaaacatgtgcaagccttagcttcaaatgctccgtaaggttttcatttaaaaacatgaaataagatagagaaatgatacttgatccaactgatgaagattaacaagataattttgaagcaacttctgtttgtataatatgtcgtacaaaatctgctaccaatttagaggccaaattattttcttttctagacagtttgtgaggtgggcagctgaaggtgtttaagccaagattctcaagatttataaatcttgaatcgaattaagctatcagccggaaattaggaaatgatatgcatatagggactaaagatatagttgttgcattaaaaagcttaaagagagaagtggatgtgaaaagaaaaaaaccacagatttttgcacacaatcttgtgtgttgattgatatccaagtagactaattagactgctttgttctacacgataattggttgtttttagatatcaatacgaaacatgttaaaatgtgaaaatattttagattagatgataacacctgaatttaatgacaaaaaaaaaaaaaagtggatagagactagagggacagcaaggctgtgtgacatatatgggcagatagacaaagaagccgaaaaacgtgcaccgtccaagattctggctactatacctaatttccttcccgcagggacttgacaaatatcactatctgccatttttagttttattttgtattggtgtcaaagaattgaaataatgaacaacggtcgtaaaaagatgtaaatggtgtttgattgattaatgtttttttttttttcttagtatattttagcaattgcatattatcatataacattaattaataattattgtgttgtagataaatgtcatgcataatgcagtaataaatgtgtgtgcatatattatatatacacgttaattagatgctaaaatgagtgacatatcttttaattctttgataacaccatttccataaatcattgtaaaacttaccttataacaaaaaattaataaatgttataggggtcaattgacccccataactctacactagccccacctctgcggataagcttaacatgtctattaatattcattagtttacgtggtttaaaagtttattgtcacgagtgcatgacacttaccgtgatgttgactatatgaagaggtagatcgtacgtgtacaaatgacttcatagatctttgatcttttttttcttcttcttcttttttggtaatattctttagttttatttgatctattgtcgttgtaatgatctttgattacaaaggaaaaaaaaaactaagacccttgacgaaaataataaccgtattcgtaatctctgatatcctacattatgtactatttctgatttttgtttcttatacgcacttttgttctagatataactaagaaaatgtcaaagagaccatattgtatcggagaaggactgaagaaaggagcatggactacagaagaggataaaaaactcatctcttatatccacgaccacggtgaaggaggctggcgtgacattccagaaaaagctggtacataactatatatagacgcatttgtgtctctataatatgaatttattcacaatctgttactaatatgtattaattattctcttaattgatcatttgatctttatctgctttttttcgagtttagggctgaaacggtgtggaaagagttgtagattacggtggactaactatttgaaaccagatatcaagagaggagagtttagctatgaggaagagcagattatcatcatgcttcatgcatctcgtggcaataagtacgtatggcatttctctaggcttgtttgtgctcatatcagtttagtgaagacatgatcatcaatgttttgatatatatgtaccctgtgtttttattttattttactaggtggtctgtcatagctagacatttgccaaaaagaacggataacgaggtcaaaaactattggaacacacatctcaagaaacgtttaatcgatgatggcattgatcccgtgacacacaagccactagcttcttctaaccctaatccagttgagcccatgaagttcgatttccaaaagaaatccaatcaggatgagcactcttcacagtctagttctacaactccagcatctcttcccctttcctcgaatttgaacagtgttaaatccaaaattagcagtggtgagacgcagatagaaagtggtcacgtgagctgcaagaaacgttttggacgatcgagctctacatcaaggttgttaaacaaagttgcagctagagcttcttccatcggcaacatcttatcaacatccatagaaggaaccttgagatctcctgcatcatcttcaggactcccagactcgttctctcaatcatatgagtacatgatcgataacaaagaagatctcggtacgagcattgatctcaacatccccgagtatgatttcccacagtttcttgagcaactcattaacgatgacgacgaaaatgagaacattgttgggcccgaacaagatctccttatgtccgatttcccatcaacattcgttgatgaagacgatatacttggagacataaccagttggtcaacttatcttcttgaccatcccaattttatgtatgaatcggatcaagattccgacgagaagaacttcttatgatctgtctatagatggcttgtcaatttcccaatgttga

SEQ ID NO: 20 - DNA, 340 bp, Artificial sequence
  Synthetic sequence 35Senhancer sequence
cttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctcc

SEQ ID NO: 21 - DNA, 33 bp, Artificial sequence
  Synthetic sequence Primer number 20; BamHI-p35Senh-F
aataacaggatcccttcgtcaacatggtggagc

SEQ ID NO: 22 - DNA, 43 bp, Artificial sequence
  Synthetic sequence Primer number 23; Myb76-p35Senh-R
ttttagtacagtgaacgcttggagatatcacatcaatccactt

SEQ ID NO: 23 - DNA, 42 bp, Artificial sequence
  Synthetic sequence Primer number 24; p35S-enh-myb28-F
ttgattgatgtgatatctcctttgcaaaatgatagtggagaa

SEQ ID NO: 24 - DNA, 43 bp, Artificial sequence
  Synthetic sequence Primer number 26; p35S-enh-myb76-F
ttgattgatgtgatatctccaagcgttcactgtactaaaacca

SEQ ID NO: 25 - DNA, 36 bp, Artificial sequence
  Synthetic sequence Primer number 27; PstI-myb28-R
aataacactgcagttgatgactattatgggcactga

SEQ ID NO: 26 - DNA, 35 bp, Artificial sequence
  Synthetic sequence Primer number 29; PstI-myb76-R
aataacactgcagtcaacattgggaaattgacaag

SEQ ID NO: 27 - DNA, 36 bp, Artificial sequence
  Synthetic sequence Primer number 41; PstI-myb29-R
aataacactgcaggtagggatttgtttcttcggagt

SEQ ID NO: 28 - DNA, 22 bp, Artificial sequence
  Synthetic sequence Primer number 49; pCambia2300-F
gagcggataacaatttcacaca

SEQ ID NO: 29 - DNA, 33 bp, Artificial sequence
  Synthetic sequence Primer number 54; SacI-p35Senh-F
aataacagagctccttcgtcaacatggtggagc

SEQ ID NO: 30 - DNA, 36 bp, Artificial sequence
  Synthetic sequence Primer number 55; SacI-p35Senh-R
aataacagagctcggagatatcacatcaatccactt

SEQ ID NO: 31 - DNA, 32 bp, Artificial sequence
  Synthetic sequence Primer number 59; USER-Myb29-R
ggtttaauggctcaaactttaaatcaaatggt

SEQ ID NO: 32 - DNA, 30 bp, Artificial sequence
  Synthetic sequence Primer number 60; USER-nMyb28-F
ggcttaaucaatgtaaatgctcggaagtga

SEQ ID NO: 33 - DNA, 35 bp, Artificial sequence
  Synthetic sequence Primer number 61; USER-nMyb28-R
ggtttaauaatgggaagtactactacgaaataaga

SEQ ID NO: 34 - DNA, 33 bp, Artificial sequence
  Synthetic sequence Primer number 66; EcoRI-35Senh-F
aataacagaattccttcgtcaacatggtggagc

SEQ ID NO: 35 - DNA, 25 bp, Artificial sequence
  Synthetic sequence Primer number 67; outsMyb29-F
ccttggttacaatatatgcagcttt

SEQ ID NO: 36 - DNA, 30 bp, Artificial sequence
  Synthetic sequence Primer number 68; USER-snMyb29-F short
ggcttaauattttcaacgattgcgttgttt

SEQ ID NO: 37 - DNA, 32 bp, Artificial sequence
  Synthetic sequence Primer number 75; MYB76 f U
ggcttaauatgtcaaagagaccatattgtatc

SEQ ID NO: 38 - DNA, 32 bp, Artificial sequence
  Synthetic sequence Primer number 76; MYB76 r U
ggtttaautcataagaagttcttctcgtcgga

SEQ ID NO: 39 - DNA, 24 bp, Artificial sequence
  Synthetic sequence Primer number 45; MYB76 f
atgtcaaagagaccatattgtatc

SEQ ID NO: 40 - DNA, 24 bp, Artificial sequence
  Synthetic sequence Primer number 46; MYB76 r
tcataagaagttcttctcgtcgga

SEQ ID NO: 41 - DNA, 24 bp, Artificial sequence
  Synthetic sequence Primer number 156; MYB28 f
atgtcaagaaagccatgttgcgtc

SEQ ID NO: 42 - DNA, 24 bp, Artificial sequence
  Synthetic sequence Primer number 50; MYB28 r
tcatatgaaatgcttttcaagcga

SEQ ID NO: 43 - DNA, 31 bp, Artificial sequence
  Synthetic sequence Primer number 157; MYB28 f U
ggcttaauatgtcaagaaagccatgttgcgt

SEQ ID NO: 44 - DNA, 29 bp, Artificial sequence
  Synthetic sequence Primer number 74; MYB28 r U
ggtttaautcatatgaaatgcttttcaag

SEQ ID NO: 45 - DNA, 20 bp, Artificial sequence
  Synthetic sequence Primer number 100; MYB29 f
atgtcaagaaagccatgttg

SEQ ID NO: 46 - DNA, 21 bp, Artificial sequence
  Synthetic sequence Primer number 101; MYB29 r
tcatatgaagttcttgtcgtc

SEQ ID NO: 47 - DNA, 29 bp, Artificial sequence
  Synthetic sequence Primer number 71; MYB29 r U
ggtttaautcatatgaagttcttgtcgtc

SEQ ID NO: 48 - DNA, 28 bp, Artificial sequence
  Synthetic sequence Primer number 72; MYB29 f U
ggcttaauatgtcaagaaagccatgttg

SEQ ID NO: 49 - DNA, 24 bp, Artificial sequence
  Synthetic sequence Primer number 104; At1g62560 f
atggcaccagctcaaaaccaaatc

SEQ ID NO: 50 - DNA, 26 bp, Artificial sequence
  Synthetic sequence Primer number 105; At1g62560 r
tcatcttccattttcgaggtaataag

SEQ ID NO: 51 - DNA, 32 bp, Artificial sequence
  Synthetic sequence Primer number 102; At1g62560 f U
ggcttaauatggcaccagctcaaaaccaaatc

SEQ ID NO: 52 - DNA, 34 bp, Artificial sequence
  Synthetic sequence Primer number 103; At1g62560 r U
ggtttaautcatcttccattttcgaggtaataag

SEQ ID NO: 53 - DNA, 24 bp, Artificial sequence
  Synthetic sequence Primer number 109; At1g65860 f
atggcaccaactcaaaacacaatc

SEQ ID NO: 54 - DNA, 23 bp, Artificial sequence
  Synthetic sequence Primer number 110; At1g65860 r
tcatgattcgaggaaataagaag

SEQ ID NO: 55 - DNA, 22 bp, Artificial sequence
  Synthetic sequence Primer number 111; At1g65860 r - stop
tgattcgaggaaataagaagga

SEQ ID NO: 56 - DNA, 32 bp, Artificial sequence
  Synthetic sequence Primer number 107; At1g65860 f U
ggcttaauatggcaccaactcaaaacacaatc

SEQ ID NO: 57 - DNA, 31 bp, Artificial sequence
  Synthetic sequence Primer number 108; At1g65860 r U
ggtttaautcatgattcgaggaaataagaag